SEQUENCE LISTING (28 sequences)

SEQ ID NO: 1 - protein, 470 aa, Homo sapiens
MHSFPPLLLLLFWGVVSHSFPATLETQEQDVDLVQKYLEKYYNLKNDGRQVEKRRNSGPVVEKLKQMQEFFGLKVTGKPDAETLKVMKQPRCGVPDVAQFVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKNL

SEQ ID NO: 2 - DNA, 1410 bp, Homo sapiens
atgcacagctttcctccactgctgctgctgctgttctggggtgtggtgtctcacagcttcccagcgactctagaaacacaagagcaagatgtggacttagtccagaaatacctggaaaaatactacaacctgaagaatgatgggaggcaagttgaaaagcggagaaatagtggcccagtggttgaaaaattgaagcaaatgcaggaattctttgggctgaaagtgactgggaaaccagatgctgaaaccctgaaggtgatgaagcagcccagatgtggagtgcctgatgtggctcagtttgtcctcactgaggggaaccctcgctgggagcaaacacatctgacctacaggattgaaaattacacgccagatttgccaagagcagatgtggaccatgccattgagaaagccttccaactctggagtaatgtcacacctctgacattcaccaaggtctctgagggtcaagcagacatcatgatatcttttgtcaggggagatcatcgggacaactctccttttgatggacctggaggaaatcttgctcatgcttttcaaccaggcccaggtattggaggggatgctcattttgatgaagatgaaaggtggaccaacaatttcagagagtacaacttacatcgtgttgcggctcatgaactcggccattctcttggactctcccattctactgatatcggggctttgatgtaccctagctacaccttcagtggtgatgttcagctagctcaggatgacattgatggcatccaagccatatatggacgttcccaaaatcctgtccagcccatcggcccacaaaccccaaaagcgtgtgacagtaagctaacctttgatgctataactacgattcggggagaagtgatgttctttaaagacagattctacatgcgcacaaatcccttctacccggaagttgagctcaatttcatttctgttttctggccacaactgccaaatgggcttgaagctgcttacgaatttgccgacagagatgaagtccggtttttcaaagggaataagtactgggctgttcagggacagaatgtgctacacggataccccaaggacatctacagctcctttggcttccctagaactgtgaagcatatcgatgctgctctttctgaggaaaacactggaaaaacctacttctttgttgctaacaaatactggaggtatgatgaatataaacgatctatggatccaggttatcccaaaatgatagcacatgactttcctggaattggccacaaagttgatgcagttttcatgaaagatggatttttctatttctttcatggaacaagacaatacaaatttgatcctaaaacgaagagaattttgactctccagaaagctaatagctggttcaactgcaggaaaaatttg

SEQ ID NO: 3 - protein, 660 aa, Homo sapiens
MEALMARGALTGPLRALCLLGCLLSHAAAAPSPIIKFPGDVAPKTDKELAVQYLNTFYGCPKESCNLFVLKDTLKKMQKFFGLPQTGDLDQNTIETMRKPRCGNPDVANYNFFPRKPKWDKNQITYRIIGYTPDLDPETVDDAFARAFQVWSDVTPLRFSRIHDGEADIMINFGRWEHGDGYPFDGKDGLLAHAFAPGTGVGGDSHFDDDELWTLGEGQVVRVKYGNADGEYCKFPFLFNGKEYNSCTDTGRSDGFLWCSTTYNFEKDGKYGFCPHEALFTMGGNAEGQPCKFPFRFQGTSYDSCTTEGRTDGYRWCGTTEDYDRDKKYGFCPETAMSTVGGNSEGAPCVFPFTFLGNKYESCTSAGRSDGKMWCATTANYDDDRKWGFCPDQGYSLFLVAAHEFGHAMGLEHSQDPGALMAPIYTYTKNFRLSQDDIKGIQELYGASPDIDLGTGPTPTLGPVTPEICKQDIVFDGIAQIRGEIFFFKDRFIWRTVTPRDKPMGPLLVATFWPELPEKIDAVYEAPQEEKAVFFAGNEYWIYSASTLERGYPKPLTSLGLPPDVQRVDAAFNWSKNKKTYIFAGDKFWRYNEVKKKMDPGFPKLIADAWNAIPDNLDAVVDLQGGGHSYFFKGAYYLKLENQSLKSVKFGSIKSDWLGC

SEQ ID NO: 4 - DNA, 3069 bp, Homo sapiens
tgtttccgctgcatccagacttcctcaggcggtggctggaggctgcgcatctggggctttaaacatacaaagggattgccaggacctgcggcggcggcggcggcggcgggggctggggcgcgggggccggaccatgagccgctgagccgggcaaaccccaggccaccgagccagcggaccctcggagcgcagccctgcgccgcggaccaggctccaaccaggcggcgaggcggccacacgcaccgagccagcgacccccgggcgacgcgcggggccagggagcgctacgatggaggcgctaatggcccggggcgcgctcacgggtcccctgagggcgctctgtctcctgggctgcctgctgagccacgccgccgccgcgccgtcgcccatcatcaagttccccggcgatgtcgcccccaaaacggacaaagagttggcagtgcaatacctgaacaccttctatggctgccccaaggagagctgcaacctgtttgtgctgaaggacacactaaagaagatgcagaagttctttggactgccccagacaggtgatcttgaccagaataccatcgagaccatgcggaagccacgctgcggcaacccagatgtggccaactacaacttcttccctcgcaagcccaagtgggacaagaaccagatcacatacaggatcattggctacacacctgatctggacccagagacagtggatgatgcctttgctcgtgccttccaagtctggagcgatgtgaccccactgcggttttctcgaatccatgatggagaggcagacatcatgatcaactttggccgctgggagcatggcgatggatacccctttgacggtaaggacggactcctggctcatgccttcgccccaggcactggtgttgggggagactcccattttgatgacgatgagctatggaccttgggagaaggccaagtggtccgtgtgaagtatggcaacgccgatggggagtactgcaagttccccttcttgttcaatggcaaggagtacaacagctgcactgatactggccgcagcgatggcttcctctggtgctccaccacctacaactttgagaaggatggcaagtacggcttctgtccccatgaagccctgttcaccatgggcggcaacgctgaaggacagccctgcaagtttccattccgcttccagggcacatcctatgacagctgcaccactgagggccgcacggatggctaccgctggtgcggcaccactgaggactacgaccgcgacaagaagtatggcttctgccctgagaccgccatgtccactgttggtgggaactcagaaggtgccccctgtgtcttccccttcactttcctgggcaacaaatatgagagctgcaccagcgccggccgcagtgacggaaagatgtggtgtgcgaccacagccaactacgatgacgaccgcaagtggggcttctgccctgaccaagggtacagcctgttcctcgtggcagcccacgagtttggccacgccatggggctggagcactcccaagaccctggggccctgatggcacccatttacacctacaccaagaacttccgtctgtcccaggatgacatcaagggcattcaggagctctatggggcctctcctgacattgaccttggcaccggccccacccccacactgggccctgtcactcctgagatctgcaaacaggacattgtatttgatggcatcgctcagatccgtggtgagatcttcttcttcaaggaccggttcatttggcggactgtgacgccacgtgacaagcccatggggcccctgctggtggccacattctggcctgagctcccggaaaagattgatgcggtatacgaggccccacaggaggagaaggctgtgttctttgcagggaatgaatactggatctactcagccagcaccctggagcgagggtaccccaagccactgaccagcctgggactgccccctgatgtccagcgagtggatgccgcctttaactggagcaaaaacaagaagacatacatctttgctggagacaaattctggagatacaatgaggtgaagaagaaaatggatcctggctttcccaagctcatcgcagatgcctggaatgccatccccgataacctggatgccgtcgtggacctgcagggcggcggtcacagctacttcttcaagggtgcctattacctgaagctggagaaccaaagtctgaagagcgtgaagtttggaagcatcaaatccgactggctaggctgctgagctggccctggctcccacaggcccttcctctccactgccttcgatacaccgggcctggagaactagagaaggacccggaggggcctggcagccgtgccttcagctctacagctaatcagcattctcactcctacctggtaatttaagattccagagagtggctcctcccggtgcccaagaatagatgctgactgtactcctcccaggcgccccttccccctccaatcccaccaaccctcagagccacccctaaagagatcctttgatattttcaacgcagccctgctttgggctgccctggtgctgccacacttcaggctcttctcctttcacaaccttctgtggctcacagaacccttggagccaatggagactgtctcaagagggcactggtggcccgacagcctggcacagggcagtgggacagggcatggccaggtggccactccagacccctggcttttcactgctggctgccttagaacctttcttacattagcagtttgctttgtatgcactttgtttttttctttgggtcttgttttttttttccacttagaaattgcatttcctgacagaaggactcaggttgtctgaagtcactgcacagtgcatctcagcccacatagtgatggttcccctgttcactctacttagcatgtccctaccgagtctcttctccactggatggaggaaaaccaagccgtggcttcccgctcagccctccctgcccctcccttcaaccattccccatgggaaatgtcaacaagtatgaataaagacacctactgagtggc

SEQ ID NO: 5 - protein, 477 aa, Homo sapiens
MKSLPILLLLCVAVCSAYPLDGAARGEDTSMNLVQKYLENYYDLKKDVKQFVRRKDSGPVVKKIREMQKFLGLEVTGKLDSDTLEVMRKPRCGVPDVGHFRTFPGIPKWRKTHLTYRIVNYTPDLPKDAVDSAVEKALKVWEEVTPLTFSRLYEGEADIMISFAVREHGDFYPFDGPGNVLAHAYAPGPGINGDAHFDDDEQWTKDTTGTNLFLVAAHEIGHSLGLFHSANTEALMYPLYHSLTDLTRFRLSQDDINGIQSLYGPPPDSPETPLVPTEPVPPEPGTPANCDPALSFDAVSTLRGEILIFKDRHFWRKSLRKLEPELHLISSFWPSLPSGVDAAYEVTSKDLVFIFKGNQFWAIRGNEVRAGYPRGIHTLGFPPTVRKIDAAISDKEKNKTYFFVEDKYWRFDEKRNSMEPGFPKQIAEDFPGIDSKIDAVFEEFGFFYFFTGSSQLEFDPNAKKVTHTLKSNSWLNC

SEQ ID NO: 6 - DNA, 1821 bp, Homo sapiens
acaaggaggcaggcaagacagcaaggcatagagacaacatagagctaagtaaagccagtggaaatgaagagtcttccaatcctactgttgctgtgcgtggcagtttgctcagcctatccattggatggagctgcaaggggtgaggacaccagcatgaaccttgttcagaaatatctagaaaactactacgacctcaaaaaagatgtgaaacagtttgttaggagaaaggacagtggtcctgttgttaaaaaaatccgagaaatgcagaagttccttggattggaggtgacggggaagctggactccgacactctggaggtgatgcgcaagcccaggtgtggagttcctgatgttggtcacttcagaacctttcctggcatcccgaagtggaggaaaacccaccttacatacaggattgtgaattatacaccagatttgccaaaagatgctgttgattctgctgttgagaaagctctgaaagtctgggaagaggtgactccactcacattctccaggctgtatgaaggagaggctgatataatgatctcttttgcagttagagaacatggagacttttacccttttgatggacctggaaatgttttggcccatgcctatgcccctgggccagggattaatggagatgcccactttgatgatgatgaacaatggacaaaggatacaacagggaccaatttatttctcgttgctgctcatgaaattggccactccctgggtctctttcactcagccaacactgaagctttgatgtacccactctatcactcactcacagacctgactcggttccgcctgtctcaagatgatataaatggcattcagtccctctatggacctccccctgactcccctgagacccccctggtacccacggaacctgtccctccagaacctgggacgccagccaactgtgatcctgctttgtcctttgatgctgtcagcactctgaggggagaaatcctgatctttaaagacaggcacttttggcgcaaatccctcaggaagcttgaacctgaattgcatttgatctcttcattttggccatctcttccttcaggcgtggatgccgcatatgaagttactagcaaggacctcgttttcatttttaaaggaaatcaattctgggccatcagaggaaatgaggtacgagctggatacccaagaggcatccacaccctaggtttccctccaaccgtgaggaaaatcgatgcagccatttctgataaggaaaagaacaaaacatatttctttgtagaggacaaatactggagatttgatgagaagagaaattccatggagccaggctttcccaagcaaatagctgaagactttccagggattgactcaaagattgatgctgtttttgaagaatttgggttcttttatttctttactggatcttcacagttggagtttgacccaaatgcaaagaaagtgacacacactttgaagagtaacagctggcttaattgttgaaagagatatgtagaaggcacaatatgggcactttaaatgaagctaataattcttcacctaagtctctgtgaattgaaatgttcgttttctcctgcctgtgctgtgactcgagtcacactcaagggaacttgagcgtgaatctgtatcttgccggtcatttttatgttattacagggcattcaaatgggctgctgcttagcttgcaccttgtcacatagagtgatctttcccaagagaaggggaagcactcgtgtgcaacagacaagtgactgtatctgtgtagactatttgcttatttaataaagacgatttgtcagttgtttt

SEQ ID NO: 7 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 8 - DNA, 2334 bp, Homo sapiens
agacacctctgccctcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccgggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagccgctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactagggctcccgtcctgctttgcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaaccttt

SEQ ID NO: 9 - protein, 582 aa, Homo sapiens
MSPAPRPPRCLLLPLLTLGTALASLGSAQSSSFSPEAWLQQYGYLPPGDLRTHTQRSPQSLSAAIAAMQKFYGLQVTGKADADTMKAMRRPRCGVPDKFGAEIKANVRRKRYAIQGLKWQHNEITFCIQNYTPKVGEYATYEAIRKAFRVWESATPLRFREVPYAYIREGHEKQADIMIFFAEGFHGDSTPFDGEGGFLAHAYFPGPNIGGDTHFDSAEPWTVRNEDLNGNDIFLVAVHELGHALGLEHSSDPSAIMAPFYQWMDTENFVLPDDDRRGIQQLYGGESGFPTKMPPQPRTTSRPSVPDKPKNPTYGPNICDGNFDTVAMLRGEMFVFKERWFWRVRNNQVMDGYPMPIGQFWRGLPASINTAYERKDGKFVFFKGDKHWVFDEASLEPGYPKHIKELGRGLPTDKIDAALFWMPNGKTYFFRGNKYYRFNEELRAVDSEYPKNIKVWEGIPESPRGSFMGSDEVFTYFYKGNKYWKFNNQKLKVEPGYPKSALRDWMGCPSGGRPDEGTEEETEVIIIEVDEEGGGAVSAAAVVLPVLLLLLVLAVGLAVFFFRRHGTPRRLLYCQRSLLDKV

SEQ ID NO: 10 - DNA, 3558 bp, Homo sapiens
cagaccccagttcgccgactaagcagaagaaagatcaaaaaccggaaaagaggagaagagcaaacaggcactttgaggaacaatcccctttaactccaagccgacagcggtctaggaattcaagttcagtgcctaccgaagacaaaggcgccccgagggagtggcggtgcgaccccagggcgtgggcccggccgcggagcccacactgcccggctgacccggtggtctcggaccatgtctcccgccccaagacccccccgttgtctcctgctccccctgctcacgctcggcaccgcgctcgcctccctcggctcggcccaaagcagcagcttcagccccgaagcctggctacagcaatatggctacctgcctcccggggacctacgtacccacacacagcgctcaccccagtcactctcagcggccatcgctgccatgcagaagttttacggcttgcaagtaacaggcaaagctgatgcagacaccatgaaggccatgaggcgcccccgatgtggtgttccagacaagtttggggctgagatcaaggccaatgttcgaaggaagcgctacgccatccagggtctcaaatggcaacataatgaaatcactttctgcatccagaattacacccccaaggtgggcgagtatgccacatacgaggccattcgcaaggcgttccgcgtgtgggagagtgccacaccactgcgcttccgcgaggtgccctatgcctacatccgtgagggccatgagaagcaggccgacatcatgatcttctttgccgagggcttccatggcgacagcacgcccttcgatggtgagggcggcttcctggcccatgcctacttcccaggccccaacattggaggagacacccactttgactctgccgagccttggactgtcaggaatgaggatctgaatggaaatgacatcttcctggtggctgtgcacgagctgggccatgccctggggctcgagcattccagtgacccctcggccatcatggcacccttttaccagtggatggacacggagaattttgtgctgcccgatgatgaccgccggggcatccagcaactttatgggggtgagtcagggttccccaccaagatgccccctcaacccaggactacctcccggccttctgttcctgataaacccaaaaaccccacctatgggcccaacatctgtgacgggaactttgacaccgtggccatgctccgaggggagatgtttgtcttcaaggagcgctggttctggcgggtgaggaataaccaagtgatggatggatacccaatgcccattggccagttctggcggggcctgcctgcgtccatcaacactgcctacgagaggaaggatggcaaattcgtcttcttcaaaggagacaagcattgggtgtttgatgaggcgtccctggaacctggctaccccaagcacattaaggagctgggccgagggctgcctaccgacaagattgatgctgctctcttctggatgcccaatggaaagacctacttcttccgtggaaacaagtactaccgtttcaacgaagagctcagggcagtggatagcgagtaccccaagaacatcaaagtctgggaagggatccctgagtctcccagagggtcattcatgggcagcgatgaagtcttcacttacttctacaaggggaacaaatactggaaattcaacaaccagaagctgaaggtagaaccgggctaccccaagtcagccctgagggactggatgggctgcccatcgggaggccggccggatgaggggactgaggaggagacggaggtgatcatcattgaggtggacgaggagggcggcggggcggtgagcgcggctgccgtggtgctgcccgtgctgctgctgctcctggtgctggcggtgggccttgcagtcttcttcttcagacgccatgggacccccaggcgactgctctactgccagcgttccctgctggacaaggtctgacgcccaccgccggcccgcccactcctaccacaaggactttgcctctgaaggccagtggcagcaggtggtggtgggtgggctgctcccatcgtcccgagccccctccccgcagcctccttgcttctctctgtcccctggctggcctccttcaccctgaccgcctccctccctcctgccccggcattgcatcttccctagataggtcccctgagggctgagtgggagggcggccctttccagcctctgcccctcaggggaaccctgtagctttgtgtctgtccagccccatctgaatgtgttgggggctctgcacttgaaggcaggaccctcagacctcgctggtaaaggtcaaatggggtcatctgctccttttccatcccctgacataccttaacctctgaactctgacctcaggaggctctgggcactccagccctgaaagccccaggtgtacccaattggcagcctctcactactctttctggctaaaaggaatctaatcttgttgagggtagagaccctgagacagtgtgagggggtggggactgccaagccaccctaagaccttgggaggaaaactcagagagggtcttcgttgctcagtcagtcaagttcctcggagatctgcctctgcctcacctaccccagggaacttccaaggaaggagcctgagccactggggactaagtgggcagaagaaacccttggcagccctgtgcctctcgaatgttagccttggatggggctttcacagttagaagagctgaaaccaggggtgcagctgtcaggtagggtggggccggtgggagaggcccgggtcagagccctgggggtgagcctgaaggccacagagaaagaaccttgcccaaactcaggcagctggggctgaggcccaaaggcagaacagccagagggggcaggaggggaccaaaaaggaaaatgaggacgtgcagcagcattggaaggctggggccgggcaggccaggccaagccaagcagggggccacagggtgggctgtggagctctcaggaagggccctgaggaaggcacacttgctcctgttggtccctgtccttgctgcccaggcagcgtggaggggaagggtagggcagccagagaaaggagcagagaaggcacacaaacgaggaatgaggggcttcacgagaggccacagggcctggctggccacgctgtcccggcctgctcaccatctcagtgaggggcaggagctggggctcgcttaggctgggtccacgcttccctggtgccagcacccctcaagcctgtctcaccagtggcctgccctctcgctcccccacccagcccacccattgaagtctccttgggccaccaaaggtggtggccatggtaccggggacttgggagagtgagacccagtggagggagcaagaggagagggatgtcgggggggtggggcacggggtaggggaaatggggtgaacggtgctggcagttcggctagatttctgtcttgtttgtttttttgttttgtttaatgtatatttttattataattattatatatgaattccaaaaaaaaaaaaaaaaaaaaa

SEQ ID NO: 11 - protein, 272 aa, Homo sapiens
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQAL

SEQ ID NO: 12 - DNA, 816 bp, Homo sapiens
atggccaaagttccagacatgtttgaagacctgaagaactgttacagtgaaaatgaagaagacagttcctccattgatcatctgtctctgaatcagaaatccttctatcatgtaagctatggcccactccatgaaggctgcatggatcaatctgtgtctctgagtatctctgaaacctctaaaacatccaagcttaccttcaaggagagcatggtggtagtagcaaccaacgggaaggttctgaagaagagacggttgagtttaagccaatccatcactgatgatgacctggaggccatcgccaatgactcagaggaagaaatcatcaagcctaggtcagcaccttttagcttcctgagcaatgtgaaatacaactttatgaggatcatcaaatacgaattcatcctgaatgacgccctcaatcaaagtataattcgagccaatgatcagtacctcacggctgctgcattacataatctggatgaagcagtgaaatttgacatgggtgcttataagtcatcaaaggatgatgctaaaattaccgtgattctaagaatctcaaaaactcaattgtatgtgactgcccaagatgaagaccaaccagtgctgctgaaggagatgcctgagatacccaaaaccatcacaggtagtgagaccaacctcctcttcttctgggaaactcacggcactaagaactatttcacatcagttgcccatccaaacttgtttattgccacaaagcaagactactgggtgtgcttggcaggggggccaccctctatcactgactttcagatactggaaaaccaggcgttg

SEQ ID NO: 13 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 14 - DNA, 1498 bp, Homo sapiens
accaaacctcttcgaggcacaaggcacaacaggctgctctgggattctcttcagccaatcttcattgctcaagtgtctgaagcagccatggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaag

SEQ ID NO: 15 - protein, 353 aa, Homo sapiens
MELTELLLVVMLLLTARLTLSSPAPPACDLRVLSKLLRDSHVLHSRLSQCPEVHPLPTPVLLPAVDFSLGEWKTQMEETKAQDILGAVTLLLEGVMAARGQLGPTCLSSLLGQLSGQVRLLLGALQSLLGTQLPPQGRTTAHKDPNAIFLSFQHLLRGKVRFLMLVGGSTLCVRRAPPTTAVPSRTSLVLTLNELPNRTSGLLETNFTASARTTGSGLLKWQQGFRAKIPGLLNQTSRSLDQIPGYLNRIHELLNGTRGLFPGPSRRTLGAPDISSGTSDTGSLPPNLQPGYSPSPTHPPTGQYTLFPLPPTLPTPVVQLHPLLPDPSAPTPTPTSPLLNTSYTHSQNLSQEG

SEQ ID NO: 16 - DNA, 555 bp, Homo sapiens
atggagctgactgaattgctcctcgtggtcatgcttctcctaactgcaaggctaacgctgtccagcccggctcctcctgcttgtgacctccgagtcctcagtaaactgcttcgtgactcccatgtccttcacagcagactgagccagtgcccagaggttcaccctttgcctacacctgtcctgctgcctgctgtggactttagcttgggagaatggaaaacccagatggaggagaccaaggcacaggacattctgggagcagtgacccttctgctggagggagtgatggcagcacggggacaactgggacccacttgcctctcatccctcctggggcagctttctggacaggtccgtctcctccttggggccctgcagagcctccttggaacccagcttcctccacagggcaggaccacagctcacaaggatcccaatgccatcttcctgagcttccaacacctgctccgaggaaaggtgcgtttcctgatgcttgtaggagggtccaccctctgcgtcaggcgggccccacccaccacagctgtccccagctga

SEQ ID NO: 17 - protein, 204 aa, Homo sapiens
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 18 - DNA, 1498 bp, Homo sapiens
ggagcctgcagcccagccccacccagacccatggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctagttgcctcccatctgcagagcttcctggaggtgtcgtaccgcgttctacgccaccttgcccagccctgagccaagccctccccatcccatgtatttatctctatttaatatttatgtctatttaagcctcatatttaaagacagggaagagcagaacggagccccaggcctctgtgtccttccctgcatttctgagtttcattctcctgcctgtagcagtgagaaaaagctcctgtcctcccatcccctggactgggaggtagataggtaaataccaagtatttattactatgactgctccccagccctggctctgcaatgggcactgggatgagccgctgtgagcccctggtcctgagggtccccacctgggacccttgagagtatcaggtctcccacgtgggagacaagaaatccctgtttaatatttaaacagcagtgttccccatctgggtccttgcacccctcactctggcctcagccgactgcacagcggcccctgcatccccttggctgtgaggcccctggacaagcagaggtggccagagctgggaggcatggccctggggtcccacgaatttgctggggaatctcgtttttcttcttaagacttttgggacatggtttgactcccgaacatcaccgacgcgtctcctgtttttctgggtggcctcgggacacctgccctgcccccacgagggtcaggactgtgactctttttagggccaggcaggtgcctggacatttgccttgctggacggggactggggatgtgggagggagcagacaggaggaatcatgtcaggcctgtgtgtgaaaggaagctccactgtcaccctccacctcttcaccccccactcaccagtgtcccctccactgtcacattgtaactgaacttcaggataataaagtgcttgcctcc

SEQ ID NO: 19 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 20 - DNA, 781 bp, Homo sapiens
acacagagagaaaggctaaagttctctggaggatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtgagaccggccagatgaggctggccaagccggggagctgctctctcatgaaacaagagctagaaactcaggatggtcatcttggagggaccaaggggtgggccacagccatggtgggagtggcctggacctgccctgggccacactgaccctgatacaggcatggcagaagaatgggaatattttatactgacagaaatcagtaatatttatatatttatatttttaaaatatttatttatttatttatttaagttcatattccatatttattcaagatgttttaccgtaataattattattaaaaatatgcttcta

SEQ ID NO: 21 - protein, 200 aa, Homo sapiens
MSPEPALSPALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 22 - DNA, 1703 bp, Homo sapiens
aaaacagcccggagcctgcagcccagccccacccagacccatggctggacctgccacccagagccccatgaagctgatgggtgagtgtcttggcccaggatgggagagccgcctgccctggcatgggagggaggctggtgtgacagaggggctggggatccccgttctgggaatggggattaaaggcacccagtgtccccgagagggcctcaggtggtagggaacagcatgtctcctgagcccgctctgtccccagccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctgtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccctgagccaagccctccccatcccatgtatttatctctatttaatatttatgtctatttaagcctcatatttaaagacagggaagagcagaacggagccccaggcctctgtgtccttccctgcatttctgagtttcattctcctgcctgtagcagtgagaaaaagctcctgtcctcccatcccctggactgggaggtagataggtaaataccaagtatttattactatgactgctccccagccctggctctgcaatgggcactgggatgagccgctgtgagcccctggtcctgagggtccccacctgggacccttgagagtatcaggtctcccacgtgggagacaagaaatccctgtttaatatttaaacagcagtgttccccatctgggtccttgcacccctcactctggcctcagccgactgcacagcggcccctgcatccccttggctgtgaggcccctggacaagcagaggtggccagagctgggaggcatggccctggggtcccacgaatttgctggggaatctcgtttttcttcttaagacttttgggacatggtttgactcccgaacatcaccgacgtgtctcctgtttttctgggtggcctcgggacacctgccctgcccccacgagggtcaggactgtgactctttttagggccaggcaggtgcctggacatttgccttgctggacggggactggggatgtgggagggagcagacaggaggaatcatgtcaggcctgtgtgtgaaaggaagctccactgtcaccctccacctcttcaccccccactcaccagtgtcccctccactgtcacattgtaactgaacttcaggataataaagtgtttgcctccaaaaaaaaaaaaaaaaaaa

SEQ ID NO: 23 - protein, 127 aa, Homo sapiens
APARSPSPSTQPWEHVNAIQEARRLLNLSRDTAAEMNETVEVISEMFDLQEPTCLQTRLELYKQGLRGSLTKLKGPLTKMASHYKQHCPPTPETSCATQTITFESLKENLKDFLLVIPFDCWEPVQE

SEQ ID NO: 24 - DNA, 384 bp, Homo sapiens
gcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccaagatggccagccactacaagcagcactgccctccaaccccggaaacttcctgtgcaacccagactatcacctttgaaagtctcaaagagaacctgaaggactttctgcttgtcatcccctttgactgctgggagccagtccaggagtga

SEQ ID NO: 25 - protein, 93 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNKRFKM

SEQ ID NO: 26 - DNA, 3524 bp, Homo sapiens
tctccgtcagccgcattgcccgctcggcgtccggcccccgacccgtgctcgtccgcccgcccgcccgcccgcccgcgccatgaacgccaaggtcgtggtcgtgctggtcctcgtgctgaccgcgctctgcctcagcgacgggaagcccgtcagcctgagctacagatgcccatgccgattcttcgaaagccatgttgccagagccaacgtcaagcatctcaaaattctcaacactccaaactgtgcccttcagattgtagcccggctgaagaacaacaacagacaagtgtgcattgacccgaagctaaagtggattcaggagtacctggagaaagctttaaacaagaggttcaagatgtgagagggtcagacgcctgaggaacccttacagtaggagcccagctctgaaaccagtgttagggaagggcctgccacagcctcccctgccagggcagggccccaggcattgccaagggctttgttttgcacactttgccatattttcaccatttgattatgtagcaaaatacatgacatttatttttcatttagtttgattattcagtgtcactggcgacacgtagcagcttagactaaggccattattgtacttgccttattagagtgtctttccacggagccactcctctgactcagggctcctgggttttgtattctctgagctgtgcaggtggggagactgggctgagggagcctggccccatggtcagccctagggtggagagccaccaagagggacgcctgggggtgccaggaccagtcaacctgggcaaagcctagtgaaggcttctctctgtgggatgggatggtggagggccacatgggaggctcacccccttctccatccacatgggagccgggtctgcctcttctgggagggcagcagggctaccctgagctgaggcagcagtgtgaggccagggcagagtgagacccagccctcatcccgagcacctccacatcctccacgttctgctcatcattctctgtctcatccatcatcatgtgtgtccacgactgtctccatggccccgcaaaaggactctcaggaccaaagctttcatgtaaactgtgcaccaagcaggaaatgaaaatgtcttgtgttacctgaaaacactgtgcacatctgtgtcttgtgtggaatattgtccattgtccaatcctatgtttttgttcaaagccagcgtcctcctctgtgaccaatgtcttgatgcatgcactgttccccctgtgcagccgctgagcgaggagatgctccttgggccctttgagtgcagtcctgatcagagccgtggtcctttggggtgaactaccttggttcccccactgatcacaaaaacatggtgggtccatgggcagagcccaagggaattcggtgtgcaccagggttgaccccagaggattgctgccccatcagtgctccctcacatgtcagtaccttcaaactagggccaagcccagcactgcttgaggaaaacaagcattcacaacttgtttttggtttttaaaacccagtccacaaaataaccaatcctggacatgaagattctttcccaattcacatctaacctcatcttcttcaccatttggcaatgccatcatctcctgccttcctcctgggccctctctgctctgcgtgtcacctgtgcttcgggcccttcccacaggacatttctctaagagaacaatgtgctatgtgaagagtaagtcaacctgcctgacatttggagtgttcccctcccactgagggcagtcgatagagctgtattaagccacttaaaatgttcacttttgacaaaggcaagcacttgtgggtttttgttttgtttttcattcagtcttacgaatacttttgccctttgattaaagactccagttaaaaaaaattttaatgaagaaagtggaaaacaaggaagtcaaagcaaggaaactatgtaacatgtaggaagtaggaagtaaattatagtgatgtaatcttgaattgtaactgttcgtgaatttaataatctgtagggtaattagtaacatgtgttaagtattttcataagtatttcaaattggagcttcatggcagaaggcaaacccatcaacaaaaattgtcccttaaacaaaaattaaaatcctcaatccagctatgttatattgaaaaaatagagcctgagggatctttactagttataaagatacagaactctttcaaaaccttttgaaattaacctctcactataccagtataattgagttttcagtggggcagtcattatccaggtaatccaagatattttaaaatctgtcacgtagaacttggatgtacctgcccccaatccatgaaccaagaccattgaattcttggttgaggaaacaaacatgaccctaaatcttgactacagtcaggaaaggaatcatttctatttctcctccatgggagaaaatagataagagtagaaactgcagggaaaattatttgcataacaattcctctactaacaatcagctccttcctggagactgcccagctaaagcaatatgcatttaaatacagtcttccatttgcaagggaaaagtctcttgtaatccgaatctctttttgctttcgaactgctagtcaagtgcgtccacgagctgtttactagggatccctcatctgtccctccgggacctggtgctgcctctacctgacactcccttgggctccctgtaacctcttcagaggccctcgctgccagctctgtatcaggacccagaggaaggggccagaggctcgttgactggctgtgtgttgggattgagtctgtgccacgtgtatgtgctgtggtgtgtccccctctgtccaggcactgagataccagcgaggaggctccagagggcactctgcttgttattagagattacctcctgagaaaaaagcttccgcttggagcagaggggctgaatagcagaaggttgcacctcccccaaccttagatgttctaagtctttccattggatctcattggacccttccatggtgtgatcgtctgactggtgttatcaccgtgggctccctgactgggagttgatcgcctttcccaggtgctacacccttttccagctggatgagaatttgagtgctctgatccctctacagagcttccctgactcattctgaaggagccccattcctgggaaatattccctagaaacttccaaatcccctaagcagaccactgataaaaccatgtagaaaatttgttattttgcaacctcgctggactctcagtctctgagcagtgaatgattcagtgttaaatgtgatgaatactgtattttgtattgtttcaagtgcatctcccagataatgtgaaaatggtccaggagaaggccaattcctatacgcagcgtgctttaaaaaataaataagaaacaactctttgagaaacaacaatttctactttgaagtcataccaatgaaaaaatgtatatgcacttataattttcctaataaagttctgtactcaaatgta

SEQ ID NO: 27 - protein, 206 aa, Homo sapiens
MSGPGTAAVALLPAVLLALLAPWAGRGGAAAPTAPNGTLEAELERRWESLVALSLARLPVAAQPKEAAVQSGAGDYLLGIKRLRRLYCNVGIGFHLQALPDGRIGGAHADTRDSLLELSPVERGVVSIFGVASRFFVAMSSKGKLYGSPFFTDECTFKEILLPNNYNAYESYKYPGMFIALSKNGKTKKGNRVSPTMKVTHFLPRL

SEQ ID NO: 28 - DNA, 1219 bp, Homo sapiens
gggagcgggcgagtaggagggggcgccgggctatatatatagcggcctcggcctcgggcgggcctggcgctcagggaggcgcgcactgctcctcagagtcccagctccagccgcgcgctttccgcccggctcgccgctccatgcagccggggtagagcccggcgcccgggggccccgtcgcttgcctcccgcacctcctcggttgcgcactcccgcccgaggtcggccgtgcgctcccgcgggacgccacaggcgcagctctgccccccagcttcccgggcgcactgaccgcctgaccgacgcacgccctcgggccgggatgtcggggcccgggacggccgcggtagcgctgctcccggcggtcctgctggccttgctggcgccctgggcgggccgagggggcgccgccgcacccactgcacccaacggcacgctggaggccgagctggagcgccgctgggagagcctggtggcgctctcgttggcgcgcctgccggtggcagcgcagcccaaggaggcggccgtccagagcggcgccggcgactacctgctgggcatcaagcggctgcggcggctctactgcaacgtgggcatcggcttccacctccaggcgctccccgacggccgcatcggcggcgcgcacgcggacacccgcgacagcctgctggagctctcgcccgtggagcggggcgtggtgagcatcttcggcgtggccagccggttcttcgtggccatgagcagcaagggcaagctctatggctcgcccttcttcaccgatgagtgcacgttcaaggagattctccttcccaacaactacaacgcctacgagtcctacaagtaccccggcatgttcatcgccctgagcaagaatgggaagaccaagaaggggaaccgagtgtcgcccaccatgaaggtcacccacttcctccccaggctgtgaccctccagaggacccttgcctcagcctcgggaagcccctgggagggcagtgcgagggtcaccttggtgcactttcttcggatgaagagtttaatgcaagagtaggtgtaagatatttaaattaattatttaaatgtgtatatattgccaccaaattatttatagttctgcgggtgtgttttttaattttctggggggaaaaaaagacaaaacaaaaaaccaactctgacttttctggtgcaacagtggagaatcttaccattggatttctttaacttgt

(19) United States
(12) Patent Application Publication
(10) Pub. No.: US 2004/0071687 A1
(43) Pub. Date: Apr. 15, 2004

(54) Adult stem cell recruitment

(76) Inventors: Shahin Rafii, Great Neck, NY (US); Beate Heissig, Tokyo (JP); Koichi Hattori, Tokyo (JP)

Correspondence Address: SCHWEGMAN, LUNDBERG, WOESSNER & KLUTH, P.A., P.O. BOX 2938, MINNEAPOLIS, MN, 55402, US

(21) Appl. No.: 10/447,315

(22) Filed: May 28, 2003

Related U.S. Application Data
(60) Provisional application No. 60/383,658, filed on May 28, 2002.

(51) Int. Cl. A61K 38/48

(52) U.S. Cl. 424/94.63

(57) ABSTRACT

The invention relates to the use of proteases to recruit stem cells from the niches they normally occupy.

### RELATED APPLICATIONS

This application claims the benefit of U.S. Provisional Application No. 60/383,658, filed May 28, 2002, which provisional application is incorporated herein by references.

### GOVERNMENT FUNDING

The invention described in this application was made with funds from the National Heart Lung and Blood Institute, Grant Number R01 HL61849-03. The United States government has certain rights in the invention.

### FIELD OF THE INVENTION

This application relates to recruitment of stem cells and/or progenitor cells from the quiescent state where they normally reside, for example, in bone marrow.

### BACKGROUND OF THE INVENTION

Stem cells are localized in a microenvironment known as the stem cell ‘niche’, where they are maintained in an undifferentiated and quiescent state. These niches are critical for regulating self-renewal and cell fate decisions, yet the molecular mechanisms governing survival and maintenance of quiescent stem cells in these specialized environments are unknown. Moreover, why and how these cells are recruited to exit these niches is not well understood.

In Drosophila, germ cells lost by normal or induced differentiation are efficiently replaced within their niches (Xie and Spradling, 2000). Some researchers believe that stromal cells provide extrinsic signals that maintain stem cell niches and regulate the repopulation of stem cells. Mice with Steel (S1/S1^{d}) mutations produce deficiencies in membrane Kit-ligand (KitL) in the tissue microenvironment, and appear to impair the proliferation and migration of spermatogonial stem cells (Ohta et al., 2000). In mammals, neurogenesis occurs within an angiogenic niche, which may provide an interface where the microenvironment of stromal cells and circulating factors influence plasticity in the adult central nervous system (Palmer et al., 2000).

Bone marrow is a major reservoir for adult organ-specific stem cells, including hematopoietic stem cells (HSCs) (Reya et al., 2001), endothelial progenitor cells (Lyden et al., 2001), neuronal stem cells and muscle stem cells (Krause et al., 2001; Blau et al., 2001). Under steady state conditions, most stem cells are in contact with bone marrow stromal cells, including osteoblasts, and are maintained in G₀ phase of cell cycle (Cheng et al., 2000), while a small fraction is in S or G₂/M phase of the cell cycle.

One of the major hurdles to studying and isolating organ-specific stem cells is that the number of stem cells in the adult bone marrow available for transplantation purposes is extremely low. Stem cells can be forced to get mobilized to the peripheral circulation by injecting the patient with cytokines (for example, G-CSF or Kit-ligand) or chemotherapeutic agents (for example, cytoxan). However, in many clinical conditions, such as aplastic anemia or myelodysplastic syndromes, these approaches fail to promote mobilization of sufficient numbers of stem cells for engraftment of the recipients, or manipulation by genetic modification. Moreover, as bone marrow contains organ-specific stem cells current mobilization approaches induce non-specific mobilization of various types of organ-specific stem cells.

Therefore, new methods to promote proliferation and mobilization a large number of organ-specific stem cells are needed to acquire sufficient numbers of stem cells that may ultimately be used for clinical purposes and for research.

### SUMMARY OF THE INVENTION

According to the invention, proteases play a key role in recruiting stem cells from a quiescent state to proliferate and differentiate. For example, stem cells within the bone marrow exist in a quiescent state, however, in MMP-9^{-/-} mice, release of sKitL and hematopoietic stem cell motility are impaired, resulting in failure of hematopoietic recovery and increased mortality. As described herein, MMP-9 enables bone marrow repopulating cells to translocate to a permissive vascular niche favoring differentiation and reconstitution of the stem cell and progenitor cell pool.

Thus, the invention is directed to a method for recruitment of adult stem cells in an animal comprising administering to the animal a protease or an activator of a protease, wherein the recruitment translocates an endogenous population of quiescent non-cycling stem cells to a permissive vascular zone in the animal so that the stem cells can proliferate, self-renew, differentiate or mobilize to a target site. This method may also be used to induce proliferation of adult stem cells in an animal.

The invention also provides a method for increasing KitL expression in an animal comprising administering to the animal a protease or an activator of a protease.

The invention further provides a method for increasing white blood cells in an animal's bloodstream comprising administering to the animal a protease or an activator or a protease.

The invention also provides a method for stimulating hematopoiesis, angiogenesis or vasculogenesis in an animal comprising administering to the animal a protease or an activator of a protease. The protease can mobilize an endogenous population of quiescent non-cycling organ-specific stem cells to a permissive vascular zone in the animal that supports proliferation or differentiation of the stem cells into hematopoietic, endothelial or other organ specific cells. Such methods are useful for replacing and repairing tissues such as blood, liver, vascular, lung and neuronal tissues.

The invention also provides a method for recruitment of adult stem cells in an animal comprising administering to the animal an effective amount of transgenic cells, wherein the transgenic cells overexpress a protease, wherein the recruitment of the stem cells translocates an endogenous population of quiescent non-cycling stem cells to a permissive vascular zone in the animal so that the stem cells proliferate, self-renew, differentiate or mobilize to a target site. The transgenic cells include a nucleic acid segment encoding the protease. The nucleic acid segment can be operably linked to a second nucleic acid segment comprising a promoter that can initiate transcription of the protease in the transgenic cells. The nucleic acid segment can further include a marker gene.

The animal may have been subjected to myelosuppressive stress.

The target site can be an injured tissue, a diseased tissue, a regenerating organ, a developing organ or bone. The target site can be blood, bone marrow, cardiac tissue, hepatic tissue, lung tissue, kidney tissue, muscle tissue, neuronal tissue, vascular tissue or a combination thereof.

The stem cells can be hematopoietic stem cells, endothelial stem cells, hepatic stem cells, neuronal stem cells, muscle stem cells or a combination thereof. The quiescent non-cycling stem cells are often in contact with bone marrow stromal cells, including osteoblasts and/or are generally maintained in a G₀ phase of cell cycle. The quiescent non-cycling stem cells can be Lin⁻Sca⁺c-Kit⁺ hematopoietics stem cells, VEGFR2⁺c-Kit⁺ endothelial stem cells, VEGFR2⁺ vascular stem cells or AC133⁺ organ-specific stem cells.

The activator can be interleukin-1, thrombopoietin, G-CSF, GMCSF, SDF-1, or fibroblast growth factor-4.

The protease can be a matrix metalloproteinase, a collagenase, a gelatinase, a stromelysin, a matrilysin, a metalloelastase, or a membrane-type matrix metalloproteinase. The protease can be a matrix metalloproteinase-1, matrix metalloproteinase-2, matrix metalloproteinase-3, matrix metalloproteinase-4, matrix metalloproteinase-4, matrix metalloproteinase-5, matrix metalloproteinase-6, matrix metalloproteinase-7, matrix metalloproteinase-8, and matrix metalloproteinase-9, matrix metalloproteinase-10, matrix metalloproteinase-11, matrix metalloproteinase-12, matrix metalloproteinase-13, matrix metalloproteinase-14 or a combination thereof. In some embodiments, the protease is matrix metalloproteinase-9.

### DESCRIPTION OF THE DRAWINGS

FIG. 1A shows that MMP-9 is induced in MMP-9^{+/+} bone marrow cells after bone marrow ablation. MMP-9^{-/-} and MMP-9^{+/+} mice received a single dose of 5-fluorouracil intravenously. Bone marrow cells obtained at different time points after 5-FU injection were cultured in serum-free medium overnight. Bone marrow cell supernatants were assayed for pro-MMP-9 by Western blot analysis. Molecular weight markers (kDa) are shown (n=5/group).

FIG. 1B shows that MMP-9 is induced in MMP-9^{+/+} bone marrow cells after bone marrow ablation. MMP-9^{-/-} and MMP-9^{+/+} mice received a single dose of 5-fluorouracil intravenously. Bone marrow cells obtained at different time points after 5-FU injection were cultured in serum-free medium overnight. Bone marrow cell supernatants were assayed for active MMP-9 by gelatin zymography. Molecular weight markers (kDa) are shown (n=5/group).

FIG. 2 illustrates an immunohistochemical analysis of pro-MMP-9 expression in bone marrow sections three days after 5-FU injection. Pro-MMP-9 was visualized as brown staining of stromal and hematopoietic elements in MMP-9^{+/+} but not MMP-9^{-/-} mice. In FIG. 2A, bone marrow sections showing stromal elements of MMP-9^{-/-} mice are shown (magnification ×100). In FIG. 2B, bone marrow sections showing hematopoietic elements of MMP-9^{-/-} mice are shown (magnification ×400). In FIG. 2C, bone marrow sections showing stromal elements of MMP-9^{+/+} mice are shown (magnification ×100). In FIG. 2D, bone marrow sections showing hematopoietic elements of MMP-9^{+/+} mice are shown (magnification ×400).

FIGS. 3A-D illustrate the delayed hematopoietic recovery and increased mortality observed in MMP-9^{-/-} mice after myelosuppression.

FIG. 3A graphically illustrates that MMP-9^{-/-} mice have substantially reduced white blood cell counts for approximately eight days longer than MMP-9^{+/+} mice after treatment with 5-fluorouracil (5-FU). MMP-9^{-/-} and MMP-9^{+/+} mice (n=16) received a single intravenous dose of 5-FU. White blood cell counts were quantified by a Neubauer chamber.

FIG. 3B graphically illustrates that MMP-9^{-/-} mice have substantially reduced survival after treatment with 5-fluoruracil, compared to MMP-9^{+/+} mice. MMP-9^{-/-} and MMP-9^{+/+} mice (n=16) received a single intravenous dose of 5-FU. Survival of 5-FU-treated mice was assessed daily (n=16).

FIG. 3C graphically illustrates that the number of S-phase Sca-1⁺ cells was higher in MMP-9^{+/+} mice as compared to MMP-9^{-/-} mice by approximately 6 days after treatment with 5-fluoruraceil. Mice (n=5) were treated with 5-FU and sacrificed at different time points. The percentage of the total number of S-phase Sca-1⁺ cells as isolated by a combination of magnetic cell isolation (MACS) and flow cytometry (FACS) is plotted. S-phase was determined by DNA content after propidium iodide staining. The total number of Sca-1⁺ cells in S-phase was higher in MMP-9^{+/+} as compared to MMP-9^{-/-} mice (2.2±0.05 vs. 1.0±0.03×10⁵/femur on day 6 and 11.5±0.3 vs. 2.1±0.04×10⁵/femur on day 10, respectively). All values are given as mean±SEM. *p<0.001, **p<0.001.

FIG. 3D graphically illustrates that the number of S-phase Lin⁻Sca-1⁺c-Kit⁺ cells was higher in MMP-9^{+/+} mice as compared to MMP-9^{-/-} mice approximately 10 days after treatment with 5-fluorouracil. Mice (n=7) were treated with 5-FU and sacrificed at different time points. The percentage of the total number of S-phase Lin⁻Sca-⁺c-Kit⁺ cells, isolated by a combination of magnetic cell isolation (MACS) and flow cytometry (FACS) is plotted. S-phase was determined by DNA content after propidium iodide staining. All values are given as mean±SEM. *p<0.001, **p<0.001.

FIGS. 4A-B illustrate that the recruitment and differentiation of hematopoietic cells were impaired in MMP-9^{-/-} mice. FIG. 4A illustrates that the frequency and distribution of myeloid and megakaryocytic precursor cells as evaluated by FACS for MMP-9^{-/-} and MMP-9^{+/+} mice. MMP-9^{-/-} and MMP-9^{+/+} mice were treated with 5-FU. The hematopoietic recovery and the frequency and distribution of myeloid and megakaryocytic precursor cells was then evaluated by FACS. Bone marrow cells obtained from either MMP-9^{-/-} or MMP-9^{+/+} mice were stained for the myeloid markers CD11b-FITC and Gr-1-PE and analyzed by FACS (FIG. 4A). The absolute number of CD11b⁺/Gr-1⁺ bone marrow cells per femur was calculated at different time points (FIG. 4B, n=6, p<0.01).

FIG. 5 illustrates that the frequency and distribution of myeloid and megakaryocytic precursor cells as evaluated by immunohistochemistry. MMP-9^{-/-} and MMP-9^{+/+} mice were treated with 5-FU. The hematopoietic recovery and the frequency and distribution of myeloid and megakaryocytic precursor cells was then evaluated by immunohistochemistry. Femurs from mice treated with 5-FU were collected, sectioned and H&E stained. Hematopoietic cell clusters were detected in close contact to osteoblasts (Osteoblastic zone, O) in the early phase of bone marrow recovery. Over time abundant clusters of proliferating hematopoietic cells were detected both in the osteoblastic zone and in the vascular-enriched zone (Vascular zone, V) in wild type animals. In contrast, there is a striking paucity of hematopoietic cell clusters in the osteoblastic and the vascular zone in 5-FU-treated MMP-9^{-/-} mice.

FIG. 6 illustrates the frequency and distribution of myeloid and megakaryocytic precursor cells as evaluated by immunohistochemistry. MMP-9^{-/-} and MMP-9^{+/+} mice were treated with 5-FU and the hematopoietic recovery and the frequency and distribution of myeloid and megakaryocytic precursor cells was evaluated by immunohistochemistry. Femurs from mice treated with 5-FU were collected at different time points, sectioned and stained for the presence of vWF (brown). The osteoblastic zone (O) and the vascular-enriched zone (V) are indicated. vWF positive megakaryocytes increase during bone marrow recovery in MMP-9^{+/+} mice, but not in MMP-9^{-/-} mice (arrows, Magnification ×100).

FIGS. 7A-D illustrate that MMP-9-mediated release of KitL enhances hematopoietic reconstitution.

FIG. 7A graphically illustrates that the plasma level of sKitL in MMP-9^{-/-} mice remains depressed while there is heightened expression of sKitL at about 6 days after 5-flourouracil treatment of MMP-9^{+/+} mice. MMP-9^{-/-} and MMP-9^{+/+} mice were injected intravenously with a single dose of 5-FU and the plasma obtained from peripheral blood (PB) was assayed for sKitL by ELISA (p<0.05 n=6/group) over a period of 10 days.

FIG. 7B graphically illustrates that MS-5 murine stromal cells treated with recombinant active MMP-9 express higher levels of sKitL than similar cells that received the matrix metalloproteinase inhibitor (MPI) CGS 27023A. Confluent MS-5 murine stromal cells, which express mKitL, were treated with recombinant active MMP-9, or the MPI (CGS 27023A) for 24 hr. *p<0.001. Control cells received no MMP-9 or CGS 27023A.

FIG. 7C graphically illustrates that delivery of sKitL via recombinant adenoviral vector (AdsKitL) increased white blood cell (WBC) counts and the number of Sca-1⁺c-Kit⁺ cells in both MMP-9^{-/-} and MMP-9^{+/+} mice. MMP-9^{-/-} and MMP-9^{+/+} mice (n=6/group) were injected intravenously on day 0 with a single dose of Ad vector encoding sKitL (AdsKitL) or no transgene (AdNull). Peripheral blood was taken at the indicated days and white blood cells were counted. PBMCs were stained for Sca-1 and c-Kit and analyzed by FACS. Injection of AdsKitL resulted in sKitL plasma levels of 5399±50 and 5126±102 pg/ml on day 5 in MMP-9^{-/-} and MMP-9^{+/+} mice, respectively.

FIG. 7D graphically illustrates that sKitL dramatically improves white blood cell (WBC) counts in MMP-9^{-/-} and effectively reverses the depression of WBC counts associated with the MMP-9^{-/-} phenotype. MMP-9^{+/+} mice MMP-9^{-/-} and MMP-9^{+/+} mice were injected with recombinant sKitL from day 3-11 after 5-FU therapy (n=10/group). WBC counts were determined at indicated time points.

FIGS. 8A-D provides copies of photomicrographs of H&E stained bone marrow sections obtained 4 days after 5-FU marrow suppression in MMP-9^{-/-} and MMP-9^{+/+} mice that received recombinant sKitL. Control MMP-9^{-/-} and MMP-9^{+/+} mice did not receive recombinant sKitL. Magnification ×200.

FIGS. 9A-9B shows that MMP-9 derived from either hematopoietic or stromal cells can reconstitute hematopoiesis after myelosuppression.

FIG. 9A illustrates the survival rate of lethally irradiated MMP-9^{-/-} and MMP-9^{+/+} mice that received bone marrow cells from either MMP-9^{-/-} or MMP-9^{+/+} mice. Lethally irradiated MMP-9^{-/-} and MMP-9^{+/+} mice were transplanted with bone marrow cells from donor MMP-9^{-/-} and MMP-9^{+/+} mice 16 days prior to 5-FU injection (n=12 in each group). After bone marrow cell engraftment a single dose of 5-FU was injected on day 0. Survival of transplanted mice was followed after a single injection of 5-FU (*p<0.05, comparing survival of −/− cells to −/− mice to the other groups).

FIG. 9B illustrates that treatment with recombinant KitL nucleic acids can increase the number of circulating endothelial precursor cells observed in the peripheral blood of MMP-9^{+/+} and MMP-9^{-/-} mice. Peripheral blood was taken from MMP-9^{-/-} and MMP-9^{+/+} mice injected with AdsKitL at indicated time points. PBMCs were plated in a colony assay and the number of CFU-C was determined (p<0.001 for number of CFC-C of MMP-9^{+/+} vs. MMP-9^{-/-} on day 4, 6 and 8).

FIG. 10 graphically illustrates that SDF-1 levels increase dramatically after MMP-9^{+/+} mice received 5-fluorouracil treatment. MMP-9^{+/+} mice were treated with a single dose of 5-FU. At indicated time points plasma was analyzed for SDF-1 by ELISA (n=6/time point).

FIGS. 11a-f shows that the bone marrow of mice treated with SDF-1, VEGF or G-CSF showed increased immunoreactive MMP-9 in stromal and hematopoietic cells. MMP-9^{-/-} and MMP-9^{+/+} mice received AdSDF-1, AdVEGF or AdNull vector by a single intravenous injection. Bone marrow sections were stained for pro-MMP-9. MMP-9^{-/-} mice treated with G-CSF served as negative control (a, b). Bone marrow sections from MMP-9^{+/+} mice after receiving AdSDF-1 (c, d), AdVEGF (e) and G-CSF (f). Magnification ×100 (a, c), ×400 (b, d-f).

FIG. 12 graphically illustrates that while human CD34⁺ cells migrate toward the chemoattractant SDF-1, treatment of CD34⁺ cells with metalloproteinase inhibitors decrease such migration. Human CD34⁺ cells were plated in Matrigel-coated transwells. Metalloproteinase inhibitors 5-phenyl-1, 10-phenanthroline and CGS 27023A were added to both chambers. Untreated cells received PBS instead (control). The chemoattractant SDF-1 was added to the lower chamber. The percentage of migrated cells was recorded. Migrated stem cells were assayed as absolute number of cobblestone forming cells (CAFC) (open bar) and long-term culture initiating cells (LTC-IC) at week 5 (n=3). The symbol * indicates a p value of p<0.05 for the migration of cells treated with or without metalloproteinase inhibitors. The insert is a gelatin zymogram of culture supernatants from human CD34⁺ cells stimulated with or without SDF-1 or VEGF in serum-free medium. Supernatants from CD34⁺ cell cultures showed gelatinolytic activity for pro-MMP-9 (92 kDa).

FIGS. 13A-F illustrate that chemo/cytokine-induced hematopoietic stem cell mobilization is impaired in MMP-9^{-/-} mice.

FIG. 13A shows that SDF-1 plasma levels can be modulated by adenoviral gene delivery of SDF-1. White blood cell (WBC) counts were increased in MMP-9^{+/+} mice that received recombinant SDF-1 nucleic acids, but were substantially unaffected in MMP-9^{-/-} mice that received recombinant SDF-1 nucleic acids. MMP-9^{-/-} and MMP-9^{+/+} mice were injected intravenously with a single dose of AdSDF-1 or AdNull on day 0 (n=10 mice in each group). Elevated chemokine levels of SDF-1 were achieved by adenoviral gene delivery of SDF-1 (bar graph insert).

FIG. 13B shows that VEG-F plasma levels and white blood cell counts can be modulated by adenoviral gene delivery of VEG-F. White blood cell (WBC) counts were increased in MMP-9^{+/+} mice that received recombinant VEG-F nucleic acids, and were slightly elevated in MMP-9^{-/-} mice that received recombinant VEG-F nucleic acids. MMP-9^{-/-} and MMP-9^{+/+} mice were injected intravenously with a single dose of AdVEGF or AdNull vector on day 0 (n=10 mice in each group). Elevated chemokine levels of VEG-F were achieved by adenoviral gene delivery of VEG-F (bar graph insert).

FIG. 13C shows that white blood cell (WBC) counts were increased in MMP-9^{+/+} mice that received recombinant G-CSF subcutaneously, and were only slightly elevated in MMP-9^{-/-} mice that received recombinant G-CSF. MMP-9^{-/-} and MMP-9^{+/+} mice were injected subcutaneously with recombinant G-CSF for days 0-5 (n=10 mice in each group).

FIG. 13D shows that the number of mobilized progenitor cells (CFU-C) is increased in MMP-9^{-/-} and MMP-9^{+/+} mice that received AdSDF-1 or AdVEGF recombinant vector or the recombinant G-CSF. MMP-9^{-/-} and MMP-9^{+/+} mice were injected intravenously with a single dose of AdSDF-1, AdVEGF or AdNull vector on day 0 or received recombinant G-CSF subcutaneously for days 0-5 (n=10 mice in each group). Mobilized PBMCs were plated in a colony assay. The number of mobilized progenitor cells (CFU-C) was determined on day 5 (AdSDF-1), on day 3 (AdVEGF) and on day 5 (G-CSF) (*p<0.05 **p<0.01).

FIG. 13E illustrates that without MMP-9, mice have reduced survival rates after lethal radiation even though G-CSF does improve the survival of both MMP-9^{-/-} and MMP-9^{+/+} mice. PBMCs were transplanted into lethally irradiated MMP-9^{-/-} and MMP-9^{+/+} syngeneic animals on day 0. Peripheral blood of MMP-9^{-/-} and MMP-9^{+/+} mice treated with or without G-CSF was obtained on day 5. Survival of transplanted recipients was monitored (n=8/group, p<0.001). As shown, G-CSF treatment of MMP-9^{+/+} mice led to 100% survival, whereas all MMP-9^{-/-} mice died by about day 18 even though they were treated with G-CSF.

FIG. 13F shows that sKitL plasma levels of MMP-9^{+/+} mice are generally higher than those of MMP-9^{-/-} mice, even after treatment with G-CSF, AdSDF-1 or AdVEGF. However, that sKitL plasma levels of MMP-9^{+/+} mice were substantially increased by treatment with G-CSF, AdSDF-1 or AdVEGF. Plasma of MMP-9^{+/+} and MMP-9^{-/-} mice was assayed for sKitL five days after G-CSF, AdNull, AdSDF-1, AdVEGF and control treatment (n=6/group, <0.03).

FIGS. 14A-D illustrate that chemokine-cytokine-mediated hematopoietic stem cells mobilization is completely blocked by metalloproteinase inhibitors. SCID mice received AdSDF-1, AdVEGF or AdNull vectors by a single intravenous injection (n=12/group). G-CSF was injected subcutaneously on a daily basis (n=12/group). A hydroxamate-based metalloproteinase inhibitor like that used in previous studies (Hattori et al., 1997) was started from day 0 and administered every 3 days subcutaneously. Pooled PBMCs were obtained on day 7 after chemokine injection in the presence/absence of metalloproteinase inhibitor. Pluripotency of mobilized cells was measured using CFU-S and bone marrow repopulating assays.

FIG. 14A illustrates that the number of spleen colony forming units in lethally irradiated mice is dramatically reduced by treatment with metalloproteinase inhibitors even when mice are treated with recombinant G-CSF, AdVEGF or AdSDF-1. Mobilized PBMCs were injected into lethally irradiated mice and spleens were harvested on day 12. Spleen colonies (CFU-S) were counted under an inverted microscope.

FIG. 14B illustrates that the survival rate of lethally irradiated mice can be improved by treatment with exogenous PBMCs and AdSDF-1, but only when no metalloproteinase inhibitor (MPI) is administered (n=12) *p<0.05.

FIG. 14C illustrates that the survival rate of lethally irradiated mice can be improved by treatment with exogenous PBMCs and VEG-F, but only when no metalloproteinase inhibitor (MPI) is administered (n=12) *p<0.05.

FIG. 14D illustrates that the survival rate of lethally irradiated mice can be improved by treatment with exogenous PBMCs and G-CSF, but only when no metalloproteinase inhibitor (MPI) is administered (n=12) *p<0.05.

FIGS. 15A-D show that endothelial progenitor cell mobilization is blocked in MMP-9^{-/-} mice. The number of circulating endothelial progenitor cells (CEPs), present in peripheral blood cultures as colony forming units of endothelial cells (CFU-EC) was determined for MMP-9^{+/+} and MMP-9^{-/-} mice three days after injection with AdVEGF, AdSDF-1, AdNull or G-CSF. Metalloproteinase inhibitors (MPI) were added to ascertain what effect metalloproteinases had on progenitor cell mobilization.

FIG. 15A graphically illustrates that the number of CEPs are dramatically reduced by metalloproteinase inhibitors, although treatment of mice with VEGF and SDF-1 can increase the number of CFU-EC observed in PBMCs collected from wild type mice. CEPs were quantified by the formation of CFU-EC and VEGFR2⁺ cells were detected by FACS. The increase in circulating CFU-EC in VEGF-treated animals correlated with the number of VEGFR2⁺cells (see also FIG. 15B).

FIG. 15B graphically illustrates that the VEGFR2 produced by PBMCs is slightly reduced by metalloproteinase inhibitors, although treatment of mice with recombinant VEGF nucleic acids can increase the number of VEGFR2⁺ cells observed. VEGFR2⁺ cells were detected by FACS. The increase in circulating CFU-EC in VEGF-treated animals correlated with the number of VEGFR2⁺ cells (see also FIG. 15A).

FIG. 15C graphically illustrates that the number of CEPs are dramatically reduced in MMP-9^{-/-} mice even after treatment with VEGF. MMP-9^{-/-} and MMP-9^{+/+} mice were injected intravenously with a single dose of AdVEGF and AdNull vector (n=6/group, *p<0.05). Mobilized PBMC were assayed for the number of CFU-EC in circulation.

FIG. 15D graphically illustrates that the percentage of VEGFR2⁺ cells in bone marrow is reduced in MMP-9^{-/-} mice even after treatment with VEGF. MMP-9^{-/-} and MMP-9^{+/+} mice were injected intravenously with a single dose of AdVEGF and AdNull vector (n=6/group, *p<0.05). Mobilized PBMC were assayed for the number of VEGFR2⁺ cells in the bone marrow on day 6 after adenoviral injection.

FIG. 16 provides a schematic diagram of the recruitment process. Under steady-state conditions quiescent c-Kit⁺ hematopoietic stem cells and CEPs reside in a niche in close contact with stromal cells, including osteoblasts. Membrane-bound cytokines such as mKitL not only convey survival signals, but also support the adhesion of stem cells to the stroma. Bone marrow ablation or chemokine/cytokine administration induce up-regulation of MMP-9 resulting in the release of sKitL. sKitL provides signals that enhances mobility of VEGFR2⁺ endothelial progenitors (CEPs) and Lin⁻Sca-1⁺c-Kit⁺ repopulating cells, translocating them into a vascular-enriched niche favoring differentiation and mobilization to the peripheral circulation.

### DETAILED DESCRIPTION OF THE INVENTION

According to the invention, localized activation or administration of proteases can recruit stem and progenitor cells from the quiescent niches that they normally occupy, for example, from the bone marrow. When present in a quiescent niche, stem cells are generally maintained in a G₀ phase of cell cycle. However, even quiescent stem cells progress through the cell cycle at a very slow rate. For example, in humans quiescent stem cells progress through the cell cycle about once every seven days. Quiescent non-cycling stem cells in bone marrow are often in contact with bone marrow stromal cells or osteoblastic cells (the osteoblastic niche). Other tissues have their own stem cell niches. Once recruited from such a quiescent niche, stem and progenitor cells become mobilized to a permissive zone. Such a permissive zone not only promotes survival of the stem cells but also promotes their differentiation. A permissive environment can therefore provide instructions for stem cell differentiation. In contrast, stem cells within a non-permissive environment would either die or fail to undergo differentiation. As the stem cells become differentiated within such a permissive zone, they can also be mobilized to target tissues. When incorporated into target tissues, differentiating stem cells can replace and repair injured or aging parts of such tissues. Hence, recruitment is a generalized term referring to the translocation of stem cells from their quiescent niches to a permissive zone, to the initiation of an increased rate of progression through the cell cycle (proliferation) and to the onset of differentiation. As described herein, localized activation or administration of proteases can facilitate recruitment of stem and progenitor and promote release of stem cell active cytokines such as Kit-ligand.

Proteases Useful for Recruiting Adult Stem and Progenitor Cells

According to the invention, proteases can be used to increase the rate of recruitment of stem cells and progenitor cells from their quiescent niches. Such proteases include, for example, metalloproteinases, collagenases, gelatinase A, gelatinase B, stromelysins, matrilysin, metalloelastase, and membrane-type matrix metalloproteinases.

The matrix metalloproteinases (MMPs) are a family of structurally similar zinc-dependent enzymes that degrade all of the major components of the extracellular matrix. MMPs include the collagenases, gelatinases A and B, the stromelysins, matrilysin, metalloelastase, and the membrane-type matrix metalloproteinases. MMPs have been linked with a range of diseases, such as arthritis, cancer, and multiple sclerosis. However, as provided herein, such proteases surprisingly have a key role in mobilizing stem cells and/or progenitor cells. Properly used, MMPs can provide beneficial renewal of stressed, injured or diseased tissues by promoting the recruitment of stem cells from the quiescent state in which they normally reside in adults.

In some embodiments, the protease used for recruiting or mobilizing stem cells can be a metalloproteinase. For example, an amino acid sequence for matrix metalloproteinase 1 is available in the NCBI database at accession number AAP36229, gi:30583961. See website at ncbi.nlm.nih.gov. This sequence for matrix metalloproteinase 1 is provided below as SEQ ID NO:1.

| | |
|---|---|
| 1 | MHSFPPLLLL LFWGVVSHSF PATLETQEQD VDLVQKYLEK |
| 41 | YYNLKNDGRQ VEKRRNSGPV VEKLKQMQEF FGLKVTGKPD |
| 81 | AETLKVMKQP RCGVPDVAQF VLTEGNPRWE QTHLTYRIEN |
| 121 | YTPDLPRADV DHAIEKAFQL WSNVTPLTFT KVSEGQADIM |
| 161 | ISFVRGDHRD NSPFDGPGGN LAHAFQPGPG IGGDAHFDED |
| 201 | ERWTNNFREY NLHRVAAHEL GHSLGLSHST DIGALMYPSY |
| 241 | TFSGDVQLAQ DDIDGIQAIY GRSQNPVQPI GPQTPKACDS |
| 281 | KLTFDAITTI RGEVMFFKDR FYMRTNPFYP EVELNFISVF |
| 321 | WPQLPNGLEA AYEFADRDEV RFFKGNKYWA VQGQNVLHGY |
| 361 | PKDIYSSFGF PRTVKHIDAA LSEENTGKTY FFVANKYWRY |
| 401 | DEYKRSMDPG YPKMIAHDFP GIGHKVDAVF MKDGFFYFFH |
| 441 | GTRQYKFDPK TKRILTLQKA NSWFNCRKNL |

A nucleotide sequence for matrix metalloproteinase 1 is available in the NCBI database at accession number BT007561, gi:30583960. See website at ncbi.nlm.nih.gov. This sequence for matrix metalloproteinase 1 is provided below as SEQ ID NO:2.

| | |
|---|---|
| 1 | ATGCACAGCT TTCCTCCACT GCTGCTGCTG CTGTTCTGGG |
| 41 | GTGTGGTGTC TCACAGCTTC CCAGCGACTC TAGAAACACA |
| 81 | AGAGCAAGAT GTGGACTTAG TCCAGAAATA CCTGGAAAAA |
| 121 | TACTACAACC TGAAGAATGA TGGGAGGCAA GTTGAAAAGC |
| 161 | GGAGAAATAG TGGCCCAGTG GTTGAAAAAT TGAAGCAAAT |
| 201 | GCAGGAATTC TTTGGGCTGA AAGTGACTGG GAAACCAGAT |
| 241 | GCTGAAACCC TGAAGGTGAT GAAGCAGCCC AGATGTGGAG |
| 281 | TGCCTGATGT GGCTCAGTTT GTCCTCACTG AGGGGAACCC |
| 321 | TCGCTGGGAG CAAACACATC TGACCTACAG GATTGAAAAT |
| 361 | TACACGCCAG ATTTGCCAAG AGCAGATGTG GACCATGCCA |
| 401 | TTGAGAAAGC CTTCCAACTC TGGAGTAATG TCACACCTCT |
| 441 | GACATTCACC AAGGTCTCTG AGGGTCAAGC AGACATCATG |
| 481 | ATATCTTTTG TCAGGGGAGA TCATCGGGAC AACTCTCCTT |
| 521 | TTGATGGACC TGGAGGAAAT CTTGCTCATG CTTTTCAACC |
| 561 | AGGCCCAGGT ATTGGAGGGG ATGCTCATTT TGATGAAGAT |
| 601 | GAAAGGTGGA CCAACAATTT CAGAGAGTAC AACTTACATC |
| 641 | GTGTTGCGGC TCATGAACTC GGCCATTCTC TTGGACTCTC |
| 681 | CCATTCTACT GATATCGGGG CTTTGATGTA CCCTAGCTAC |
| 721 | ACCTTCAGTG GTGATGTTCA GCTAGCTCAG GATGACATTG |
| 761 | ATGGCATCCA AGCCATATAT GGACGTTCCC AAAATCCTGT |
| 801 | CCAGCCCATC GGCCCACAAA CCCCAAAAGC GTGTGACAGT |
| 841 | AAGCTAACCT TTGATGCTAT AACTACGATT CGGGGAGAAG |
| 881 | TGATGTTCTT TAAAGACAGA TTCTACATGC GCACAAATCC |
| 921 | CTTCTACCCG GAAGTTGAGC TCAATTTCAT TTCTGTTTTC |
| 961 | TGGCCACAAC TGCCAAATGG GCTTGAAGCT GCTTACGAAT |
| 1001 | TTGCCGACAG AGATGAAGTC CGGTTTTTCA AAGGGAATAA |
| 1041 | GTACTGGGCT GTTCAGGGAC AGAATGTGCT ACACGGATAC |
| 1081 | CCCAAGGACA TCTACAGCTC CTTTGGCTTC CCTAGAACTG |
| 1121 | TGAAGCATAT CGATGCTGCT CTTTCTGAGG AAAACACTGG |
| 1161 | AAAAACCTAC TTCTTTGTTG CTAACAAATA CTGGAGGTAT |
| 1201 | GATGAATATA AACGATCTAT GGATCCAGGT TATCCCAAAA |
| 1241 | TGATAGCACA TGACTTTCCT GGAATTGGCC ACAAAGTTGA |
| 1281 | TGCAGTTTTC ATGAAAGATG GATTTTTCTA TTTCTTTCAT |
| 1321 | GGAACAAGAC AATACAAATT TGATCCTAAA ACGAAGAGAA |
| 1361 | TTTTGACTCT CCAGAAAGCT AATAGCTGGT TCAACTGCAG |
| 1401 | GAAAAATTTG |

An amino acid sequence for matrix metalloproteinase 2 is available in the NCBI database at accession number NP 004521, gi:11342666. See website at ncbi.nlm.nih.gov. This sequence for matrix metalloproteinase 2 is provided below as SEQ ID NO:3.

| | |
|---|---|
| 1 | MEALMARGAL TGPLPALCLL GCLLSHAAAA PSPIIKFPGD |
| 41 | VAPKTDKELA VQYLNTFYGC PKESCNLFVL KDTLKKMQKF |
| 81 | FGLPQTGDLD QNTTETMRKP RCGNPDVANY NFFPRKPKWD |
| 121 | KNQITYRIIG YTPDLDPETV DDAFAPAFQV WSDVTPLRFS |
| 161 | RIHDGEADIM INFGRWEHGD GYPFDGKDGL LAHAFAPGTG |
| 201 | VGGDSHFDDD ELWTLGEGQV VRVKYGNADG EYCKFPFLFN |
| 241 | GKEYNSCTDT GRSDGFLWCS TTYNFEKDGK YGFCPHEALF |
| 281 | TMGGNAEGQP CKFPFRFQGT SYDSCTTEGR TDGYRWCGTT |
| 321 | EDYDRDKKYG FCPETAMSTV GGNSEGAPCV FPFTFLGNKY |
| 361 | ESCTSAGRSD GKMWCATTAN YDDDRKWGFC PDQGYSLFLV |
| 401 | AAHEFGHAMG LEHSQDPGAL MAPIYTYTKN FRLSQDDIKG |
| 441 | IQELYGASPD IDLGTGPTPT LGPVTPEICK QDIVFDGIAQ |
| 431 | TRGEIFFFKD RFIWRTVTPR DKPMGPLLVA TFWPELPEKI |
| 521 | DAVYEAPQEE KAVEFAGNEY WIYSASTLER GYPKPLTSLG |
| 561 | LPPDVQRVDA AFNWSKNKKT YIFAGDKFWR YNEVKKKMDP |
| 601 | GFPKLIADAW NAIPDNLDAV VDLQGGGHSY FFKGAYYLKL |
| 641 | ENQSLKSVKF GSIKSDWLGC |

A nucleotide sequence for matrix metalloproteinase 2 is available in the NCBI database at accession number NM 004530, gi:11342665. See website at ncbi.nlm.nih.gov. This sequence for matrix metalloproteinase 2 is provided below as SEQ ID NO:4.

| | |
|---|---|
| 1 | TGTTTCCGCT GCATCCAGAC TTCCTCAGGC GGTGGCTGGA |
| 41 | GGCTGCGCAT CTGGGGCTTT AAACATACAA AGGGATTGCC |
| 81 | AGGACCTGCG GCGGCGGCGG CGGCGGCGGG GGCTGGGGCG |
| 121 | CGGGGGCCGG ACCATGAGCC GCTGAGCCGG GCAAACCCCA |
| 161 | GGCCACCGAG CCAGCGGACC CTCGGAGCGC AGCCCTGCGC |
| 201 | CGCGGACCAG GCTCCAACCA GGCGGCGAGG CGGCCACACG |
| 241 | CACCGAGCCA GCGACCCCCG GGCGACGCGC GGGGCCAGGG |
| 281 | AGCGCTACGA TGGAGGCGCT AATGGCCCGG GGCGCGCTCA |
| 321 | CGGGTCCCCT GAGGGCGCTC TGTCTCCTGG GCTGCCTGCT |
| 361 | GAGCCACGCC GCCGCCGCGC CGTCGCCCAT CATCAAGTTC |
| 401 | CCCGGCGATG TCGCCCCCAA AACGGACAAA GAGTTGGCAG |
| 441 | TGCAATACCT GAACACCTTC TATGGCTGCC CCAAGGAGAG |
| 481 | CTGCAACCTG TTTGTGCTGA AGGACACACT AAAGAAGATG |
| 521 | CAGAAGTTCT TTGGACTGCC CCAGACAGGT GATCTTGACC |
| 561 | AGAATACCAT CGAGACCATG CGGAAGCCAC GCTGCGGCAA |
| 601 | CCCAGATGTG GCCAACTACA ACTTCTTCCC TCGCAAGCCC |
| 641 | AAGTGGGACA AGAACCAGAT CACATACAGG ATCATTGGCT |
| 681 | ACACACCTGA TCTGGACCCA GAGACAGTGG ATGATGCCTT |
| 721 | TGCTCGTGCC TTCCAAGTCT GGAGCGATGT GACCCCACTG |
| 761 | CGGTTTTCTC GAATCCATGA TGGAGAGGCA GACATCATGA |
| 801 | TCAACTTTGG CCGCTGGGAG CATGGCGATG GATACCCCTT |
| 841 | TGACGGTAAG GACGGACTCC TGGCTCATGC CTTCGCCCCA |
| 881 | GGCACTGGTG TTGGGGGAGA CTCCCATTTT GATGACGATG |
| 921 | AGCTATGGAC CTTGGGAGAA GGCCAAGTGG TCCGTGTGAA |
| 961 | GTATGGCAAC GCCGATGGGG AGTACTGCAA GTTCCCCTTC |
| 1001 | TTGTTCAATG GCAAGGAGTA CAACAGCTGC ACTGATACTG |
| 1041 | GCCGCAGCGA TGGCTTCCTC TGGTGCTCCA CCACCTACAA |
| 1081 | CTTTGAGAAG GATGGCAAGT ACGGCTTCTG TCCCCATGAA |
| 1121 | GCCCTGTTCA CCATGGGCGG CAACGCTGAA GGACAGCCCT |
| 1161 | GCAAGTTTCC ATTCCGCTTC CAGGGCACAT CCTATGACAG |
| 1201 | CTGCACCACT GAGGGCCGCA CGGATGGCTA CCGCTGGTGC |
| 1241 | GGCACCACTG AGGACTACGA CCGCGACAAG AAGTATGGCT |
| 1281 | TCTGCCCTGA GACCGCCATG TCCACTGTTG GTGGGAACTC |
| 1321 | AGAAGGTGCC CCCTGTGTCT TCCCCTTCAC TTTCCTGGGC |
| 1361 | AACAAATATG AGAGCTGCAC CAGCGCCGGC CGCAGTGACG |
| 1401 | GAAAGATGTG GTGTGCGACC ACAGCCAACT ACGATGACGA |
| 1441 | CCGCAAGTGG GGCTTCTGCC CTGACCAAGG GTACAGCCTG |
| 1481 | TTCCTCGTGG CAGCCCACGA GTTTGGCCAC GCCATGGGGC |
| 1521 | TGGAGCACTC CCAAGACCCT GGGGCCCTGA TGGCACCCAT |
| 1561 | TTACACCTAC ACCAAGAACT TCCGTCTGTC CCAGGATGAC |
| 1601 | ATCAAGGGCA TTCAGGAGCT CTATGGGGCC TCTCCTGACA |
| 1641 | TTGACCTTGG CACCGGCCCC ACCCCCACAC TGGGCCCTGT |
| 1681 | CACTCCTGAG ATCTGCAAAC AGGACATTGT ATTTGATGGC |
| 1721 | ATCGCTCAGA TCCGTGGTGA GATCTTCTTC TTCAAGGACC |
| 1761 | GGTTCATTTG GCGGACTGTG ACGCCACGTG ACAAGCCCAT |
| 1801 | GGGGCCCCTG CTGGTGGCCA CATTCTGGCC TGAGCTCCCG |
| 1841 | GAAAAGATTG ATGCGGTATA CGAGGCCCCA CAGGAGGAGA |
| 1881 | AGGCTGTGTT CTTTGCAGGG AATGAATACT GGATCTACTC |
| 1921 | AGCCAGCACC CTGGAGCGAG GGTACCCCAA GCCACTGACC |
| 1961 | AGCCTGGGAC TGCCCCCTGA TGTCCAGCGA GTGGATGCCG |
| 2001 | CCTTTAACTG GAGCAAAAAC AAGAAGACAT ACATCTTTGC |
| 2041 | TGGAGACAAA TTCTGGAGAT ACAATGAGGT GAAGAAGAAA |
| 2181 | ATGGATCCTG GCTTTCCCAA GCTCATCGCA GATGCCTGGA |
| 2221 | ATGCCATCCC CGATAACCTG GATGCCGTCG TGGACCTGCA |
| 2161 | GGGCGGCGGT CACAGCTACT TCTTCAAGGG TGCCTATTAC |
| 2201 | CTGAAGCTGG AGAACCAAAG TCTGAAGAGC GTGAAGTTTG |
| 2241 | GAAGCATCAA ATCCGACTGG CTAGGCTGCT GAGCTGGCCC |
| 2281 | TGGCTCCCAC AGGCCCTTCC TCTCCACTGC CTTCGATACA |
| 2321 | CCGGGCCTGG AGAACTAGAG AAGGACCCGG AGGGGCCTGG |
| 2361 | CAGCCGTGCC TTCAGCTCTA CAGCTAATCA GCATTCTCAC |
| 2401 | TCCTACCTGG TAATTTAAGA TTCCAGAGAG TGGCTCCTCC |
| 2441 | CGGTGCCCAA GAATAGATGC TGACTGTACT CCTCCCAGGC |
| 2481 | GCCCCTTCCC CCTCCAATCC CACCAACCCT CAGAGCCACC |
| 2521 | CCTAAAGAGA TCCTTTGATA TTTTCAACGC AGCCCTGCTT |
| 2561 | TGGGCTGCCC TGGTGCTGCC ACACTTCAGG CTCTTCTCCT |
| 2601 | TTCACAACCT TCTGTGGCTC ACAGAACCCT TGGAGCCAAT |
| 2641 | GGAGACTGTC TCAAGAGGGC ACTGGTGGCC CGACAGCCTG |
| 2681 | GCACAGGGCA GTGGGACAGG GCATGGCCAG GTGGCCACTC |
| 2721 | CAGACCCCTG GCTTTTCACT GCTGGCTGCC TTAGAACCTT |
| 2761 | TCTTACATTA GCAGTTTGCT TTGTATGCAC TTTGTTTTTT |
| 2801 | TCTTTGGGTC TTGTTTTTTT TTTCCACTTA GAAATTGCAT |
| 2841 | TTCCTGACAG AAGGACTCAG GTTGTCTGAA GTCACTGCAC |
| 2881 | AGTGCATCTC AGCCCACATA GTGATGGTTC CCCTGTTCAC |
| 2921 | TCTACTTAGC ATGTCCCTAC CGAGTCTCTT CTCCACTGGA |
| 3021 | TGGAGGAAAA CCAAGCCGTG GCTTCCCGCT CAGCCCTCCC |
| 3001 | TGCCCCTCCC TTCAACCATT CCCCATGGGA AATGTCAACA |
| 3041 | AGTATGAATA AAGACACCTA CTGAGTGGC |

An amino acid sequence for matrix metalloproteinase 3 is available in the NCBI database at accession number P 08254, gi:116857. See website at ncbi.nlm.nih.gov. This sequence for matrix metalloproteinase 3 is provided below as SEQ ID NO:5.

| | |
|---|---|
| 1 | MKSLPILLLL CVAVCSAYPL DGAARGEDTS MNLVQKYLEN |
| 41 | YYDLKKDVKQ FVRRKDSGPV VKKIREMQKF LGLEVTGKLD |
| 81 | SDTLEVMRKP RCGVPDVGHF RTFPGIPKWR KTHLTYRIVN |
| 121 | YTPDLPKDAV DSAVEKALKV WEEVTPLTFS RLYEGEADIM |
| 161 | ISFAVREHGD FYPFDGPGNV LAHAYAPGPG INGDAHFDDD |
| 201 | EQWTKDTTGT NLFLVAAHEI GHSLGLFHSA NTEALMYPLY |
| 241 | HSLTDLTRFR LSQDDINGIQ SLYGPPPDSP ETPLVPTEPV |
| 281 | PPEPGTPANC DPALSFDAVS TLRGEILIFK DRHFWRKSLR |
| 321 | KLEPELHLIS SFWPSLPSGV DAAYEVTSKD LVFTFKGNQF |
| 361 | WAIRGNEVRA GYPRGIHTLG FPPTVRKIDA ATSDKEKNKT |
| 401 | YFFVEDKYWR FDEKRNSMEP GFPKQIAEDF PGIDSKIDAV |
| 441 | FEEFGFFYFF TGSSQLEFDP NAKKVTHTLK SNSWLNC |

A nucleotide sequence for matrix metalloproteinase 3 is available in the NCBI database at accession number NM 002422, gi: 13027803. See website at ncbi.nlm.nih.gov. This sequence for matrix metalloproteinase 3 is provided below as SEQ ID NO:6.

| | |
|---|---|
| 1 | ACAAGGAGGC AGGCAAGACA GCAAGGCATA GAGACAACAT |
| 41 | AGAGCTAAGT AAAGCCAGTG GAAATGAAGA GTCTTCCAAT |
| 81 | CCTACTGTTG CTGTGCGTGG CAGTTTGCTC AGCCTATCCA |
| 121 | TTGGATGGAG CTGCAAGGGG TGAGGACACC AGCATGAACC |
| 161 | TTGTTCAGAA ATATCTAGAA AACTACTACG ACCTCAAAAA |
| 201 | AGATGTGAAA CAGTTTGTTA GGAGAAAGGA CAGTGGTCCT |
| 241 | GTTGTTAAAA AAATCCGAGA AATGCAGAAG TTCCTTGGAT |
| 281 | TGGAGGTGAC GGGGAAGCTG GACTCCGACA CTCTGGAGGT |
| 321 | GATGCGCAAG CCCAGGTGTG GAGTTCCTGA TGTTGGTCAC |
| 361 | TTCAGAACCT TTCCTGGCAT CCCGAAGTGG AGGAAAACCC |
| 401 | ACCTTACATA CAGGATTGTG AATTATACAC CAGATTTGCC |
| 441 | AAAAGATGCT GTTGATTCTG CTGTTGAGAA AGCTCTGAAA |
| 481 | GTCTGGGAAG AGGTGACTCC ACTCACATTC TCCAGGCTGT |
| 521 | ATGAAGGAGA GGCTGATATA ATGATCTCTT TTGCAGTTAG |
| 561 | AGAACATGGA GACTTTTACC CTTTTGATGG ACCTGGAAAT |
| 601 | GTTTTGGCCC ATGCCTATGC CCCTGGGCCA GGGATTAATG |
| 641 | GAGATGCCCA CTTTGATGAT GATGAACAAT GGACAAAGGA |
| 681 | TACAACAGGG ACCAATTTAT TTCTCGTTGC TGCTCATGAA |
| 721 | ATTGGCCACT CCCTGGGTCT CTTTCACTCA GCCAACACTG |
| 761 | AAGCTTTGAT GTACCCACTC TATCACTCAC TCACAGACCT |
| 801 | GACTCGGTTC CGCCTGTCTC AAGATGATAT AAATGGCATT |
| 841 | CAGTCCCTCT ATGGACCTCC CCCTGACTCC CCTGAGACCC |
| 881 | CCCTGGTACC CACGGAACCT GTCCCTCCAG AACCTGGGAC |
| 921 | GCCAGCCAAC TGTGATCCTG CTTTGTCCTT TGATGCTGTC |
| 961 | AGCACTCTGA GGGGAGAAAT CCTGATCTTT AAAGACAGGC |
| 1001 | ACTTTTGGCG CAAATCCCTC AGGAAGCTTG AACCTGAATT |
| 1041 | GCATTTGATC TCTTCATTTT GGCCATCTCT TCCTTCAGGC |
| 1081 | GTGGATGCCG CATATGAAGT TACTAGCAAG GACCTCGTTT |
| 1121 | TCATTTTTAA AGGAAATCAA TTCTGGGCCA TCAGAGGAAA |
| 1161 | TGAGGTACGA GCTGGATACC CAAGAGGCAT CCACACCCTA |
| 1201 | GGTTTCCCTC CAACCGTGAG GAAAATCGAT GCAGCCATTT |
| 1241 | CTGATAAGGA AAAGAACAAA ACATATTTCT TTGTAGAGGA |
| 1281 | CAAATACTGG AGATTTGATG AGAAGAGAAA TTCCATGGAG |
| 1321 | CCAGGCTTTC CCAAGCAAAT AGCTGAAGAC TTTCCAGGGA |
| 1361 | TTGACTCAAA GATTGATGCT GTTTTTGAAG AATTTGGGTT |
| 1401 | CTTTTATTTC TTTACTGGAT CTTCACAGTT GGAGTTTGAC |
| 1441 | CCAAATGCAA AGAAAGTGAC ACACACTTTG AAGAGTAACA |
| 1481 | GCTGGCTTAA TTGTTGAAAG AGATATGTAG AAGGCACAAT |
| 1521 | ATGGGCACTT TAAATGAAGC TAATAATTCT TCACCTAAGT |
| 1561 | CTCTGTGAAT TGAAATGTTC GTTTTCTCCT GCCTGTGCTG |
| 1601 | TGACTCGAGT CACACTCAAG GGAACTTGAG CGTGAATCTG |
| 1641 | TATCTTGCCG GTCATTTTTA TGTTATTACA GGGCATTCAA |
| 1681 | ATGGGCTGcT GCTTAGCTTG CACCTTGTCA CATAGAGTGA |
| 1721 | TCTTTCCCAA GAGAAGGGGA AGCACTCGTG TGCAACAGAC |
| 1761 | AAGTGACTGT ATCTGTGTAG ACTATTTGCT TATTTAATAA |
| 1801 | AGACGATTTG TCAGTTGTTT T |

An amino acid sequence for matrix metalloproteinase 9 is available in the NCBI database at accession number NP 004985, gi:4826836. See website at ncbi.nlm.nih.gov. This sequence for matrix metalloproteinase 9 is provided below as SEQ ID NO:7.

| | |
|---|---|
| 1 | MSLWQPLVLV LLVLGCCFAA PRQRQSTLVL FPGDLRTNLT |
| 41 | DRQLAEEYLY RYGYTRVAEM RGESKSLGPA LLLLQKQLSL |
| 81 | PETGELDSAT LKAMRTPRCG VPDLGRFQTF EGDLKWHHHN |
| 121 | ITYWIQNYSE DLPRAVIDDA FAPAFALWSA VTPLTFTRVY |
| 161 | SRDADIVIQF GVAEHGDGYP FDGKDGLLAH AFPPGPGIQG |
| 201 | DAHFDDDELW SLGKGVVVPT RFGNADGAAC HFPFIFEGRS |
| 241 | YSACTTDGRS DGLPWCSTTA NYDTDDRFGF CPSERLYTRD |
| 281 | GNADGKPCQF PFIFQGQSYS ACTTDGRSDG YRWCATTANY |
| 321 | DRDKLFGFCP TPADSTVMGG NSAGELCVFP FTFLGKEYST |
| 361 | CTSEGRGDGR LWCATTSNFD SDKKWGFCPD QGYSLFLVAA |
| 401 | HEFGHALGLD HSSVPEALMY PMYRFTEGPP LHKDDVNGIR |
| 441 | HLYGPRPEPE PRPPTTTTPQ PTAPPTVCPT GPPTVHPSER |
| 481 | PTAGPTGPPS AGPTGPPTAG PSTATTVPLS PVDDACNVNI |
| 521 | FDAIAEIGNQ LYLFKDGKYW RFSEGRGSRP QGPFLIADKW |
| 561 | PALPRKLDSV FEEPLSKKLF FFSGRQVWVY TGASVLGPRR |
| 601 | LDKLGLGADV AQVTGALRSG RGKMLLFSGR RLWRFDVKAQ |
| 641 | MVDPRSASEV DRMFPGVPLD THDVFQYREK AYFCQDRFYW |
| 681 | RVSSRSELNQ VDQVGYVTYD ILQCPED |

A nucleotide sequence for matrix metalloproteinase 9 is available in the NCBI database at accession number NM 004994, gi:4826835. See website at ncbi.nlm.nih.gov. This sequence for matrix metalloproteinase 9 is provided below as SEQ ID NO:8.

| | |
|---|---|
| 1 | AGACACCTCT GCCCTCACCA TGAGCCTCTG GCAGCCCCTG |
| 41 | GTCCTGGTGC TCCTGGTGCT GGGCTGCTGc TTTGCTGCCC |
| 81 | CCAGACAGCG CCAGTCCACC CTTGTGCTCT TCCCTGGAGA |
| 121 | CCTGAGAACC AATCTCACCG ACAGGCAGCT GGCAGAGGAA |
| 161 | TACCTGTACC GCTATGGTTA CACTCGGGTG GCAGAGATGC |
| 201 | GTGGAGAGTC GAAATCTCTG GGGCCTGCGC TGCTGCTTCT |
| 241 | CCAGAAGCAA CTGTCCCTGC CCGAGACCGG TGAGCTGGAT |
| 281 | AGCGCCACGC TGAAGGCCAT GCGAACCCCA CGGTGCGGGG |
| 321 | TCCCAGACCT GGGCAGATTC CAAACCTTTG AGGGCGACCT |
| 361 | CAAGTGGCAC CACCACAACA TCACCTATTG GATCCAAAAC |
| 401 | TACTCGGAAG ACTTGCCGCG GGCGGTGATT GACGACGCCT |
| 441 | TTGCCCGCGC CTTCGCACTG TGGAGCGCGG TGACGCCGCT |
| 481 | CACCTTCACT CGCGTGTACA GCCGGGACGC AGACATCGTC |
| 521 | ATCCAGTTTG GTGTCGCGGA GCACGGAGAC GGGTATCCCT |
| 561 | TCGACGGGAA GGACGGGCTC CTGGCACACG CCTTTCCTCC |
| 601 | TGGCCCCGGC ATTCAGGGAG ACGCCCATTT CGACGATGAC |
| 641 | GAGTTGTGGT CCCTGGGCAA GGGCGTCGTG GTTCCAACTC |
| 681 | GGTTTGGAAA CGCAGATGGC GCGGCCTGCC ACTTCCCCTT |
| 721 | CATCTTCGAG GGCCGCTCCT ACTCTGCCTG CACCACCGAC |
| 761 | GGTCGCTCCG ACGGCTTGCC CTGGTGCAGT ACCACGGCCA |
| 801 | ACTACGACAC CGACGACCGG TTTGGCTTCT GCCCCAGCGA |
| 841 | GAGACTCTAC ACCCGGGACG GCAATGCTGA TGGGAAACCC |
| 881 | TGCCAGTTTC CATTCATCTT CCAAGGCCAA TCCTACTCCG |
| 921 | CCTGCACCAC GGACGGTCGC TCCGACGGCT ACCGCTGGTG |
| 961 | CGCCACCACC GCCAACTACG ACCGGGACAA GCTCTTCGGC |
| 1001 | TTCTGCCCGA CCCGAGCTGA CTCGACGGTG ATGGGGGGCA |
| 1021 | ACTCGGCGGG GGAGCTGTGC GTCTTCCCCT TCACTTTCCT |
| 1081 | GGGTAAGGAG TACTCGACCT GTACCAGCGA GGGCCGCGGA |
| 1121 | GATGGGCGCC TCTGGTGCGC TACCACCTCG AACTTTGACA |
| 1161 | GCGACAAGAA GTGGGGCTTC TGCCCGGACC AAGGATACAG |
| 1201 | TTTGTTCCTC GTGGCGGCGC ATGAGTTCGG CCACGCGCTG |
| 1241 | GGCTTAGATC ATTCCTCAGT GCCGGAGGCG CTCATGTACC |
| 1281 | CTATGTACCG CTTCACTGAG GGGCCCCCCT TGCATAAGGA |
| 1321 | CGACGTGAAT GGCATCCGGC ACCTCTATGG TCCTCGCCCT |
| 1361 | GAACCTGAGC CACGGCCTCC AACCACCACC ACACCGCAGC |
| 1401 | CCACGGCTCC CCCGACGGTC TGCCCCACCG GACCCCCCAC |
| 1441 | TGTCCACCCC TCAGAGCGCC CCACAGCTGG CCCCACAGGT |
| 1481 | CCCCCCTCAG CTGGCCCCAC AGGTCCCCCC ACTGCTGGCC |
| 1521 | CTTCTACGGC CACTACTGTG CCTTTGAGTC CGGTGGACGA |
| 1561 | TGCCTGCAAC GTGAACATCT TCGACGCCAT CGCGGAGATT |
| 1601 | GGGAACCAGC TGTATTTGTT CAAGGATGGG AAGTACTGGC |
| 1641 | GATTCTCTGA GGGCAGGGGG AGCCGGCCGC AGGGCCCCTT |
| 1681 | CCTTATCGCC GACAAGTGGC CCGCGCTGCC CCGCAAGCTG |
| 1721 | GACTCGGTCT TTGAGGAGCC GCTCTCCAAG AAGCTTTTCT |
| 1761 | TCTTCTCTGG GCGCCAGGTG TGGGTGTACA CAGGCGCGTC |
| 1801 | GGTGCTGGGC CCGAGGCGTC TGGACAAGCT GGGCCTGGGA |
| 1841 | GCCGACGTGG CCCAGGTGAC CGGGGCCCTC CGGAGTGGCA |
| 1881 | GGGGGAAGAT GCTGCTGTTC AGCGGGCGGC GCCTCTGGAG |
| 1921 | GTTCGACGTG AAGGCGCAGA TGGTCGATCC CCGGAGCGCC |
| 1961 | AGCGAGGTGG ACCGGATGTT CCCCGGGGTG CCTTTGGACA |
| 2001 | CGCACCACGT CTTCCAGTAC CGAGAGAAAG CCTATTTCTG |
| 2041 | CCAGGACCGC TTCTACTGGC GCGTGAGTTC CCGGAGTGAG |
| 2081 | TTGAACCAGG TGGACCAAGT GGGCTACGTG ACCTATGACA |
| 2121 | TCCTGCAGTG CCCTGAGGAC TAGGGCTCCC GTCCTGCTTT |
| 2161 | GCAGTGCCAT GTAAATCCCC ACTGGGACCA ACCCTGGGGA |
| 2201 | AGGAGCCAGT TTGCCGGATA CAAACTGGTA TTCTGTTCTG |
| 2241 | GAGGAAAGGG AGGAGTGGAG GTGGGCTGGG CCCTCTCTTC |
| 2281 | TCACCTTTGT TTTTTGTTGG AGTGTTTCTA ATAAACTTGG |
| 2321 | ATTCTCTAAC CTTT |

An amino acid sequence for matrix metalloproteinase 14 is available in the NCBI database at accession number NP 004986, gi:4826834. See website at ncbi.nlm.nih.gov. This sequence for matrix metalloproteinase 14 is provided below as SEQ ID NO:9.

| | |
|---|---|
| 1 | MSPAPRPPRC LLLPLLTLGT ALASLGSAQS SSFSPEAWLQ |
| 41 | QYGYLPPGDL RTHTQRSPQS LSAAIAAMQK FYGLQVTGKA |
| 81 | DADTMKANRR PRCGVPDKFG AEIKANVRRK RYAIQGLKWQ |
| 121 | HNEITFCIQN YTPKVGEYAT YEAIRKAFRV WESATPLRFR |
| 161 | EVPYAYIREG HEKQADTMTF FAEGFHGDST PFDGEGGFLA |
| 201 | HAYFPGPNIG GDTHFDSAEP WTVRNEDLNG NDIFLVAVHE |
| 241 | LGHALGLEHS SDPSAIMAPF YQWMDTENFV LPDDDRRGIQ |
| 281 | QLYGGESGFP TKMPPQPRTT SRPSVPDKPK NPTYGPNICD |
| 321 | GNFDTVAMLR GEMFVFKERW FWRVRNNQVM DGYPMPIGQF |
| 361 | WRGLPASINT AYERKDGKFV FFKGDKHWVF DEASLEPGYP |
| 401 | KHIKELGRGL PTDKIDAALF WMPNGKTYFF RGNKYYRFNE |
| 441 | ELPAVDSEYP KNTKVWEGIP ESPRGSFMGS DEVFTYFYKG |
| 481 | NKYWKFNNQK LKVEPGYPKS ALRDWMGCPS GGRPDEGTEE |
| 521 | ETEVTTIEVD EEGGGAVSAA AVVLPVLLLL LVLAVGLAVF |
| 561 | FFRRHGTPRR LLYCQRSLLD KV |

A nucleotide sequence for matrix metalloproteinase 14 is available in the NCBI database at accession number NM 004995, gi: 13027797. See website at ncbi.nlm.nih.gov. This sequence for matrix metalloproteinase 14 is provided below as SEQ ID NO:10.

| | |
|---|---|
| 1 | CAGACCCCAG TTCGCCGACT AAGCAGAAGA AAGATCAAAA |
| 41 | ACCGGAAAAG AGGAGAAGAG CAAACAGGCA CTTTGAGGAA |
| 81 | CAATCCCCTT TAACTCCAAG CCGACAGCGG TCTAGGAATT |
| 121 | CAAGTTCAGT GCCTACCGAA GACAAAGGCG CCCCGAGGGA |
| 161 | GTGGCGGTGC GACCCCAGGG CGTGGGCCCG GCCGCGGAGC |
| 201 | CCACACTGCC CGGCTGACCC GGTGGTCTCG GACCATGTCT |
| 241 | CCCGCCCCAA GACCCCCCCG TTGTCTCCTG CTCCCCCTGC |
| 281 | TCACGCTCGG CACCGCGCTC GCCTCCCTCG GCTCGGCCCA |
| 321 | AAGCAGCAGC TTCAGCCCCG AAGCCTGGCT ACAGCAATAT |
| 361 | GGCTACCTGC CTCCCGGGGA CCTACGTACC CACACACAGC |
| 401 | GCTCACCCCA GTCACTCTCA GCGGCCATCG CTGCCATGCA |
| 441 | GAAGTTTTAC GGCTTGCAAG TAACAGGCAA AGCTGATGCA |
| 481 | GACACCATGA AGGCCATGAG GCGCCCCCGA TGTGGTGTTC |
| 521 | CAGACAAGTT TGGGGCTGAG ATCAAGGCCA ATGTTCGAAG |
| 561 | GAAGCGCTAC GCCATCCAGG GTCTCAAATG GCAACATAAT |
| 601 | GAAATCACTT TCTGCATCCA GAATTACACC CCCAAGGTGG |
| 641 | GCGAGTATGC CACATACGAG GCCATTCGCA AGGCGTTCCG |
| 681 | CGTGTGGGAG AGTGCCACAC CACTGCGCTT CCGCGAGGTG |
| 721 | CCCTATGCCT ACATCCGTGA GGGCCATGAG AAGCAGGCCG |
| 761 | ACATCATGAT CTTCTTTGCC GAGGGCTTCC ATGGCGACAG |
| 801 | CACGCCCTTC GATGGTGAGG GCGGCTTCCT GGCCCATGCC |
| 841 | TACTTCCCAG GCCCCAACAT TGGAGGAGAC ACCCACTTTG |
| 881 | ACTCTGCCGA GCCTTGGACT GTCAGGAATG AGGATCTGAA |
| 921 | TGGAAATGAC ATCTTCCTGG TGGCTGTGCA CGAGCTGGGC |
| 961 | CATGCCCTGG GGCTCGAGCA TTCCAGTGAC CCCTCGGCCA |
| 1001 | TCATGGCACC CTTTTACCAG TGGATGGACA CGGAGAATTT |
| 1041 | TGTGCTGCCC GATGATGACC GCCGGGGCAT CCAGCAACTT |
| 1081 | TATGGGGGTG AGTCAGGGTT CCCCACCAAG ATGCCCCCTC |
| 1121 | AACCCAGGAC TACCTCCCGG CCTTCTGTTC CTGATAAACC |
| 1161 | CAAAAACCCC ACCTATGGGC CCAACATCTG TGACGGGAAC |
| 1201 | TTTGACACCG TGGCCATGCT CCGAGGGGAG ATGTTTGTCT |
| 1241 | TCAAGGAGCG CTGGTTCTGG CGGGTGAGGA ATAACCAAGT |
| 1281 | GATGGATGGA TACCCAATGC CCATTGGCCA GTTCTGGCGG |
| 1321 | GGCCTGCCTG CGTCCATCAA CACTGCCTAC GAGAGGAAGG |
| 1361 | ATGGCAAATT CGTCTTCTTC AAAGGAGACA AGCATTGGGT |
| 1401 | GTTTGATGAG GCGTCCCTGG AACCTGGCTA CCCCAAGCAC |
| 1441 | ATTAAGGAGC TGGGCCGAGG GCTGCCTACC GACAAGATTG |
| 1481 | ATGCTGCTCT CTTCTGGATG CCCAATGCAA AGACCTACTT |
| 1521 | CTTCCGTGGA AACAAGTACT ACCGTTTCAA CGAAGAGCTC |
| 1561 | AGGGCAGTGG ATAGCGAGTA CCCCAAGAAC ATCAAAGTCT |
| 1601 | GGGAAGGGAT CCCTGAGTCT CCCAGAGGGT CATTCATGGG |
| 1641 | CAGCGATGAA GTCTTCACTT ACTTCTACAA GGGGAACAAA |
| 1681 | TACTGGAAAT TCAACAACCA GAAGCTGAAG GTAGAACCGG |
| 1721 | GCTACCCCAA GTCAGCCCTG AGGGACTGGA TGGGCTGCCC |
| 1761 | ATCGGGAGGC CGGCCGGATG AGGGGACTGA GGAGGAGACG |
| 1801 | GAGGTGATCA TCATTGAGGT GGACGAGGAG GGCGGCGGGG |
| 1841 | CGGTGAGCGC GGCTGCCGTG GTGCTGCCCG TGCTGCTGCT |
| 1881 | GCTCCTGGTG CTGGCGGTGG GCCTTGCAGT CTTCTTCTTC |
| 1921 | AGACGCCATG GGACCCCCAG GCGACTGCTC TACTGCCAGC |
| 1961 | GTTCCCTGCT GGACAAGGTC TGACGCCCAC CGCCGGCCCG |
| 2001 | CCCACTCCTA CCACAAGGAC TTTGCCTCTG AAGGCCAGTG |
| 2041 | GCAGCAGGTG GTGGTGGGTG GGCTGCTCCC ATCGTCCCGA |
| 2081 | GCCCCCTCCC CGCAGCCTCC TTGCTTCTCT CTGTCCCCTG |
| 2121 | GCTGGCCTCC TTCACCCTGA CCGCCTCCCT CCCTCCTGCC |
| 2161 | CCGGCATTGC ATCTTCCCTA GATAGGTCCC CTGAGGGCTG |
| 2201 | AGTGGGAGGG CGGCCCTTTC CAGCCTCTGC CCCTCAGGGG |
| 2241 | AACCCTGTAG CTTTGTGTCT GTCCAGCCCC ATCTGAATGT |
| 2281 | GTTGGGGGCT CTGCACTTGA AGGCAGGACC CTCAGACCTC |
| 2321 | GCTGGTAAAG GTCAAATGGG GTCATCTGCT CCTTTTCCAT |
| 2361 | CCCCTGACAT ACCTTAACCT CTGAACTCTG ACCTCAGGAG |
| 2401 | GCTCTGGGCA CTCCAGCCCT GAAAGCCCCA GGTGTACCCA |
| 2441 | ATTGGCAGCC TCTCACTACT CTTTCTGGCT AAAAGGAATC |
| 2481 | TAATCTTGTT GAGGGTAGAG ACCCTGAGAC AGTGTGAGGG |
| 2521 | GGTGGGGACT GCCAAGCCAC CCTAAGACCT TGGGAGGAAA |
| 2561 | ACTCAGAGAG GGTCTTCGTT GCTCAGTCAG TCAAGTTCCT |
| 2601 | CGGAGATCTG CCTCTGCCTC ACCTACCCCA GGGAACTTCC |
| 2641 | AAGGAAGGAG CCTGAGCCAC TGGGGACTAA GTGGGCAGAA |
| 2681 | GAAACCCTTG GCAGCCCTGT GCCTCTCGAA TGTTAGCCTT |
| 2721 | GGATGGGGCT TTCACAGTTA GAAGAGCTGA AACCAGGGGT |
| 2761 | GCAGCTGTCA GGTAGGGTGG GGCCGGTGGG AGAGGCCCGG |
| 2801 | GTCAGAGCCC TGGGGGTGAG CCTGAAGGCC ACAGAGAAAG |
| 2841 | AACCTTGCCC AAACTCAGGC AGCTGGGGCT GAGGCCCAAA |
| 2881 | GGCAGAACAG CCAGAGGGGG CAGGAGGGGA CCAAAAAGGA |
| 2921 | AAATGAGGAC GTGCAGCAGC ATTGGAAGGC TGGGGCCGGG |
| 2961 | CAGGCCAGGC CAAGCCAAGC AGGGGGCCAC AGGGTGGGCT |
| 3001 | GTGGAGCTCT CAGGAAGGGC CCTGAGGAAG GCACACTTGC |
| 3041 | TCCTGTTGGT CCCTGTCCTT GCTGCCCAGG CAGCGTGGAG |
| 3081 | GGGAAGGGTA GGGCAGCCAG AGAAAGGAGC AGAGAAGGCA |
| 3121 | CACAAACGAG GAATGAGGGG CTTCACGAGA GGCCACAGGG |
| 3161 | CCTGGCTGGC CACGCTGTCC CGGCCTGCTC ACCATCTCAG |
| 3201 | TGAGGGGCAG GAGCTGGGGC TCGCTTAGGC TGGGTCCACG |
| 3241 | CTTCCCTGGT GCCAGCACCC CTCAAGCCTG TCTCACCAGT |
| 3281 | GGCCTGCCCT CTCGCTCCCC CACCCAGCCC ACCCATTGAA |
| 3321 | GTCTCCTTGG GCCACCAAAG GTGGTGGCCA TGGTACCGGG |
| 3361 | GACTTGGGAG AGTGAGACCC AGTGGAGGGA GCAAGAGGAG |
| 3401 | AGGGATGTCG GGGGGGTGGG GCACGGGGTA GGGGAAATGG |
| 3441 | GGTGAACGGT GCTGGCAGTT CGGCTAGATT TCTGTCTTGT |
| 3481 | TTGTTTTTTT GTTTTGTTTA ATGTATATTT TTATTATAAT |
| 3521 | TATTATATAT GAATTCCAAA AAAAAAAAAA |

Activators of Proteases

According to the invention, any available activator of a protease can be utilized to stimulate protease expression or activity. In general, activators that optimally promote recruitment and/or mobilization of stem or progenitor cells are selected for use. Examples of activators of proteases such as matrix metalloproteinase-9 include interleukin-1, thrombopoietin, G-CSF, GMCSF, sdf-1, or fibroblast growth factor-4 (FGF-4).

In one embodiment, the protease can be activated by interleukin-1. An amino acid sequence for alpha human interleukin-1 is available in the NCBI database at accession number AAP36415, gi:30584333. See website at ncbi.nlm.nih.gov. This sequence for alpha human interleukin-1 is provided below as SEQ ID NO:11.

| | |
|---|---|
| 1 | MAKVPDMFED LKNCYSENEE DSSSIDHLSL NQKSFYHVSY |
| 41 | GPLHEGCMDQ SVSLSTSETS KTSKLTFKES MVVVATNGKV |
| 81 | LKKRRLSLSQ SITDDDLEAI ANDSEEEIIK PRSAPFSFLS |
| 121 | NVKYNFMRTI KYEFILNDAL NQSIIPANDQ YLTAAALHNL |
| 161 | DEAVKFDMGA YKSSKDDAKI TVILRISKTQ LYVTAQDEDQ |
| 201 | PVLLKEMPEI PKTITGSETN LLFFWETHGT KNYFTSVAHP |
| 241 | NLFIATKQDY WVCLAGGPPS ITDFQILENQ AL |

A nucleotide sequence for alpha human interleukin-1 is available in the NCBI database at accession number BT007747, gi:30584332. See website at ncbi.nlm.nih.gov. This sequence for alpha human interleukin-1 is provided below as SEQ ID NO:12.

| | |
|---|---|
| 1 | ATGGCCAAAG TTCCAGACAT GTTTGAAGAC CTGAAGAACT |
| 41 | GTTACAGTGA AAATGAAGAA GACAGTTCCT CCATTGATCA |
| 81 | TCTGTCTCTG AATCAGAAAT CCTTCTATCA TGTAAGCTAT |
| 121 | GGCCCACTCC ATGAAGGCTG CATGGATCAA TCTGTGTCTC |
| 161 | TGAGTATCTC TGAAACCTCT AAAACATCCA AGCTTACCTT |
| 201 | CAAGGAGAGC ATGGTGGTAG TAGCAACCAA CGGGAAGGTT |
| 241 | CTGAAGAAGA GACGGTTGAG TTTAAGCCAA TCCATCACTG |
| 281 | ATGATGACCT GGAGGCCATC GCCAATGACT CAGAGGAAGA |
| 321 | AATCATCAAG CCTAGGTCAG CACCTTTTAG CTTCCTGAGC |
| 361 | AATGTGAAAT ACAACTTTAT GAGGATCATC AAATACGAAT |
| 401 | TCATCCTGAA TGACGCCCTC AATCAAAGTA TAATTCGAGC |
| 441 | CAATGATCAG TACCTCACGG CTGCTGCATT ACATAATCTG |
| 481 | GATGAAGCAG TGAAATTTGA CATGGGTGCT TATAAGTCAT |
| 521 | CAAAGGATGA TGCTAAAATT ACCGTGATTC TAAGAATCTC |
| 561 | APAAACTCAA TTGTATGTGA CTGCCCAAGA TGAAGACCAA |
| 601 | CCAGTGCTGC TGAAGGAGAT GCCTGAGATA CCCAAAACCA |
| 641 | TCACAGGTAG TGAGACCAAC CTCCTCTTCT TCTGGGAAAC |
| 681 | TCACGGCACT AAGAACTATT TCACATCAGT TGCCCATCCA |
| 721 | AACTTGTTTA TTGCCACAAA GCAAGACTAC TGGGTGTGCT |
| 761 | TGGCAGGGGG GCCACCCTCT ATCACTGACT TTCAGATACT |
| 801 | GGAAAACCAG GCGTTG |

An amino acid sequence for beta human interleukin-1 is available in the NCBI database at accession number AAP35877, gi:30583265. See website at ncbi.nlm.nih.gov. This sequence for beta human interleukin-1 is provided below as SEQ ID NO:13.

| | |
|---|---|
| 1 | MAEVPELASE MMAYYSGNED DLFFEADGPK QMKCSFQDLD |
| 41 | LCPLDGGIQL RISDHHYSKG FRQAASVVVA MDKLRKMLVP |
| 81 | CPQTFQENDL STFFPFIFEE EPTFFDTWDN EAYVHDAPVR |
| 121 | SLNCTLRDSQ QKSLVMSGPY ELKALHLQGQ DMEQQVVFSM |
| 161 | SFVQGEESND KIPVALGLKE KNLYLSCVLK DDKPTLQLES |
| 201 | VDPKNYPKKK MEKRFVFNKT ETNNKLEFES AQFPNWYIST |
| 241 | SQAENMPVFL GGTKGGQDIT DFTMQFVSS |

A nucleotide sequence for beta human interleukin-1 is available in the NCBI database at accession number NM 000576, gi:27894305. See website at ncbi.nlm.nih.gov. This sequence for beta human interleukin-1 is provided below as SEQ ID NO:14.

| | |
|---|---|
| 1 | ACCAAACCTC TTCGAGGCAC AAGGCACAAC AGGCTGCTCT |
| 41 | GGGATTCTCT TCAGCCAATC TTCATTGCTC AAGTGTCTGA |
| 81 | AGCAGCCATG GCAGAAGTAC CTGAGCTCGC CAGTGAAATG |
| 121 | ATGGCTTATT ACAGTGGCAA TGAGGATGAC TTGTTCTTTG |
| 161 | AAGCTGATGG CCCTAAACAG ATGAAGTGCT CCTTCCAGGA |
| 201 | CCTGGACCTC TGCCCTCTGG ATGGCGGCAT CCAGCTACGA |
| 241 | ATCTCCGACC ACCACTACAG CAAGGGCTTC AGGCAGGCCG |
| 281 | CGTCAGTTGT TGTGGCCATG GACAAGCTGA GGAAGATGCT |
| 321 | GGTTCCCTGC CCACAGACCT TCCAGGAGAA TGACCTGAGC |
| 361 | ACCTTCTTTC CCTTCATCTT TGAAGAAGAA CCTATCTTCT |
| 401 | TCGACACATG GGATAACGAG GCTTATGTGC ACGATGCACC |
| 441 | TGTACGATCA CTGAACTGCA CGCTCCGGGA CTCACAGCAA |
| 481 | AAIAAGCTTGG TGATGTCTGG TCCATATGAA CTGAAAGCTC |
| 521 | TCCACCTCCA GGGACAGGAT ATGGAGCAAC AAGTGGTGTT |
| 561 | CTCCATGTCC TTTGTACAAG GAGAAGAAAG TAATGACAAA |
| 601 | ATACCTGTGG CCTTGGGCCT CAAGGAAAAG AATCTGTACC |
| 641 | TGTCCTGCGT GTTGAAAGAT GATAAGCCCA CTCTACAGCT |
| 681 | GGAGAGTGTA GATCCCAAAA ATTACCCAAA GAAGAAGATG |
| 721 | GAAAAGCGAT TTGTCTTCAA CAAGATAGAA ATCAATAACA |
| 761 | AGCTGGAATT TGAGTCTGCC CAGTTCCCCA ACTGGTACAT |
| 801 | CAGCACCTCT CAAGCAGAAA ACATGCCCGT CTTCCTGGGA |
| 841 | GGGACCAAAG GCGGCCAGGA TATAACTGAC TTCACCATGC |
| 881 | AATTTGTGTC TTCCTAAAGA GAGCTGTACC CAGAGAGTCC |
| 921 | TGTGCTGAAT GTGGACTCAA TCCCTAGGGC TGGCAGAAAG |
| 961 | GGAACAGAAA GGTTTTTGAG TACGGCTATA GCCTGGACTT |
| 1001 | TCCTGTTGTC TACACCAATG CCCAACTGCC TGCCTTAGGG |
| 1041 | TAGTGCTAAG AGGATCTCCT GTCCATCAGC CAGGACAGTC |
| 1081 | AGCTCTCTCC TTTCAGGGCC AATCCCCAGC CCTTTTGTTG |
| 1121 | AGCCAGGCCT CTCTCACCTC TCCTACTCAC TTAAAGCCCG |
| 1162 | CCTGACAGAA ACCACGGCCA CATTTGGTTC TAAGAAACCC |
| 1201 | TCTGTCATTC GCTCCCACAT TCTGATGAGC AACCGCTTCC |
| 1241 | CTATTTATTT ATTTATTTGT TTGTTTGTTT TATTCATTGG |
| 1281 | TCTAATTTAT TCAAAGGGGG CAAGAAGTAG CAGTGTCTGT |
| 1321 | PAAAGAGCCT AGTTTTTAAT AGCTATGGAA TCAATTCAAT |
| 1361 | TTGGACTGGT GTGCTCTCTT TAAATCAAGT CCTTTAATTA |
| 1401 | AGACTGAAAA TATATAAGCT CAGATTATTT AAATGGGAAT |
| 1441 | ATTTATAAAT GAGCAAATAT CATACTGTTC AATGGTTCTG |
| 1481 | AAATAAACTT CACTGAAG |

In another embodiment, the protease can be activated by thrombopoietin. Thrombopoietin is a glycoprotein with at least two forms, with apparent molecular masses of 25 kDa and 31 kDa, with a common N-terminal amino acid sequence. See, Bartley, et al., Cell 77: 1117-1124 (1994). An amino acid sequence for human thrombopoietin is available in the NCBI database at accession number NP 000451, gi:4507493. See website at ncbi.nlm.nih.gov. This sequence for human thrombopoietin is provided below as SEQ ID NO:15.

| | |
|---|---|
| 1 | MELTELLLVV MLLLTARLTL SSPAPPACDL RVLSKLLRDS |
| 41 | HVLHSRLSQC PEVHPLPTPV LLPAVDFSLG EWKTQMEETK |
| 81 | AQDTLGAVTL LLEGVMAARG QLGPTCLSSL LGQLSGQVRL |
| 121 | LLGALQSLLG TQLPPQGRTT AHKDPNAIFL SFQHLLRGKV |
| 161 | RFLMLVGGST LCVRPAPPTT AVPSRTSLVL TLNELPNRTS |
| 201 | GLLETNFTAS ARTTGSGLLK WQQGFRAKIP GLLNQTSRSL |
| 241 | DQIPGYLNRI HELLNGTRGL FPGPSRRTLG APDISSGTSD |
| 281 | TGSLPPNLQP GYSPSPTHPP TGQYTLFPLP PTLPTPVVQL |
| 321 | HPLLPDPSAP TPTPTSPLLN TSYTHSQNLS QEG |

A nucleotide sequence for human thrombopoietin is available in the NCBI database at accession number E 11965, gi:22025586. See website at ncbi.nlm.nih.gov. This sequence for human thrombopoietin is provided below as SEQ ID NO:16.

| | |
|---|---|
| 1 | ATGGAGCTGA CTGAATTGCT CCTCGTGGTC ATGCTTCTCC |
| 41 | TAACTGCAAG GCTAACGCTG TCCAGCCCGG CTCCTCCTGC |
| 81 | TTGTGACCTC CGAGTCCTCA GTAAACTGCT TCGTGACTCC |
| 121 | CATGTCCTTC ACAGCAGACT GAGCCAGTGC CCAGAGGTTC |
| 161 | ACCCTTTGCC TACACCTGTC CTGCTGCCTG CTGTGGACTT |
| 201 | TAGCTTGGGA GAATGGAAAA CCCAGATGGA GGAGACCAAG |
| 241 | GCACAGGACA TTCTGGGAGC AGTGACCCTT CTGCTGGAGG |
| 281 | GAGTGATGGC AGCACGGGGA CAACTGGGAC CCACTTGCCT |
| 321 | CTCATCCCTC CTGGGGCAGC TTTCTGGACA GGTCCGTCTC |
| 361 | CTCCTTGGGG CCCTGCAGAG CCTCCTTGGA ACCCAGCTTC |
| 401 | CTCCACAGGG CAGGACCACA GCTCACAAGG ATCCCAATGC |
| 441 | CATCTTCCTG AGCTTCCAAC ACCTGCTCCG AGGAAAGGTG |
| 481 | CGTTTCCTGA TGCTTGTAGG AGGGTCCACC CTCTGCGTCA |
| 521 | GGCGGGCCCC ACCCACCACA GCTGTCCCCA GCTGA |

In another embodiment, the protease can be activated by granulocyte colony stimulating factor (G-CSF). Granulocyte colony-stimulating factor is a glycoprotein secreted by macrophages, fibroblasts, and endothelial cells originally identified by its ability to stimulate the survival, proliferation, and differentiation in vitro of predominantly neutrophilic granulocytes from bone marrow progenitors. Nicola, N. A., Annu. Rev. Biochem. (1989) 58:45. An amino acid sequence for human G-CSF is available in the NCBI database at accession number CAA27290, gi:732764. See website at ncbi.nlm.nih.gov. This sequence for human G-CSF is provided below as SEQ ID NO:17.

| | |
|---|---|
| 1 | MAGPATQSPM KLMALQLLLW HSALWTVQEA TPLGPASSLP |
| 41 | QSFLLKCLEQ VRKIQGDGAA LQEKLCATYK LCHPEELVLL |
| 81 | GHSLGIPWAP LSSCPSQALQ LAGCLSQLHS GLFLYQGLLQ |
| 121 | ALEGISPELG PTLDTLQLDV ADFATTIWQQ MEELGMAPAL |
| 161 | QPTQGANPAF ASAFQRRAGG VLVASHLQSF LEVSYRVLRH |
| 201 | LAQP |

A nucleotide sequence for human G-CSF is available in the NCBI database at accession number X03655, gi:31693. See website at ncbi.nlm.nih.gov. This sequence for human G-CSF is provided below as SEQ ID NO:18.

| | |
|---|---|
| 1 | GGAGCCTGCA GCCCAGCCCC ACCCAGACCC ATGGCTGGAC |
| 41 | CTGCCACCCA GAGCCCCATG AAGCTGATGG CCCTGCAGCT |
| 81 | GCTGCTGTGG CACAGTGCAC TCTGGACAGT GCAGGAAGCC |
| 121 | ACCCCCCTGG GCCCTGCCAG CTCCCTGCCC CAGAGCTTCC |
| 161 | TGCTCAAGTG CTTAGAGCAA GTGAGGAAGA TCCAGGGCGA |
| 201 | TGGCGCAGCG CTCCAGGAGA AGCTGTGTGC CACCTACAAG |
| 241 | CTGTGCCACC CCGAGGAGCT GGTGCTGCTC GGACACTCTC |
| 281 | TGGGCATCCC CTGGGCTCCC CTGAGCAGCT GCCCCAGCCA |
| 321 | GGCCCTGCAG CTGGCAGGCT GCTTGAGCCA ACTCCATAGC |
| 361 | GGCCTTTTCC TCTACCAGGG GCTCCTGCAG GCCCTGGAAG |
| 401 | GGATCTCCCC CGAGTTGGGT CCCACCTTGG ACACACTGCA |
| 441 | GCTGGACGTC GCCGACTTTG CCACCACCAT CTGGCAGCAG |
| 481 | ATGGAAGAAC TGGGAATGGC CCCTGCCCTG CAGCCCACCC |
| 521 | AGGGTGCCAT GCCGGCCTTC GCCTCTGCTT TCCAGCGCCG |
| 561 | GGCAGGAGGG GTCCTAGTTG CCTCCCATCT GCAGAGCTTC |
| 601 | CTGGAGGTGT CGTACCGCGT TCTACGCCAC CTTGCCCAGC |
| 641 | CCTGAGCCAA GCCCTCCCCA TCCCATGTAT TTATCTCTAT |
| 681 | TTAATATTTA TGTCTATTTA AGCCTCATAT TTAAAGACAG |
| 721 | GGAAGAGCAG AACGGAGCCC CAGGCCTCTG TGTCCTTCCC |
| 761 | TGCATTTCTG AGTTTCATTC TCCTGCCTGT AGCAGTGAGA |
| 801 | AAAAGCTCCT GTCCTCCCAT CCCCTGGACT GGGAGGTAGA |
| 841 | TAGGTAAATA CCAAGTATTT ATTACTATGA CTGCTCCCCA |
| 881 | GCCCTGGCTC TGCAATGGGC ACTGGGATGA GCCGCTGTGA |
| 921 | GCCCCTGGTC CTGAGGGTCC CCACCTGGGA CCCTTGAGAG |
| 961 | TATCAGGTCT CCCACGTGGG AGACAAGAAA TCCCTGTTTA |
| 1001 | ATATTTAAAC AGCAGTGTTC CCCATCTGGG TCCTTGCACC |
| 1041 | CCTCACTCTG GCCTCAGCCG ACTGCACAGC GGCCCCTGCA |
| 1081 | TCCCCTTGGC TGTGAGGCCC CTGGACAAGC AGAGGTGGCC |
| 1121 | AGAGCTGGGA GGCATGGCCC TGGGGTCCCA CGAATTTGCT |
| 1161 | GGGGAATCTC GTTTTTCTTC TTAAGACTTT TGGGACATGG |
| 1201 | TTTGACTCCC GAACATCACC GACGCGTCTC CTGTTTTTCT |
| 1241 | GGGTGGCCTC GGGACACCTG CCCTGCCCCC ACGAGGGTCA |
| 1281 | GGACTGTGAC TCTTTTTAGG GCCAGGCAGG TGCCTGGACA |
| 1321 | TTTGCCTTGC TGGACGGGGA CTGGGGATGT GGGAGGGAGC |
| 1361 | AGACAGGAGO AATCATGTCA GGCCTGTGTG TGAAAGGAAG |
| 1401 | CTCCACTGTC ACCCTCCACC TCTTCACCCC CCACTCACCA |
| 1441 | GTGTCCCCTC CACTGTCACA TTGTAACTGA ACTTCAGGAT |
| 1481 | AATAAAGTGC TTGCCTCC |

An amino acid sequence for human G-CSF-2 is available in the NCBI database at accession number NP 000749, gi:27437030. See website at ncbi.nlm.nih.gov. This sequence for human G-CSF-2 is provided below as SEQ ID NO:19.

| | |
|---|---|
| 1 | MWLQSLLLLG TVACSTSAPA RSPSPSTQPW EHVNATQEAR |
| 41 | RLLNLSRDTA AEMNETVEVI SEMFDLQEPT CLQTRLELYK |
| 81 | QGLRGSLTKL KGPLTMMASH YKQHCPPTPE TSCATQTITF |
| 121 | ESFKENLKDF LLVTPFDCWE PVQE |

A nucleotide sequence for human G-CSF-2 is available in the NCBI database at accession number NM 000758, gi:27437029. See website at ncbi.nlm.nih.gov. This sequence for human G-CSF-2 is provided below as SEQ ID NO:20.

| | |
|---|---|
| 1 | ACACAGAGAG AAAGGCTAAA GTTCTCTGGA GGATGTGGCT |
| 41 | GCAGAGCCTG CTGCTCTTGG GCACTGTGGC CTGCAGCATC |
| 81 | TCTGCACCCG CCCGCTCGCC CAGCCCCAGC ACGCAGCCCT |
| 121 | GGGAGCATGT GAATGCCATC CAGGAGGCCC GGCGTCTCCT |
| 161 | GAACCTGAGT AGAGACACTG CTGCTGAGAT GAATGAAACA |
| 201 | GTAGAAGTCA TCTCAGAAAT GTTTGACCTC CAGGAGCCGA |
| 241 | CCTGCCTACA GACCCGCCTG GAGCTGTACA AGCAGGGCCT |
| 281 | GCGGGGCAGC CTCACCAAGC TCAAGGGCCC CTTGACCATG |
| 321 | ATGGCCAGCC ACTACAAGCA GCACTGCCCT CCAACCCCGG |
| 361 | AAACTTCCTG TGCAACCCAG ATTATCACCT TTGAAAGTTT |
| 401 | CAAAGAGAAC CTGAAGGACT TTCTGCTTGT CATCCCCTTT |
| 441 | GACTGCTGGG AGCCAGTCCA GGAGTGAGAC CGGCCAGATG |
| 481 | AGGCTGGCCA AGCCGGGGAG CTGCTCTCTC ATGAAACAAG |
| 521 | AGCTAGAAAC TCAGGATGGT CATCTTGGAG GGACCAAGGG |
| 561 | GTGGGCCACA GCCATGGTGG GAGTGGCCTG GACCTGCCCT |
| 601 | GGGCCACACT GACCCTGATA CAGGCATGGC AGAAGAATGG |
| 641 | GAATATTTTA TACTGACAGA AATCAGTAAT ATTTATATAT |
| 681 | TTATATTTTT AAAATATTTA TTTATTTATT TATTTAAGTT |
| 721 | CATATTCCAT ATTTATTCAA GATGTTTTAC CGTAATAATT |
| 761 | ATTATTAAAA ATATGCTTCT a |

An amino acid sequence for human G-CSF-3 is available in the NCBI database at accession number NP 757374, gi:27437051. See website at ncbi.nlm.nih.gov. This sequence for human G-CSF-3 is provided below as SEQ ID NO:21.

| | |
|---|---|
| 1 | MSPEPALSPA LQLLLWHSAL WTVQEATPLG PASSLPQSFL |
| 41 | LKCLEQVRKT QGDGAALQEK LCATYKLCHP EELVLLGHSL |
| 81 | GIPWAPLSSC PSQALQLAGC LSQLHSGLFL YQGLLQALEG |
| 121 | ISPELGPTLD TLQLDVADFA TTIWQQMEEL GMAPALQPTQ |
| 161 | GAMPAFASAF QRPAGGVLVA SHLQSFLEVS YRVLRHLAQP |

A nucleotide sequence for human G-CSF-3 is available in the NCBI database at accession number E 11965, gi:22025586. See website at ncbi.nlm.nih.gov. This sequence for human G-CSF-3 is provided below as SEQ ID NO:22.

| | |
|---|---|
| 1 | AAAACAGCCC GGAGCCTGCA GCCCAGCCCC ACCCAGACCC |
| 41 | ATGGCTGGAC CTGCCACCCA GAGCCCCATG AAGCTGATGG |
| 81 | GTGAGTGTCT TGGCCCAGGA TGGGAGAGCC GCCTGCCCTG |
| 121 | GCATGGGAGG GAGGCTGGTG TGACAGAGGG GCTGGGGATC |
| 161 | CCCGTTCTGG GAATGGGGAT TAAAGGCACC CAGTGTCCCC |
| 201 | GAGAGGGCCT CAGGTGGTAG GGAACAGCAT GTCTCCTGAG |
| 241 | CCCGCTCTGT CCCCAGCCCT GCAGCTGCTG CTGTGGCACA |
| 281 | GTGCACTCTG GACAGTGCAG GAAGCCACCC CCCTGGGCCC |
| 321 | TGCCAGCTCC CTGCCCCAGA GCTTCCTGCT CAAGTGCTTA |
| 361 | GAGCAAGTGA GGAAGATCCA GGGCGATGGC GCAGCGCTCC |
| 401 | AGGAGAAGCT GTGTGCCACC TACAAGCTGT GCCACCCCGA |
| 441 | GGAGCTGGTG CTGCTCGGAC ACTCTCTGGG CATCCCCTGG |
| 481 | GCTCCCCTGA GCAGCTGCCC CAGCCAGGCC CTGCAGCTGG |
| 521 | CAGGCTGCTT GAGCCAACTC CATAGCGGCC TTTTCCTCTA |
| 561 | CCAGGGGCTC CTGCAGGCCC TGGAAGGGAT CTCCCCCGAG |
| 601 | TTGGGTCCCA CCTTGGACAC ACTGCAGCTG GACGTCGCCG |
| 641 | ACTTTGCCAC CACCATCTGG CAGCAGATGG AAGAACTGGG |
| 681 | AATGGCCCCT GCCCTGCAGC CCACCCAGGG TGCCATGCCG |
| 721 | GCCTTCGCCT CTGCTTTCCA GCGCCGGGCA GGAGGGGTCC |
| 761 | TGGTTGCCTC CCATCTGCAG AGCTTCCTGG AGGTGTCGTA |
| 801 | CCGCGTTCTA CGCCACCTTG CCCAGCCCTG AGCCAAGCCC |
| 841 | TCCCCATCCC ATGTATTTAT CTCTATTTAA TATTTATGTC |
| 881 | TATTTAAGCC TCATATTTAA AGACAGGGAA GAGCAGAACG |
| 921 | GAGCCCCAGG CCTCTGTGTC CTTCCCTGCA TTTCTGAGTT |
| 961 | TCATTCTCCT GCCTGTAGCA GTGAGAAAAA GCTCCTGTCC |
| 1001 | TCCCATCCCC TGGACTGGGA GGTAGATAGG TAAATACCAA |
| 1041 | GTATTTATTA CTATGACTGC TCCCCAGCCC TGGCTCTGCA |
| 1081 | ATGGGCACTG GGATGAGCCG CTGTGAGCCC CTGGTCCTGA |
| 1121 | GGGTCCCCAC CTGGGACCCT TGAGAGTATC AGGTCTCCCA |
| 1161 | CGTGGGAGAC AAGAAATCCC TGTTTAATAT TTAAACAGCA |
| 1201 | GTGTTCCCCA TCTGGGTCCT TGCACCCCTC ACTCTGGCCT |
| 1241 | CAGCCGACTG CACAGCGGCC CCTGCATCCC CTTGGCTGTG |
| 1281 | AGGCCCCTGG ACAAGCAGAG GTGGCCAGAG CTGGGAGGCA |
| 1321 | TGGCCCTGGG GTCCCACGAA TTTGCTGGGG AATCTCGTTT |
| 1361 | TTCTTCTTAA GACTTTTGGG ACATGGTTTG ACTCCCGAAC |
| 1401 | ATCACCGACG TGTCTCCTGT TTTTCTGGGT GGCCTCGGGA |
| 1441 | CACCTGCCCT GCCCCCACGA GGGTCAGGAC TGTGACTCTT |
| 1481 | TTTAGGGCCA GGCAGGTGCC TGGACATTTG CCTTGCTGGA |
| 1521 | CGGGGACTGG GGATGTGGGA GGGAGCAGAC AGGAGGAATC |
| 1561 | ATGTCAGGCC TGTGTGTGAA AGGAAGCTCC ACTGTCACCC |
| 1601 | TCCACCTCTT CACCCCCCAC TCACCAGTGT CCCCTCCACT |
| 1641 | GTCACATTGT AACTGAACTT CAGGATAATA AAGTGTTTGC |
| 1681 | CTCCAAAAAA AAAAAAAAAA AAA |

In another embodiment, the protease can be activated by granulocyte macrophage colony stimulating factor (GM-CSF). Granulocyte macrophage colony-stimulating factor is a hematopoietic growth factor that promotes the proliferation and differentiation of hematopoietic progenitor cells. The cloned gene for GM-CSF has been expressed in bacteria, yeast and mammalian cells. The endogenous human protein is a monomeric glycoprotein with a molecular weight of about 22,000 daltons. GM-CSF produced in a yeast expression system is commercially available as Leukine™ from Immunex Corporation, Seattle, Wash. It is a glycoprotein of 127 amino acids characterized by three primary molecular species having molecular masses of 19,500, 16,800, and 15,500 daltons. An amino acid sequence for human GM-CSF is available in the NCBI database at accession number AAM44054, gi:21-218372. See website at ncbi.nlm.nih.gov. This sequence for human GM-CSF is provided below as SEQ ID NO:23.

| | |
|---|---|
| 1 | APARSPSPST QPWEHVNAIQ EARRLLNLSR DTAAEMNETV |
| 41 | EVISEMFDLQ EPTCLQTRLE LYKQGLRGSL TKLKGPLTKM |
| 81 | ASHYKQHCPP TPETSCATQT ITFESLKENL KDFLLVIPFD |
| 121 | CWEPVQE |

A nucleotide sequence for human GM-CSF is available in the NCBI database at accession number AF510855, gi:21218371. See website at ncbi.nlm.nih.gov. This sequence for human GM-CSF is provided below as SEQ ID NO:24.

| | |
|---|---|
| 1 | GCACCCGCCC GCTCGCCCAG CCCCAGCACG CAGCCCTGGG |
| 41 | AGCATGTGAA TGCCATCCAG GAGGCCCGGC GTCTCCTGAA |
| 81 | CCTGAGTAGA GACACTGCTG CTGAGATGAA TGAAACAGTA |
| 121 | GAAGTCATCT CAGAAATGTT TGACCTCCAG GAGCCGACCT |
| 161 | GCCTACAGAC CCGCCTGGAG CTGTACAAGC AGGGCCTGCG |
| 201 | GGGCAGCCTC ACCAAGCTCA AGGGCCCCTT GACCAAGATG |
| 241 | GCCAGCCACT ACAAGCAGCA CTGCCCTCCA ACCCCGGAAA |
| 281 | CTTCCTGTGC AACCCAGACT ATCACCTTTG AAAGTCTCAA |
| 321 | AGAGAACCTG AAGGACTTTC TGCTTGTCAT CCCCTTTGAC |
| 361 | TGCTGGGAGC CAGTCCAGGA GTGA |

In another embodiment, the protease can be activated by stromal cell-derived factor 1 (sdf-1). SDF-1 is a member of the chemokine family of pro-inflammatory mediators and is a potent chemoattractant for T cells, monocytes and lympho-hemopoietic progenitor cells. The expression of most chemokines is induced by cytokines, but SDF-1 is produced constitutively. Shirozu et al., (1995) Genomics, 28, 495-500. An amino acid sequence for human SDF-1 is available in the NCBI database at accession number P48061, gi:1352728. See website at ncbi.nlm.nih.gov. This sequence for human SDF-1 is provided below as SEQ ID NO:25.

| | |
|---|---|
| 1 | MNAKVVVVLV LVLTALCLSD GKPVSLSYRC PCRFFESHVA |
| 41 | RANVKHLKIL NTPNCALQIV ARLKNNNRQV CIDPKLKWIQ |
| 81 | EYLEKALNKR FKM |

A nucleotide sequence for human SDF-1 is available in the NCBI database at accession number NM 000609, gi:21218371. See website at ncbi.nlm.nih.gov. This sequence for human SDF-1 is provided below as SEQ ID NO:26.

| | |
|---|---|
| 1 | TCTCCGTCAG CCGCATTGCC CGCTCGGCGT CCGGCCCCCG |
| 41 | ACCCGTGCTC GTCCGCCCGC CCGCCCGCCC GCCCGCGCCA |
| 81 | TGAACGCCAA GGTCGTGGTC GTGCTGGTCC TCGTGCTGAC |
| 121 | CGCGCTCTGC CTCAGCGACG GGAAGCCCGT CAGCCTGAGC |
| 161 | TACAGATGCC CATGCCGATT CTTCGAAAGC CATGTTGCCA |
| 201 | GAGCCAACGT CAAGCATCTC AAAATTCTCA ACACTCCAAA |
| 241 | CTGTGCCCTT CAGATTGTAG CCCGGCTGAA GAACAACAAC |
| 281 | AGACAAGTGT GCATTGACCC GAAGCTAAAG TGGATTCAGG |
| 321 | AGTACCTGGA GAAAGCTTTA AACAAGAGGT TCAAGATGTG |
| 361 | AGAGGGTCAG ACGCCTGAGG AACCCTTACA GTAGGAGCCC |
| 401 | AGCTCTGAAA CCAGTGTTAG GGAAGGGCCT GCCACAGCCT |
| 441 | CCCCTGCCAG GCCAGGGCCC CAGGCATTGC CAAGGGCTTT |
| 481 | GTTTTGCACA CTTTGCCATA TTTTCACCAT TTGATTATGT |
| 521 | AGCAAAATAC ATGACATTTA TTTTTCATTT AGTTTGATTA |
| 561 | TTCAGTGTCA CTGGCGACAC GTAGCAGCTT AGACTAAGGC |
| 601 | CATTATTGTA CTTGCCTTAT TAGAGTGTCT TTCCACGGAG |
| 641 | CCACTCCTCT GACTCAGGGC TCCTGGGTTT TGTATTCTCT |
| 681 | GAGCTGTGCA GGTGGGGAGA CTGGGCTGAG GGAGCCTGGC |
| 721 | CCCATGGTCA GCCCTAGGGT GGAGAGCCAC CAAGAGGGAC |
| 761 | GCCTGGGGGT GCCAGGACCA GTCAACCTGG GCAAAGCCTA |
| 801 | GTGAAGGCTT CTCTCTGTGG GATGGGATGG TGGAGGGCCA |
| 841 | CATGGGAGGC TCACCCCCTT CTCCATCCAC ATGGGAGCCG |
| 881 | GGTCTGCCTC TTCTGGGAGG GCAGCAGGGC TACCCTGAGC |
| 921 | TGAGGCAGCA GTGTGAGGCC AGGGCAGAGT GAGACCCAGC |
| 961 | CCTCATCCCG AGCACCTCCA CATCCTCCAC GTTCTGCTCA |
| 1001 | TCATTCTCTG TCTCATCCAT CATCATGTGT GTCCACGACT |
| 1041 | GTCTCCATGG CCCCGCAAAA GGACTCTCAG GACCAAAGCT |
| 1081 | TTCATGTAAA CTGTGCACCA AGCAGGAAAT GAAAATGTCT |
| 1121 | TGTGTTACCT GAAAACACTG TGCACATCTG TGTCTTGTGT |
| 1161 | GGAATATTGT CCATTGTCCA ATCCTATGTT TTTGTTCAAA |
| 1201 | GCCAGCGTCC TCCTCTGTGA CCAATGTCTT GATGCATGCA |
| 1241 | CTGTTCCCCC TGTGCAGCCG CTGAGCGAGG AGATGCTCCT |
| 1281 | TGGGCCCTTT GAGTGCAGTC CTGATCAGAG CCGTGGTCCT |
| 1321 | TTGGGGTGAA CTACCTTGGT TCCCCCACTG ATCACAAAAA |
| 1361 | CATGGTGGGT CCATGGGCAG AGCCCAAGGG AATTCGGTGT |
| 1401 | GCACCAGGGT TGACCCCAGA GGATTGCTGC CCCATCAGTG |
| 1441 | CTCCCTCACA TGTCAGTACC TTCAAACTAG GGCCAAGCCC |
| 1481 | AGCACTGCTT GAGGAAAACA AGCATTCACA ACTTGTTTTT |
| 1521 | GGTTTTTAAA ACCCAGTCCA CAAAATAACC AATCCTGGAC |
| 1561 | ATGAAGATTC TTTCCCAATT CACATCTAAC CTCATCTTCT |
| 1601 | TCACCATTTG GCAATGCCAT CATCTCCTGC CTTCCTCCTG |
| 1641 | GGCCCTCTCT GCTCTGCGTG TCACCTGTGC TTCGGGCCCT |
| 1681 | TCCCACAGGA CATTTCTCTA AGAGAACAAT GTGCTATGTG |
| 1721 | AAGAGTAAGT CAACCTGCCT GACATTTGGA GTGTTCCCCT |
| 1761 | CCCACTGAGG GCAGTCGATA GAGCTGTATT AAGCCACTTA |
| 1801 | AAATGTTCAC TTTTGACAAA GGCAAGCACT TGTGGGTTTT |
| 1841 | TGTTTTGTTT TTCATTCAGT CTTACGAATA CTTTTGCCCT |
| 1881 | TTGATTAAAG ACTCCAGTTA AAAAAAATTT TAATGAAGAA |
| 1921 | AGTGGAAAAC AAGGAAGTCA AAGCAAGGAA ACTATGTAAC |
| 1961 | ATGTAGGAAG TAGGAAGTAA ATTATAGTGA TGTAATCTTG |
| 2001 | AATTGTAACT GTTCGTGAAT TTAATAATCT GTAGGGTAAT |
| 2041 | TAGTAACATG TGTTAAGTAT TTTCATAAGT ATTTCAAATT |
| 2081 | GGAGCTTCAT GGCAGAAGGC AAACCCATCA ACAAAAATTG |
| 2121 | TCCCTTAAAC AAAAATTAAA ATCCTCAATC CAGCTATGTT |
| 2161 | ATATTGAAAA AATAGAGCCT GAGGGATCTT TACTAGTTAT |
| 2201 | AAAGATACAG AACTCTTTCA AAACCTTTTG AAATTAACCT |
| 2241 | CTCACTATAC CAGTATAATT GAGTTTTCAG TGGGGCAGTC |
| 2281 | ATTATCCAGG TAATCCAAGA TATTTTAAAA TCTGTCACGT |
| 2321 | AGAACTTGGA TGTACCTGCC CCCAATCCAT GAACCAAGAC |
| 2361 | CATTGAATTC TTGGTTGAGG AAACAAACAT GACCCTAAAT |
| 2401 | CTTGACTACA GTCAGGAAAG GAATCATTTC TATTTCTCCT |
| 2441 | CCATGGGAGA AAATAGATAA GAGTAGAAAC TGCAGGGAAA |
| 2481 | ATTATTTGCA TAACAATTCC TCTACTAACA ATCAGCTCCT |
| 2521 | TCCTGGAGAC TGCCCAGCTA AAGCAATATG CATTTAAATA |
| 2561 | CAGTCTTCCA TTTGCAAGGG AAAAGTCTCT TGTAATCCGA |
| 2601 | ATCTCTTTTT GCTTTCGAAC TGCTAGTCAA GTGCGTCCAC |
| 2641 | GAGCTGTTTA CTAGGGATCC CTCATCTGTC CCTCCGGGAC |
| 2681 | CTGGTGCTGC CTCTACCTGA CACTCCCTTG GGCTCCCTGT |
| 2721 | AACCTCTTCA GAGGCCCTCG CTGCCAGCTC TGTATCAGGA |
| 2761 | CCCAGAGGAA GGGGCCAGAG GCTCGTTGAC TGCCTGTGTG |
| 2801 | TTGGGATTGA GTCTGTGCCA CGTGTATGTG CTGTGGTGTG |
| 2841 | TCCCCCTCTG TCCAGGCACT GAGATACCAG CGAGGAGGCT |
| 2881 | CCAGAGGGCA CTCTGCTTGT TATTAGAGAT TACCTCCTGA |
| 2921 | GAAAAAAGCT TCCGCTTGGA GCAGAGGGGC TGAATAGCAG |
| 2961 | AAGGTTGCAC CTCCCCCAAC CTTAGATGTT CTAAGTCTTT |
| 3001 | CCATTGGATC TCATTGGACC CTTCCATGGT GTGATCGTCT |
| 3041 | GACTGGTGTT ATCACCGTGG GCTCCCTGAC TGGGAGTTGA |
| 3081 | TCGCCTTTCC CAGGTGCTAC ACCCTTTTCC AGCTGGATGA |
| 3121 | GAATTTGAGT GCTCTGATCC CTCTACAGAG CTTCCCTGAC |
| 3161 | TCATTCTGAA GGAGCCCCAT TCCTGGGAAA TATTCCCTAG |
| 3201 | AAACTTCCAA ATCCCCTAAG CAGACCACTG ATAAAACCAT |
| 3241 | GTAGAAAATT TGTTATTTTG CAACCTCGCT GGACTCTCAG |
| 3281 | TCTCTGAGCA GTGAATGATT CAGTGTTAAA TGTGATGAAT |
| 3321 | ACTGTATTTT GTATTGTTTC AAGTGCATCT CCCAGATAAT |
| 3361 | GTGAAAATGG TCCAGGAGAA GGCCAATTCC TATACGCAGC |
| 3401 | GTGCTTTAAA AAATAPATAA GAAACAACTC TTTGAGAAAC |
| 3441 | AACAATTTCT ACTTTGAAGT CATACCAATG AAAAAATGTA |
| 3481 | TATGCACTTA TAATTTTCCT AATAAAGTTC TGTACTCAAA |
| 3521 | TGTA |

In another embodiment, the protease can be activated by fibroblast growth factor 4 (fgf-4). Fibroblast growth factors are a family of proteins involved in growth and differentiation in a wide range of contexts. These growth factors cause dimerization of their tyrosine kinase receptors leading to intracellular signaling. An amino acid sequence for human FGF-4 is available in the NCBI database at accession number NP 001998, gi:4503701. See website at ncbi.nlm.nih.gov. This sequence for human FGF-4 is provided below as SEQ ID NO:27.

| | |
|---|---|
| 1 | MSGPGTAAVA LLPAVLLALL APWAGRGGAA APTAPNGTLE |
| 41 | AELERRWESL VALSLARLPV AAQPKEAAVQ SGAGDYLLGI |
| 81 | KRLRRLYCNV GIGFHLQALP DGRIGGAHAD TRDSLLELSP |
| 121 | VERGVVSTFG VASRFFVAMS SKGKLYGSPF FTDECTFKET |
| 161 | LLPNNYNAYE SYKYPGMFIA LSKNGKTKKG NRVSPTMKVT |
| 201 | HFLPRL |

A nucleotide sequence for human FGF-4 is available in the NCBI database at accession number NM 002007, gi:4503700. See website at ncbi.nlm.nih.gov. This sequence for human FGF-4 is provided below as SEQ ID NO:28.

| | |
|---|---|
| 1 | GGGAGCGGGC GAGTAGGAGG GGGCGCCGGG CTATATATAT |
| 41 | AGCGGCCTCG GCCTCGGGCG GGCCTGGCGC TCAGGGAGGC |
| 81 | GCGCACTGCT CCTCAGAGTC CCAGCTCCAG CCGCGCGCTT |
| 121 | TCCGCCCGGC TCGCCGCTCC ATGCAGCCGG GGTAGAGCCC |
| 161 | GGCGCCCGGG GGCCCCGTCG CTTGCCTCCC GCACCTCCTC |
| 201 | GGTTGCGCAC TCCCGCCCGA GGTCGGCCGT GCGCTCCCGC |
| 241 | GGGACGCCAC AGGCGCAGCT CTGCCCCCCA GCTTCCCGGG |
| 281 | CGCACTGACC GCCTGACCGA CGCACGCCCT CGGGCCGGGA |
| 321 | TGTCGGGGCC CGGGACGGCC GCGGTAGCGC TGCTCCCGGC |
| 361 | GGTCCTGCTG GCCTTGCTGG CGCCCTGGGC GGGCCGAGGG |
| 401 | GGCGCCGCCG CACCCACTGC ACCCAACGGC ACGCTGGAGG |
| 441 | CCGAGCTGGA GCGCCGCTGG GAGAGCCTCG TGGCGCTCTC |
| 481 | GTTGGCGCGC CTGCCGGTGG CAGCGCAGCC CAAGGAGGCG |
| 521 | GCCGTCCAGA GCGGCGCCGG CGACTACCTG CTGGGCATCA |
| 561 | AGCGGCTGCG GCGGCTCTAC TGCAACGTGG GCATCGGCTT |
| 601 | CCACCTCCAG GCGCTCCCCG ACGGCCGCAT CGGCGGCGCG |
| 641 | CACGCGGACA CCCGCGACAG CCTGCTGGAG CTCTCGCCCG |
| 681 | TGGAGCGGGG CGTGGTGAGC ATCTTCGGCG TGGCCAGCCG |
| 721 | GTTCTTCGTG GCCATGAGCA GCAAGGGCAA GCTCTATGGC |
| 761 | TCGCCCTTCT TCACCGATGA GTGCACGTTC AAGGAGATTC |
| 801 | TCCTTCCCAA CAACTACAAC GCCTACGAGT CCTACAAGTA |
| 841 | CCCCGGCATG TTCATCGCCC TGAGCAAGAA TGGGAAGACC |
| 881 | AAGAAGGGGA ACCGAGTGTC GCCCACCATG AAGGTCACCC |
| 921 | ACTTCCTCCC CAGGCTGTGA CCCTCCAGAG GACCCTTGCC |
| 961 | TCAGCCTCGG GAAGCCCCTG GGAGGGCAGT GCGAGGGTCA |
| 1001 | CCTTGGTGCA CTTTCTTCGG ATGAAGAGTT TAATGCAAGA |
| 1041 | GTAGGTGTAA GATATTTAAA TTAATTATTT AAATGTGTAT |
| 1081 | ATATTGCCAC CAAATTATTT ATAGTTCTGC GGGTGTGTTT |
| 1121 | TTTAATTTTC TGGGGGGAAA AAAAGACAAA ACAAAAAACC |
| 1161 | AACTCTGACT TTTCTGGTGC AACAGTGGAG AATCTTACCA |
| 1201 | TTGGATTTCT TTAACTTGT |

Stem Cell Recruitment

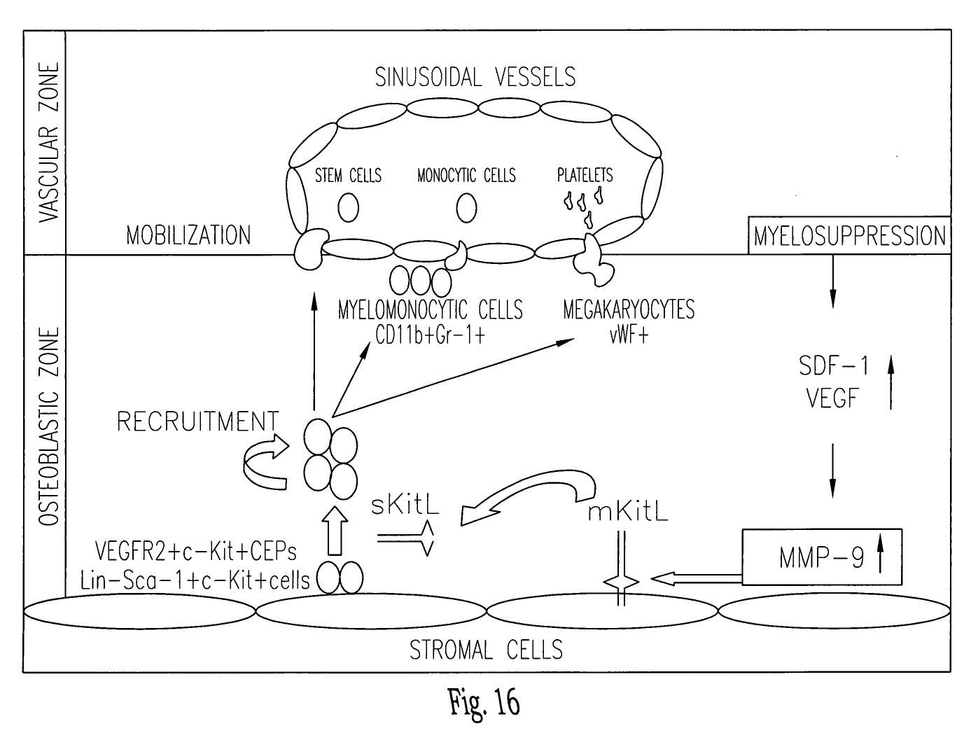

Rapid recruitment of hematopoietic stem cells from their quiescent niche is essential to repopulate progenitors of the myeloid and megakaryocytic lineage to avoid life-threatening infection and bleeding after bone marrow suppression. Physiological stress induces rapid recruitment of stem cells from their bone marrow niche with subsequent mobilization to the circulation, where they home to respective organs and either contribute to restore organ function or regenerate the stem and progenitor cell pool. According to the invention, matrix metalloproteinase 9 (MMP-9) and other proteases play a key role in the release of the stem cell-active cytokine sKitL, thereby directing stem and progenitor cell recruitment and expansion, and facilitating hematopoietic reconstitution (FIG. 16).

According to the invention, bone marrow suppression results in a timely up-regulation of MMP-9 within the bone marrow microenvironment with the release of sKitL. Increased bioactive sKitL promotes hematopoietic stem cell cycling and enhances their motility, which are essential for hematopoietic stem cell and progenitor survival and differentiation. MMP-9 activation also facilitates mobilization of bone marrow repopulating cells into the peripheral circulation, a process that may be essential for reconstitution of the stem cell pool. Collectively, the experimental results described herein indicate that activation of a protease (e.g. a metalloproteinase) serves as the decisive checkpoint for the rapid reconstitution of the hematopoiesis following life-threatening stressors.

For example, using the compositions, cells and methods provided herein a number of injuries, diseases and conditions can be treated, including hemoimmunological recovery through normalization of hemopoiesis in the recipient and contribution to the treatment of chronic myelocytic leukemia (CML), acute myelocytic leukemia (AML), acute lymphocytic leukemia (ALL), malignant lymphoma, multiple myeloma, aplastic anemia gravis, myelodysplastic syndrome (MDS) and other hereditary diseases, treatment of autoimmune diseases, and gene therapy by the gene transfer technique. Moreover, the compositions, cells and methods of the invention may reduce or replace the need for tissue transplants and thereby reduce graft failure or tissue rejection problems. In other embodiment, the compositions, cells and methods of the invention can be used in conjunction with tissue transplantation to facilitate healing and incorporation of the new tissue.

Hematopoietic stem cells and bone marrow-derived endothelial precursor cells reside in a microenvironment where they can readily sense and respond to the stress-induced demands for supporting hematopoiesis and angiogenesis. To meet such a high demand, rapid availability of cytokines is essential for the recruitment of quiescent hematopoietic stem cells and endothelial precursor cells to a permissive niche where they can proliferate, differentiate and replenish the exhausted progenitor and precursor pool. As provided herein, this microenvironmental switch depends on the up-regulation of MMP-9 by bone marrow cells, resulting in increased bioavailability of sKitL.

MMP-9 cleaves several cytokines and/or its receptors, processes that can either activate or inactivate the cytokines (Vu and Werb, 2000). For example, active MMP-9 may release membrane-bound KitL (mKitL). A precedent for such a process is the MMP-9 induced VEGF release from the tethered pool within the microenvironment of developing bone (Engsig et al., 2000) or within tumors of pancreatic islets (Bergers et al., 2000). mKitL is a glycoprotein of 248 amino acids that is rapidly cleaved from the cell to release an active soluble protein of 164 amino acids. In contrast, a glycoprotein of 220 amino acids, which lacks the proteolytic cleavage site encoded by differentially spliced exon 6 sequences remains predominantly membrane-associated (Huang et al., 1992). The only enzyme previously reported to cleave mKitL is a mast cell chymase (Longley et al., 1997). The extracellular domain of mKitL has two potential consensus sequences that can be hydrolyzed by MMP-9 (Kridel et al., 2001). Thus, according to the invention, the rapid increase in plasma sKitL levels in MMP-9^{+/+} mice, and the relative deficiency of sKitL at baseline or after myelosuppression in MMP-9^{-/-} mice, indicate that MMP-9 plays a physiological role in releasing sKitL setting up the stage for hematopoietic reconstitution.

Rapid hematopoietic recovery following chemotherapy depends on the ability of quiescent stem cells to enter cell cycle (Morrison et al., 1997a,b). KitL stands out as a key modulator of stem cell function affecting growth, survival and/or differentiation of hematopoietic cells and driving hematopoietic reconstitution after bone marrow ablation. Although MMP-9 null mice have cell-associated KitL, the low levels of sKitL, as seen in the MMP-9^{-/-} mice, results in delayed entry of stem cells into the S-phase of cell cycle leading to diminished cell motility and differentiation. Hence, MMP-9 can increase KitL levels during hematopoietic recovery after bone marrow suppression or during stem cell mobilization.

The biological significance of sKitL and mKitL in the regulation of post-natal hematopoiesis is not understood. Homozygous Steel Dickie S1^{d}/S1^{d} mice, which lack mKitL, have defective hematopoiesis under steady state conditions, suggesting that mKitL may play a role in survival of hematopoietic stem cells. However, since it is difficult to generate transgenic mice completely deficient in sKitL, the physiological significance of sKitL under steady state conditions has remained unknown (Tajima et al., 1998). However, as provided herein, while MMP-9-mediated release of sKitL may not be required for steady state hematopoiesis, MMP-9 is essential for the rapid hematopoietic reconstitution during the critical pancytopenic period when there is exhaustion of precursor/progenitor cells. Despite germ-free conditions where the 5-fluorouracil treated mice were kept in the studies described herein, the delay in hematopoietic recovery was translated into the demise of 72% of the 5-fluorouracil-treated MMP-9^{-/-} mice, while all of the 5-fluorouracil-treated MMP-9^{+/+} mice survived. Under non-sterile conditions significantly more of the 5-fluorouracil treated MMP-9^{-/-} mice would likely have succumbed to the complications of prolonged neutropenia and thrombocytopenia. These findings underscore the need for MMP-9 in addition to mKitL to restore hematopoiesis.

In humans, rapid bone marrow recovery after repetitive treatment with chemotherapeutic agents is essential to avoid morbidity and mortality secondary to prolonged bone marrow suppression. Indeed, life-threatening infections and bleeding are direct effects of prolonged chemotherapy-related neutropenia and thrombocytopenia. Chronic use of certain chemotherapeutic agents may alter MMP-9 secretion and activation and therefore, induce long-term bone marrow suppression or disrupt trafficking of hematopoietic stem cells contributing to bone marrow failure states. On the other hand, inhibition of MMP-9 may provide a novel mechanism to regulate hematopoiesis in myeloproliferative disorders.

Up-regulation of adhesion molecules and chemokine receptors on hematopoietic stem cells, progenitors and CEPs facilitate their recruitment within the bone marrow followed by mobilization to the circulation. The experimental results provided herein show that MMP-9 mediated release of sKitL enhances the mitogenic potential of stem and progenitor cells translocating them from their quiescent into a permissive proliferative vascular niche. sKitL is a potent cytokine that not only increases the motility of hematopoietic stem cells and progenitors within the bone marrow, but also sets up the stage for hematopoietic cells to be launched to the circulation (Long et al., 1992; Papayannopoulou et al., 1998).

After elimination of the cycling stem cell pool with 5-fluorouracil treatment, the inventors observed hematopoietic cell clusters in close contact to osteoblasts (osteoblastic zone) on day 3 in both wild type and MMP-9^{-/-} mice. However, at later time points, hematopoietic clusters in MMP-9^{-/-} mice did not repopulate the inner vascular zone. These results indicate that MMP-9-mediated release of sKitL increases the mitogenic potential of hematopoietic stem cells, shifting hematopoietic stem cells into a vascular enriched microenvironment where they receive instructions for differentiation and mobilization.

Increased MMP-9 has been detected in the serum of IL-8 treated monkeys and some link to the mobilization of progenitor cells has been suggested (Pruijt et al., 1999). These authors hypothesized that the 11-8-induced activation of neutrophils generates high levels of MMP-9. In another report, intravenous injection of recombinant MMP-9 into rabbits produced an rapid, transient neutropenia, followed by a profound neutrophilia and the appearance of immature myeloid cells, including blast cells (Masure et al., 1997). These results can be explained by MMP-9-mediated release of sKitL.

The inventors have previously worked on endothelial-active cytokines, such as VEGF, and SDF-1 and their role in inducing mobilization of bone marrow repopulating cells (Hattori et al., 2001a, b). According to the invention, MMPs are necessary intermediates downstream of these factors. Cytokine-induced mobilization of hematopoietic progenitors and cells with hematopoietic stem cell potential was markedly impaired in MPI-treated or MMP-9^{-/-} mice.

Mobilization of stem and progenitor cells seems to be essential for reconstitution of the stem cell pool in murine myelosuppression models. Indeed, mobilization and localization of bone marrow-derived stem cells to the spleen may facilitate the expansion of the stem/progenitor cell pools. In this regard, MMP-9 activation not only promotes trilineage hematopoietic recovery, but also facilitates mobilization of hematopoietic stem cells and endothelial precursor cells, a process essential for hematopoietic reconstitution and replenishment of stem and progenitor cells.

Bone marrow not only provides a suitable microenvironment for hematopoietic stem cells, but is also a dispensable reservoir for organ-specific stem cells for endothelium, muscle, brain, pancreas, and liver cells (Krause et al., 2001). c-Kit is expressed on hematopoietic stem cells and progenitor cells (Peichev et al. 2000) and cardiac precursors (Orlic et al., 2001). Selective recruitment of bone marrow-derived stem cells is critical for supporting post-natal organogenesis and tumorigenesis (Coussens et al., 2000; Lyden et al., 2001). The inventors have found that mobilization of both hematopoietic stem cells and VEGFR2⁺ progenitor cells to the circulation occurs in response to VEGF and is MMP-9 dependent (FIG. 16). These data suggest that MMP-9 activation is not only a decisive checkpoint for recruitment of hematopoietic stem cells but also other types of undifferentiated cells, such as endothelial progenitors.

Taken together, the data provided herein show that stem and progenitor cell recruitment from the quiescent niche into a permissive microenvironment following stress depends on MMP-9. This protease acts on different levels by directly releasing sKitL that affects cell mobility and by promoting recruitment of stem and progenitor cells, thereby enhancing rapid differentiation. Stem cell-stromal cell interactions are amplified by membrane-anchored cytokines, by transducing proliferation and/or differentiation signals, and by the maintenance of progenitors in a quiescent state under the constraints of limited growth factor concentrations. It is intriguing that the conversion of KitL from a membrane-bound, adhesion/survival-promoting molecule to a soluble survival/mitogenic factor by MMP-9 is a critical event in regulating the stem cell niche. Because modulating the bioavailability of local cytokines changes the hematopoietic stem cell fate, regulators of enzyme activity leading to proteolytic cleavage are critical elements in the determination of the hematopoietic stem cell fate. It will be interesting to determine if MMP-9 is essential for the recruitment of other stem cells that express c-Kit, and if there is an analogous specific protease-dependent regulatory process regulating the stem cell niche for c-Kit negative stem and progenitor cells

Embryonic stem cells provide a rich source of pluripotent stem cells. Derivation of organ-specific stem cells from embryonic stem cells provide unlimited sources of cells that can be used for therapeutic purposes. Stem cells giving rise to heart tissue can be used to repair the damaged heart muscle immediately after heart attack. Stem cells programmed to differentiate into blood cells can be used to restore blood production in patients who have failed to be successfully engrafted with transplanted bone marrow, or do not have appropriate match. However, given the bio-ethical and political issues involved in the obtaining and processing of human embryonic stem cells, the use of these cells have confronted with major obstacles. Moreover, embryonic stem cells, lacking the appropriate developmental instructions are less likely to have the potential to functionally engraft into an adult tissue.

An alternative source of stem cells is adult tissues such as bone marrow. Numerous studies have demonstrated that adult bone marrow contain rare population of organ-specific stem cells that can functionally contribute to organ-regeneration and blood vessel formation. However up to now the use of organ specific stem cells from adult bone marrow have been hampered because of the lack of frequency and the lack of knowledge of the mechanism that prompts these cells to be recruited from the safe haven of bone marrow environment and mobilized into the peripheral blood where they incorporate into damaged tissue.

The inventors have identified a novel mechanism by which this rare population of bone marrow derived stem cells is called upon to undergo expansion and mobilize to the peripheral circulation. The inventors have demonstrated that physiological stress results in the activation of metalloproteinase-9 (MMP-9) in the bone marrow cells. Activated MMP-9 promotes the release of an otherwise tethered stem cell active cytokine known as Kit-ligand. The enhanced bio-availability of this stem cell factor promotes the expansion and increases the mobility of hibernating sessile stem cells thereby translocating them to a permissive environment that is conducive to expansion and mobilization to the peripheral blood.

The inventors have demonstrated that even though MMP-9 deficient mice have no apparent defects, they failed to recover after receiving high doses of a chemotherapeutic agents. Compared to the control wild-type mice close to 70% of the mice deficient in MMP-9 died from complication of marrow suppression which included hemorrhage and bleeding. This finding suggested that activation of MMP-9 may be essential for reconstitution of blood stem cells. Further investigation demonstrated that MMP-9 promotes release of stem cell active cytokines, thereby promoting expansion of quiescent stem cells. This novel concept lays the foundation of developing strategies where activation of proteases such as MMP-9 may act as molecular switches to expand a large population of stem cells that may ultimately be used for organ-regeneration and tissue vascularization. As compared to embryonic derived stem cells, as adult derived stem cells are endowed with additional developmental instructions, they may be better suited for therapeutic purposes.

Genetic Manipulation

Delivery and introduction of the nucleic acids into the cells can be accomplished by various methods well known to the art worker, for example, using the methods disclosed in I. D. Goldfine, M. S. German, H. C. Tseng, J. Wang, J. L. Bolaffi, J. W. Chen, D. C. Olson and S. S. Rothman, The endocrine secretion of human insulin and growth hormone by exocrine glands of the gastrointestinal tract, Nat Biotechnol 15 (1997) 1378-82, and U.S. Pat. Nos. 5,837,693; 5,885,971; 6,004,944; 6,225,290; 6,255,289; or 6,258,789. For example, transformation of cells (e.g. stem or progenitor cells) can be accomplished by administering the DNA of interest directly to the mammalian subject (in vivo gene therapy), or into in vitro cultures of cells that are subsequently transplanted into the mammalian subject after transformation (ex vivo gene therapy).

The DNA of interest can be delivered to the subject or the in vitro cell culture as, for example, purified DNA, in a viral vector (e.g., adenovirus, mumps virus, retrovirus), a DNA- or RNA-liposome complex, or by utilizing cell-mediated gene transfer. Further, the vector, when present in non-viral form, may be administered as a DNA or RNA sequence-containing chemical formulation coupled to a carrier molecule which facilitates delivery to the host cell. Such carrier molecules can, for example, include an antibody specific to an antigen expressed on the surface of the targeted stem or progenitor cells, or some other molecule capable of interaction with a receptor associated with the cell type of interest.

The DNA or RNA sequence encoding the molecule used in accordance with the invention may be either locally or systemically administered to the mammalian subject, which may be a human or a non-human mammal (e.g., bovine, equine, canine, mouse, rat or feline). Where the targeted cells are bone marrow cells, local administration may be by injection into bone marrow or into the vascular system feeding the tissue of interest (e.g. the tissue in which the quiescent stem or progenitor cells exist). Systemic administration can be carried out by intramuscular injection of a viral vector containing the DNA of interest. Where the targeted tissue is bone marrow or liver, systemic administration may be by direct administration of a viral vector containing a DNA of interest, for example, an adenovirus vector, a mumps virus vector or other virus vector which infects cells of the bone marrow or liver. Alternatively, administration may be achieved by administration of the DNA of interest in a viral vector or DNA-containing formulation (e.g. liposome) which binds a marker or receptor for the stem cells or progenitor cells of interest.

As indicated above, the cells of a patient may be transformed ex vivo by collecting bone marrow cells (or other sources of stem or progenitor cell), culturing the collected cells in vitro, and transfecting the cultured with a DNA of interest in vitro. The resulting transformed cells are then implanted into the mammalian subject, for example, into the corresponding tissue of the mammalian subject from which the biopsy was taken.

The selected cells may be transformed in vivo by mechanical means (e.g., direct injection of the DNA of interest into or in the region of the tissue or lipofection) or by biological means (e.g., infection of a tissue with a non-pathogenic virus, for example, a non-replicative virus, containing the DNA of interest). For example, the selected host cells can be transformed in vivo by infection with a non-replicative virus containing the DNA of interest.

Preparation of the host cells for transformation will depend upon several factors such as whether transformation is performed ex vivo or in vivo, the tissue targeted for gene transfer, the route of administration, and whether a biological or non-biological vector is employed. For example, where the preparation for transformation is administered via the oral route, the preparation may be formulated to provide mucosal resistance (e.g., resistance to proteolytic digestion, denaturation in the mucosal environment, etc.). In addition to the DNA of interest, such oral preparations can include detergents, gelatins, capsules, or other delivery vehicles to protect against degradation.

Generally, transformation is accomplished either by infection of host cells with a virus, for example, with a replication-deficient virus, containing the DNA of interest, or by a non-viral transformation method, such as direct injection of the DNA into or near the target tissue or cells, lipofection, “gene gun”, or other methods well known in the art. The methodology is dependent upon whether the gene transfer is performed ex vivo or in vivo.

Ex vivo gene therapy is accomplished by obtaining cells from the tissue of interest and establishing a primary culture of these cells. Methods for obtaining tissue biopsies and growing cells from this tissue in vitro are available in the art. Methods for separation of cells from tissue (see, for example, Amsterdam et al., J. Cell Biol. 63:1057-1073, 1974), and methods for culturing cells in vitro are well known in the art.

The cells in the in vitro culture are then transformed using various methods known in the art. For example, transformation can be performed by calcium or strontium phosphate treatment, microinjection, electroporation, lipofection, or viral infection. For example, the cells may be injected with a moloney-LTR driven construct or lipoinfected with an adenovirus, vaccinia virus, HIV, or CMV promoter construct. The transfected DNA plasmid can contain a selectable marker gene or be co-transfected with a plasmid containing a selectable marker.

Where one or more selectable markers are transferred into the cells along with the DNA of interest, the cell populations containing the DNA of interest can be identified and enriched by selecting for the marker(s). Typically markers provide for resistance to antibiotics such as tetracycline, hygromycin, neomycin, and the like. Other markers can include thymidine kinase and the like.

The ability of the transformed cells to express the DNA of interest can be assessed by various methods known in the art. For example, the ability of the cells to secrete protein into the cell culture media can be examined by performing an ELISA on a sample of cell culture supernatant using an antibody which specifically binds the protein (e.g. a protease) encoded by the DNA of interest. Alternatively, expression of the DNA of interest can be examined by Northern blot to detect mRNA which hybridizes with a DNA probe derived a selected sequence of the DNA of interest. Those cells that contain the DNA of interest can be further isolated and expanded via in vitro culture using methods well known in the art.

After expansion of the transformed cells in vitro, the cells are implanted into the mammalian subject, for example, into the bone marrow from which the cells were originally derived, by methods available in the art. The cells can be implanted in an area of dense vascularization, and in a manner that minimizes evidence of surgery in the subject. The engraftment of the implant of transformed cells is monitored by examining the mammalian subject for classic signs of graft rejection, i.e., inflammation and/or exfoliation at the site of implantation, and fever.

In vivo transformation methods may employ either a biological means of introducing the DNA into the target cells (e.g., a virus containing the DNA of interest) or a mechanical means to introduce the DNA into the target cells (e.g., direct injection of DNA into the cells, liposome fusion, pneumatic injection using a “gene gun”). The biological means used for in vivo transformation of target cells may be a virus, particularly a virus which is capable of infecting the target cell, and integrating at least the DNA of interest into the target cell's genome, but is not capable of replicating. Such viruses are referred to as replication-deficient viruses or replication-deficient viral vectors. Alternatively, the virus containing the DNA of interest is attenuated, i.e. does not cause significant pathology or morbidity in the infected host (i.e., the virus is nonpathogenic or causes only minor disease symptoms).

Numerous viral vectors useful for in vivo transformation and gene therapy are known in the art, or can be readily constructed given the skill and knowledge in the art. Viruses include non-replicative mutants/variants of adenovirus, mumps virus, retrovirus, adeno-associated virus, herpes simplex virus (HSV), cytomegalovirus (CMV), vaccinia virus, and poliovirus. The replication-deficient virus may be capable of infecting replicating and/or undifferentiated cells, because stem and progenitor cells are primarily composed of these cell types. The viral vector may also be specific or substantially specific for cells of the targeted tissue.

In vivo gene transfer using a biological means can be accomplished by administering the virus containing the DNA to the mammalian subject either by an oral route, or by injection depending upon the tissue targeted for gene transfer. The amount of DNA and/or the number of infectious viral particles effective to infect the targeted tissue, transform a sufficient number of cells, and provide for expression of therapeutic levels of the protein can be readily determined based upon such factors as the efficiency of the transformation in vitro, the levels of expression achieved in vitro, and the susceptibility of the targeted cells to transformation. For example, where the targeted cells are stem cells and where a virus containing the DNA of interest is administered into the bone marrow, the virus will be administered at a concentration effective to infect bone marrow cells of the mammalian subject and provide for expression of the nucleic acid sequence at levels sufficient to recruit stem or progenitor cells from the bone marrow.

Various mechanical means can be used to introduce a DNA of interest directly into a bone marrow for expression in stem cells or progenitor cells of a mammalian subject. For example, the DNA of interest may be introduced into a bone marrow by injection. Alternatively, administration of the virus containing the DNA of interest may be accomplished by intramuscular injection.

The DNA of interest may be naked (i.e., not encapsulated), provided as a formulation of DNA and cationic compounds (e.g., dextran sulfate), or may be contained within liposomes. Alternatively, the DNA of interest can be pneumatically delivered using a “gene gun” and associated techniques which are well known in the art (Fynan et al. Proc. Natl. Acad. Sci. USA 90:11478-11482, 1993).

The amount of DNA administered will vary according to a number of factors including the susceptibility of the target cells to transformation, the size and weight of the subject, the levels of protein expression desired, and the condition to be treated. For example, the amount of DNA injected into bone marrow of a human may be from about 1 μg to 200 mg, from about 100 μg to 100 mg, from about 500 μg to 50 mg, or about 10 mg. Generally, the amounts of DNA for human gene therapy can be extrapolated from the amounts of DNA effective for gene therapy in an animal model. The amount of DNA necessary to accomplish stem or progenitor cell transformation will decrease with an increase in the efficiency of the transformation method used.

Following either ex vivo or in vivo transfer of a DNA of interest into the tissue of interest, the effects of expression of the DNA of interest can be monitored in a variety of ways. Generally, the presence of a secreted protein in either a sample of blood, or a sample of saliva, pancreatic juices, urine, or mucosal secretions from the subject can be assayed for the presence of the secreted protein. Appropriate assays for detecting a protein of interest in either saliva or blood samples are well known in the art. For example, where gene therapy has been performed to accomplish stem cell recruitment, a sample of blood can be tested for the presence of the protease or heightened levels of stem cells using an antibody which specifically binds the protease or the stem cells in an ELISA assay. This assay can be performed either qualitatively or quantitatively. The ELISA assay, as well as other immunological assays, are described in Antibodies: A Laboratory Manual (1988, Harlow and Lane, eds. Cold Spring Harbor Laboratory, Cold Spring Harbor, N.Y.).

Alternatively, or in addition, the efficacy of the therapy can be assessed by testing or observing the patient. For example, small blood samples may be taken and evaluated for hematopoietic cells and factors. For example, where the recombinant protein has protease activity, the efficacy of therapy can be tested monitoring the condition of the mammalian subject for improved hematopoiesis and for the absence of anemia.

According to the invention, any type of stem cell or progenitor cell can be genetically manipulated in order to promote stem cell recruitment. Pluripotent stem cells are capable of developing into more than two types of mature cells, such as endothelial cells, hematopoietic cells, and at least one other type of cell. Bipotent stem cells are capable of developing into two types of mature cells, such as endothelial cells and hematopoietic cells. Progenitor cells are capable of developing into one type of mature cells, such as endothelial cells or hematopoietic cells. Pluripotent stem cells, bipotent stem cells, and progenitor cells are capable of developing into mature cells either directly, or indirectly through one or more intermediate stem or progenitor cells. An endothelial stem cell is a stem cell that is capable of maturing into at least one type of mature endothelial cell. The stem cells provided herein may be pluripotent, bipotent, or monopotent. Monopotent stem cells are also referred to as progenitor cells

Stem cells, progenitor cells and precursor cells, for example that are used to generate transgenic cells, can be isolated directly from bone marrow, circulating peripheral blood, and autologous umbilical cord blood or from specific niches within each organ. The leukocyte fraction of peripheral blood is a useful source of precursor cells. In addition, precursor cells can be produced in vitro or in vivo through the differentiation of stem cells. For example, in addition to giving rise to cells such as B and T lymphocytes, granulocytes, and monocytes, hematopoietic stem cells isolated from adult human bone marrow also differentiate into non-hematopoietic lineages (lin⁻) that give rise to endothelial cell precursors (Otani et al., Nature Medicine, 2002, 8(9): 1004-1010).

Endothelial precursor cells can be identified by their surface antigens and/or by the factors they express. Such antigens include, for example, one or more vascular endothelial growth factor receptors (VEGFR). Examples of VEGF receptors include Flk-1 and Flt-1. The Flk-1 receptor is also known by other names, such as vascular endothelial growth factor receptor-2 (VEGFR-2), which is now the standard name for this receptor. Human Flk-1 is also referred to in the literature and herein as KDR. Bone-marrow reconstituting hematopoietic stem cells and endothelial cell precursors both have the CD34 antigenic determinant (U.S. Pat. No. 5,980,887) and express vascular endothelial growth factor receptor-1 (VEGFR-1). Endothelial progenitor cells also express the antigenic determinant AC133 (Peichev et al., Blood, 2000, 95(3):952-958). Endothelial progenitor cells and vascularizing endothelial cells also express VEGFR-2.

Hematopoictic stem cells, and other stem cells including cardiac, endothelial and epithelial cells, express c-Kit, the receptor for KitL. Mutations in KitL or c-Kit produce defects in germ cell and melanocyte development, impairment of hematopoiesis and increased sensitivity to radiation and chemotherapy. Hence, KitL may play a key role in maintaining and reconstituting the stem cell pool in adults

At least some endothelial progenitor cells also express the CD34+ marker. The endothelial progenitor cells may be further characterized by the absence or significantly lower expression levels of certain markers characteristic of mature cells. Such markers include CD1, CD3, CD8, CD10, CD13, CD14, CD15, CD19, CD20, CD33, and CD41A.

In addition, at least some endothelial progenitor cells also express the AC133 antigen, which was described by Yin et al. in Blood 90, 5002-5112 (1997) and by Miraglia et al. in Blood 90, 5013-5021 (1997). The AC133 antigen is expressed on endothelial and hematopoietic precursor cells, but not on mature cells.

Most, if not all, of the endothelial precursor cells express VEGFR-2 (Flk-1, KDR). The CD34 marker is characteristic of precursor cells, such as angioblasts and hematopoietic progenitor cells. Approximately 0.5-10% of CD34+ cells are also VEGFR-2+ and approximately 1% of bone marrow cells are CD34+. Of these CD34+ cells, approximately 1% are VEGFR-2+.

High levels of c-kit RNA transcripts are found in primary bone marrow derived mast cells and mast cell lines, while somewhat lower levels are found in melanocytes and erythroid cell lines. Hence c-kit expression is another marker for endothelial progenitor cells. The c-kit proto-oncogene encodes a transmembrane tyrosine kinase receptor for an unidentified ligand and is a member of the colony stimulating factor-1 (CSF-1)—platelet-derived growth factor (PDGF)—kit receptor subfamily (Besmer et al., (1986) Nature 320, 415-421; Qiu et al., (1988) EMBO J. 7, 1003-1011; Yarden et al., (1987) EMBO J. 6, 3341-3351; Majumder, S., Brown, K., Qiu, F.-H. and Besmer, P. (1988) Mol. Cell. Biol. 8, 4896-4903). c-kit is allelic with the white-spotting (W) locus of the mouse. Mutations at the W locus affect proliferation and/or migration and differentiation of germ cells, pigment cells and distinct cell populations of the hematopoietic system during development and in adult life. The W locus effects hematopoiesis through the erythroid lineages, mast cell lineages and stem cells, resulting in a macrocytic anemia which is lethal for homozygotes of the most severe W alleles, and a complete absence of connective tissue and mucosal mast cells.

Compositions

The protease activators and genetically manipulated cells described herein can be administered to an animal (e.g. a human) by any procedure available to one of skill in the art. Such activators and cells may be administered by intravascular, intravenous, intra-arterial, intraperitoneal, intraventricular infusion, infusion catheter, balloon catheter, bolus injection, direct application to tissue surfaces during surgery, or other convenient routes. The cells can be washed after collection, cultured in an appropriate medium to insure their viability and to enhance their numbers. The cells can also be cultured in the presence of growth factors such as G-CSF, GM-CSF, VEGF, SCF (c-kit ligand), buff, chemokines such as SDF-1, or interleukins such as interleukins 1 and 8. Prior to administration, the cells can be washed again, for example, in buffered physiological saline.

The volume of cells that is injected and the concentration of cells in the transplanted solution depend on the site of administration, the injury or disease to be treated, and the species of the host. Preferably about one-half to about five micro liters is injected at a time. The number of cells injected can vary, for example, about 10² to about 10¹⁰ or about 10⁴ to about 10⁹ cells can be injected at one time. While a single injection may be sufficient, multiple injections may also be used.

Protease activators can be administered with or without the cells of the invention. These protease activators can therefore be incorporated into pharmaceutical compositions that also contain endothelial precursor cells or bone marrow cells and that are suitable for administration to a mammal. Such compositions may also contain a pharmaceutically acceptable carrier.

As used herein, “pharmaceutically acceptable carrier” is intended to include any and all solvents, stabilizing agents, dispersion media, coatings, antibacterial and antifungal agents, isotonic agents, absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the cells or polypeptides provided herein, use thereof in the compositions is contemplated. Supplementary active compounds can also be incorporated into the compositions.

A pharmaceutical composition of the invention is formulated to be compatible with its intended route of administration. Examples of routes of administration include parenteral, intravenous, intradermal, subcutaneous, oral, inhalation, transdermal (topical), transmucosal, subdermal, subcutaneous, transdermal, or rectal. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a protease activator) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. While many applications may require non-oral administration of the compositions, oral administration is contemplated for compounds and peptides that are formulated in a manner that facilitates their retention of activity and their absorption through the gastrointestinal tract.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The protease activators and other compounds can also be prepared in the form of suppositories (e.g., with conventional suppository bases such as cocoa butter and other glycerides) or retention enemas for rectal delivery.

In one embodiment, the active compounds are prepared with carriers that will protect the protease activators and other compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Sustained-release preparations can be prepared. Suitable examples of sustained-release preparations include semipermeable matrices of solid hydrophobic polymers containing the polypeptide(s), which matrices are in the form of shaped articles, e.g., films, or microcapsules. Examples of sustained-release matrices include polyesters, hydrogels (for example, poly(2-hydroxyethyl-methacrylate), poly(vinylalcohol)), polylactide (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release pharmaceutical active agents over shorter time periods.

It is especially advantageous to formulate compositions in dosage unit form for ease of administration and uniformity of dosage. Dosage unit form as used herein refers to physically discrete units suited as unitary dosages for the subject to be treated; each unit containing a predetermined quantity of activator, cells and compounds calculated to produce the desired therapeutic effect in association with the required pharmaceutical carrier.

The pharmaceutical compositions can be included in a kit, e.g., in a container, pack, or dispenser together with instructions for administration.

The following examples are intended to illustrate the invention and should not be interpreted to limit it in any manner.

### EXAMPLE 1

### Material and Methods

This Example provides some of the materials and methods used for generating experimental results.

Animal Studies

SCID mice, age and sex-matched (7-8 weeks), weight (>20 g) were purchased from the Jackson Laboratory (Bar Harbor, Me.). A mouse line with a null mutation in the MMP-9 gene (MMP-9^{-/-}) was generated using the method of Vu et al. (1998) Cell 93, 411-22. In particular, MMP-9^{-/-} 129Sv mice were generated and used after at least eight back crosses to CD 1 mice. MMP-9^{+/+} 129Sv mice were obtained by back crossing MMP-9+1-129Sv mice with CDI mice. Mice were maintained in filtered germ-free air Thorensten units.

Administration of 5-FU. MMP-9^{-/-} and MMP-9^{+/+} mice were injected with the cell cycle cytotoxic agent 5-fluorouracil (5-FU) to deplete cycling hematopoietic cells. In particular, 250 mg/kg body weight 5-FU (Pharmacia & Upjohn Company) was administered intravenously (i.v.). At specified time points, mice were sacrificed. Femurs were flushed and bone marrow cells were used for cell cycle analysis or subjected to magnetic bead isolation (MACS) (Miltenyi Biotec). Lineage-negative bone marrow cells were isolated by MACS after labeling the cells with a depletion cocktail of lineage-specific monoclonal antibodies (mAbs), including CD5, CD45R, CD11b, Gr-1, TER119, 7/4 (Stem Cell Technology, Canada). Lin⁻ cells were MACS isolated with mAb against Sca-1. The separated Lin⁻Sca-1⁺bone marrow cells were >96% positive for Sca-1 (clone E13-161.7; PharMingen, San Diego) and >99% positive for c-Kit (clone 2B8, Bioscience). Less than 4% of the Lin⁻Sca-1⁺cells had staining for CD11b, CD3, B220 or Gr-1. For cell cycle analysis whole bone marrow cells of MMP-9^{-/-} and MMP-9^{+/+} mice were labeled with Sca-1-FITC. Lin⁻Sca-1⁺bone marrow cells were labeled with c-Kit-FITC. Labeled bone marrow cells and Lin⁻Sca-1⁺cells were fixed in ice-cold ethanol for 30 min. After RNAase treatment (Sigma, MO), cells were stained with propidium iodide (Molecular-Probes, Oregon). The DNA content of Sca-1 and Lin⁻Sca-1⁺c-Kit⁺ cells was determined by FACS (Coulter flow cytometer).

Mobilization of bone marrow-derived cells by adenovirus delivered factors. Plasma levels of VEGF, SDF-1 and KitL were elevated by using an adenovirus 5 (Ad)-derived E1a-, E3-deficient (E1ₐ⁻E3⁻E4⁺) vector with an expression cassette in the E1a-region containing the transgene cDNA and driven by the cytomegalovirus promoter/enhancer (Hattori et al., 2001b). Mice received the Ad-vector expressing SDF-1 (AdSDF-1) or no transgene (AdNull) at a concentration of 1×10⁹ plaque forming units (PFU) and Advector expressing sKitL (AdsKitL) or VEGF₁₆₅ (AdVEGF) at a concentration of 1.5×10⁸ PFU. The vector was injected i.v. as a single dose. Recombinant G-CSF (R&D Systems, MN) was administered subcutaneously daily from day 0-5 at 50 μg/kg body weight. Recombinant murine KitL (peproTech, NJ) was injected s.c. twice a day at 100 or 200 μg/kg body weight.

CGS 27023A, a potent inhibitor of MMP-9 (IC₅₀=8 nM), MMP-2 (IC₅₀=11 nM) and MMP-3 (IC₅₀=13 nM; Novartis, Basel, Switzerland), was injected at 60 mg/kg body weight s.c. every 3 days starting from day 0.

Bone Marrow Transplantation.

Bone marrow cells (3×10⁷/mouse) from 8-week-old MMP-9^{-/-} and MMP-9^{+/+} mice were injected i.v. into lethally irradiated (9.5 Gy from a 137CS γ-ray source) MMP-9^{-/-} and MMP-9^{+/+} mice. Sixteen days later, mice were treated with 5-FU as described above. Survival was monitored.

Hematopoietic/Endothelial Progenitor and Stem Cell Assays

Peripheral blood analysis. Blood was collected with capillary pipettes by retro-orbital bleeding and WBC counts were determined by using a hemocytometer. Peripheral blood mononuclear cells (PBMCs) were isolated from heparinized blood after centrifugation over a discontinuous gradient using Lympholyte-M (Cedarlane, Ontario, Canada).

Progenitor assay. Peripheral blood mononuclear cells (10⁵) were plated in Iscove's modified Dulbecco's medium (IMDM) containing 1.2% methylcellulose (Fisher Scientific, NJ), 30% FBS, 5×10⁻³M 2-mercaptoethanol 2 mM L-glutamine, 0.5 mM hemin (Sigma), murine KitL (20 ng/ml), human erythropoietin (6U/ml) and murine IL-3 (50 ng/ml). Colonies (>50 cells) were scored after 7 days (37° C., 5% CO₂).

CFU-S assay. Mobilized peripheral blood mononuclear cells (10⁵/mouse) of chemokine-treated SCID mice were injected i.v. into lethally irradiated syngeneic recipients (9 Gy). Spleens were removed 12 days later and fixed in Bouin's solution. The number of macroscopic spleen colonies was scored.

Bone marrow repopulating assay. Chemo/cytokine-mobilized PBMCs from SCID mice treated with/without MPI were collected on day 7. PBMCs from G-CSF-treated MMP-9^{-/-} and MMP-9^{+/+} mice were collected on day 5. Mobilized PBMCs (10⁵) were injected into lethally irradiated syngeneic animals (9.5 Gy). Survival was monitored.

Endothelial progenitor assay. Circulating bone marrow-derived CEPs were quantified by colony forming unit-endothelial cell (CFU-EC) as previously described (Hattori et al. 2001a). Mobilized PBMCs were cultured in M199 media (GIBCO-BRL, Gaithersburg, Md.) supplemented with 20% FBS (HyClone, UT), ECGF (30 μg/ml), human FGF-basic (Sigma) and heparin. After 5 weeks of culture, adherent cells were scored for colonies (>10 cells). Endothelial colonies were identified by metabolic uptake of DiI-Acetylated-LDL (1 μg/ml; PerImmune Inc, Rockville, Md.) at 37° C. for 5 hours, and visualized fluorescence microscopy. The number of VEGFR2⁺ cells was quantified by FACS using a Cy2-labeled mAb to VEGFR2 (clone DC101, ImClone, NY), (Hattori et al. 2001a).

In Vitro Assays

Murine bone marrow cultures. Murine bone marrow cells (1×10⁶) from MMP-9^{-/-} and MMP-9^{+/+} bone marrow cells isolated after treatment with/without 5-FU were placed in serum-free medium (X-VIVO-20) overnight. Culture supernatants were analyzed by Western blotting using murine mAb (1 μg/ml) to MMP-9 (clone 7-11C Oncogene Research-Products), anti-mouse IgG-conjugated to horseradish peroxidase (1:6000 dilution, Santa Cruz Biotechnology, CA) and visualized using ECL chemiluminescence (Amersham Pharmacia-Biotech).

Stromal cell line cultures. Stromal cells (MS-5) were cultured in serum-free medium, and treated with recombinant active MMP-9 (Oncogene, Boston, Mass.) or an MPI CGS 27023A for 24 hours.

Substrate zymography. Supernatants from human MACS-isolated CD34⁺ (5×10³) cells were collected after overnight incubation in serum-free medium with/without SDF-1 (150 ng/ml R&D-Systems) or VEGF (50 ng/ml, PeproTech). MMP activity was determined by gelatin zymography (Lane et al., 2000).

Immunoassay for chemokines and cytokines. Plasma levels of SDF-1, VEGF, and sKitL were measured using commercially available ELISA (R&D Systems).

Immunohistochemistry. Bone marrow sections were deparaffinized, rehydrated through an alcohol series immersed in 0.1% H₂O₂ and blocked using an Avidin and Biotin blocking kit (Vector, Burlingame, Calif.). For MMP-9 staining, the M.O.M. immunodetection kit (Vector) was used as recommended. Sections were incubated with MMP-9 mAb (clone 7-11C, Oncogene, MA) for 30 min, washed in PBS, incubated with a biotinylated horse anti-mouse IgG and incubated for 5 min with A&B reagents (Vector). Von Willebrand Factor (vWF) immunohistochemistry was performed on deparaffinized sections, treated with 3% H₂O₂, incubated with 0.05% Pronase E (Sigma). Sections were stained with anti-human vWF/HRP (Dako). Sections were developed with 3,3′-diaminobenzidine substrate and counterstained with eosin and hematoxylin.

In vitro transmigration through reconstituted basement membranes. CD34⁺ cells from PBMC of normal donors were isolated by MACS (Miltenyi Biotec) and showed a purity of >95% by FACS. CD34⁺ cells were added to 8 μm pore Matrigel-coated (BD) transwell inserts (Costar, Cambridge Mass.). Recombinant SDF-1 (100 ng/ml) was added to the lower chamber with/without metalloproteinase inhibitors (MPIs) 5-phenyl-1,10-phenanthroline (1 mM, Sigma), or CGS 27023A (1 nM, Novartis, Switzerland) which were added to both chambers. Transmigrated cells were quantified, placed in a clonogenic assay and in long-term cultures to measure the number of CAFC and LTC-IC (Jo et al., 2000).

Statistical analysis Results are expressed as mean±standard deviation. Data were analyzed using the unpaired two-tailed Student's t-test and the log rank test. P values of <0.05 were considered significant.

### EXAMPLE 2

This Example illustrates that bone marrow suppression with 5-FU results in apoptosis of actively cycling hematopoietic stem cells and progenitor cells, while sparing hematopoietic stem cells in the G₀ phase of the cell cycle. Moreover, as described herein MMP-9 expression is increased after bone marrow suppression.

Bone Marrow Suppression Induces MMP-9 Expression in Bone Marrow Cells

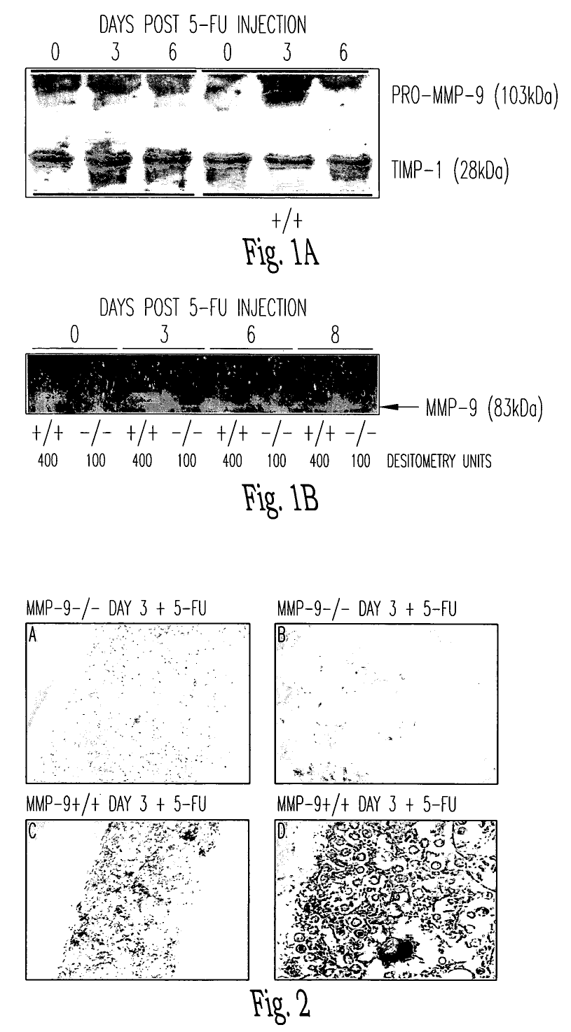

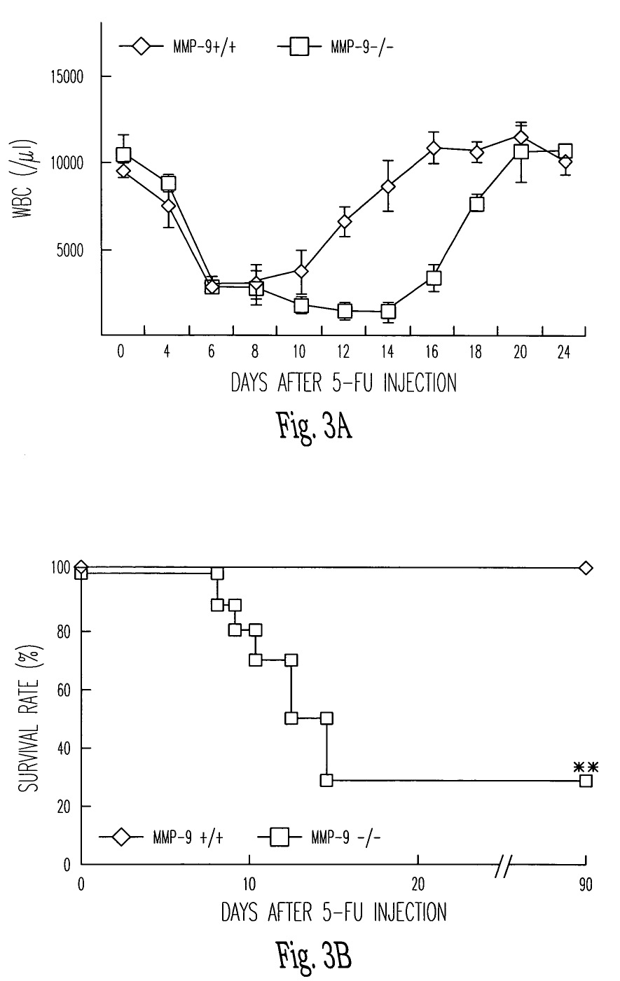

As shown in FIGS. 1A and 1B, expression of pro-MMP-9 and active MMP-9 increased in supernatants three days after 5-FU treatment of bone marrow cells from MMP-9 wild type animals. No significant change in tissue inhibitor of metalloproteinase-1 (TIMP-1) expression was observed. In bone marrow cells of untreated animals there was a small amount of MMP-9 (FIG. 1B), possibly derived from resident neutrophils (Murphy et al., 1982). Moreover, bone marrow hematopoietic and stromal cells expressed MMP-9 in vivo three days after treatment of wild type mice with 5-FU (FIGS. 2C-D). These data suggest that both pro-MMP-9 and MMP-9 are up-regulated in bone marrow cells after myelosuppression

MMP-9^{-/-} Mice Show Delayed Hematopoietic Recovery Following 5-FU Treatment.

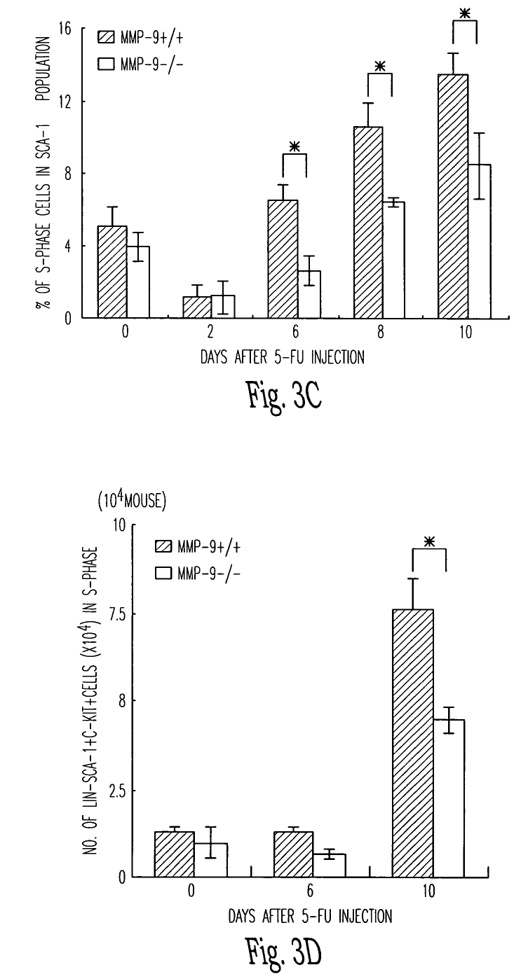

MMP-9 deficient mice were used to probe the role of MMP-9 in promoting hematopoietic stem cell recruitment and hematopoietic recovery after bone marrow suppression. Under steady state conditions, adult MMP-9^{-/-} and MMP-9^{+/+} mice have similar peripheral white blood cell (WBC) counts. Both MMP-9^{-/-} and MMP-9^{+/+} mice became leukopenic within six days after 5-FU treatment (FIG. 3A). However, in 5-FU-treated MMP-9^{-/-} mice, the recovery of white blood cell counts (FIG. 3A) and platelets (data not shown) was delayed by 8 days, compared to MMP-9^{+/+} mice. This prolonged delay in hematopoietic recovery resulted in the death of 72% of 5-FU-treated MMP-9^{-/-} mice (n=16, p<0.001) (FIG. 3B). In contrast, all of the MMP-9^{+/+} mice survived (FIG. 3B). These data indicate that MMP-9 plays a critical role in accelerating hematopoietic reconstitution.

Tests were then performed to determine whether the impaired hematopoietic recovery of MMP-9^{-/-} mice was due to an alteration in the stem/progenitor cell cycling status. Hematopoietic cells phenotypically marked as Lin⁻Sca-1⁺c-Kit⁺ comprise a large percentage of long-term repopulating hematopoietic stem cells (Cheshier et al., 1999). Under steady state conditions, the percentage and the total number of Sca-1⁺(FIG. 3C) and Lin⁻Sca-1⁺c-Kit⁺ cells (FIG. 3D) in the S-phase of the cell cycle was not significantly different in MMP-9^{+/+} and MMP-9^{-/-} mice. However, following 5-FU myeloablation the number of these cells entering S-phase was impaired in MMP-9^{-/-} mice. These data indicate that although during steady state hematopoiesis there is no substantial difference in the number of cycling repopulating cells, in the recovery phase after myelosuppression, lack of MMP-9 results in a diminished recruitment of cycling cells leading to profound bone marrow hypocellularity.

Restoration of Myeloid and Megakaryocytic Lineages is Impaired in MMP-9^{-/-} Mice

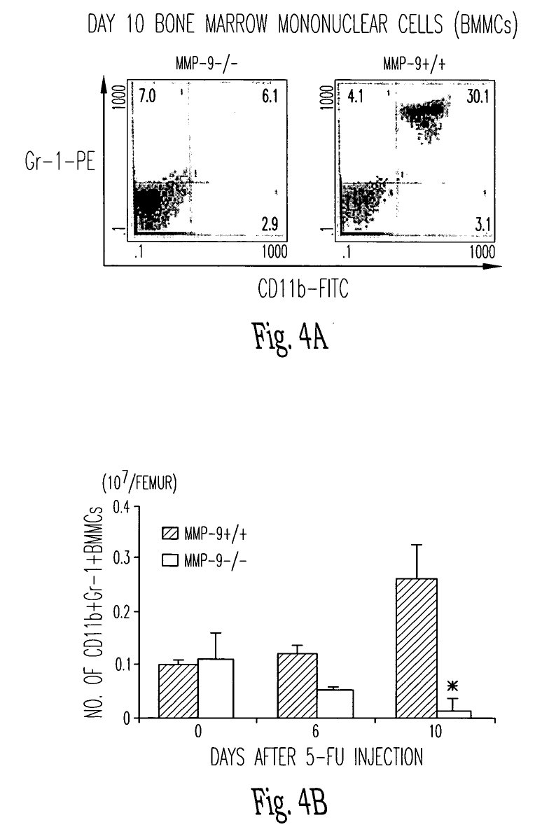

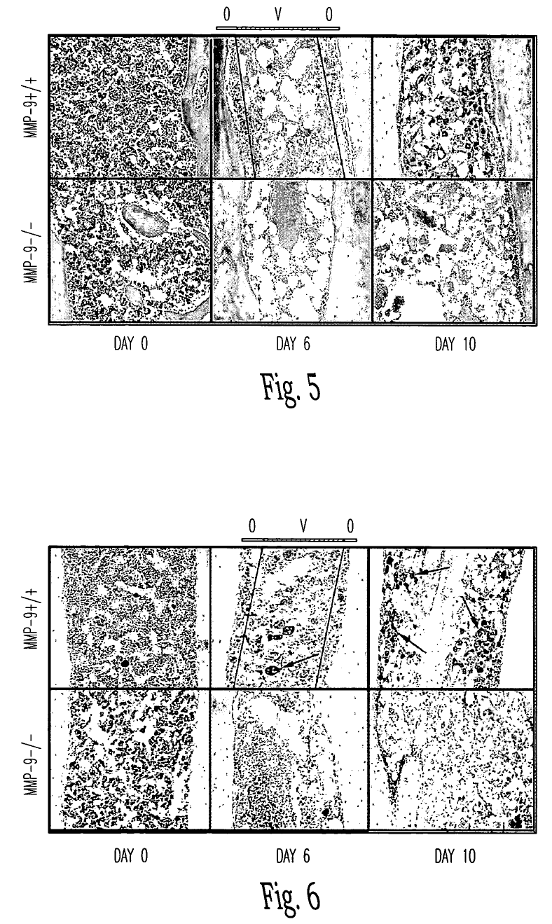

On day 6 after 5-FU treatment, bone marrow cell numbers increased in MMP-9^{+/+} mice (FIG. 3C). Moreover, hematopoietic cell clusters were seen in close contact to the bone “osteoblastic zone (O)”, followed by a shift of cells moving towards the blood vessel-enriched “vascular zone (V)” on day 10 (FIG. 5). However, in the bone marrow of MMP-9^{-/-} animals, there was a paucity of hematopoietic cell clusters in both the osteoblastic and vascular zones, even on day 10 after 5-FU treatment. By day 10, the proportion of differentiated CD11b⁺Gr-1⁺ myeloid (FIGS. 4A-B) and vWF⁺amegakaryocytic precursor cells (FIG. 6) in the bone marrow of 5-FU-treated MMP-9^{-/-} mice was dramatically reduced. The impaired recruitment of repopulating stem and progenitor cells into the vascular zone and the lack of differentiation into myeloid and megakaryocytic lineages in MMP-9^{-/-} mice led to the hypothesis that MMP-9 may exert its effects through the release of a stem cell-active cytokine.

Release of Soluble KitL (sKitL) is Impaired in MMP-9^{-/-} Mice After Bone Marrow Suppression

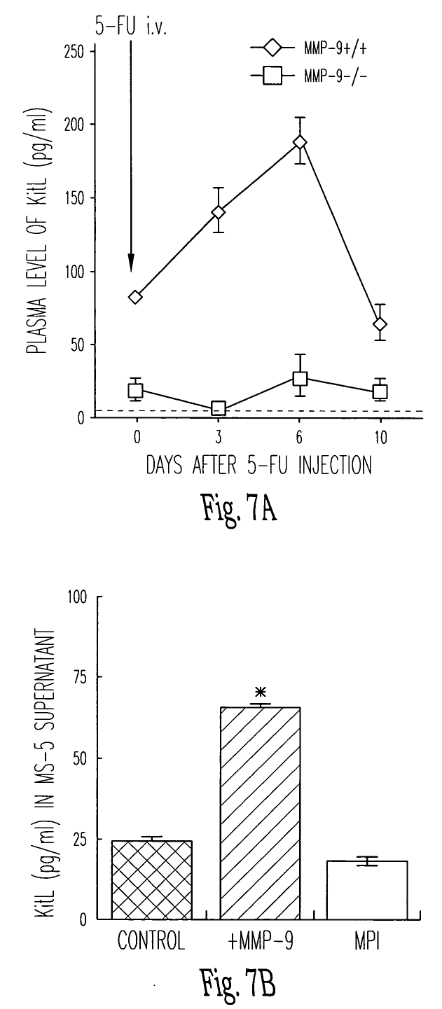

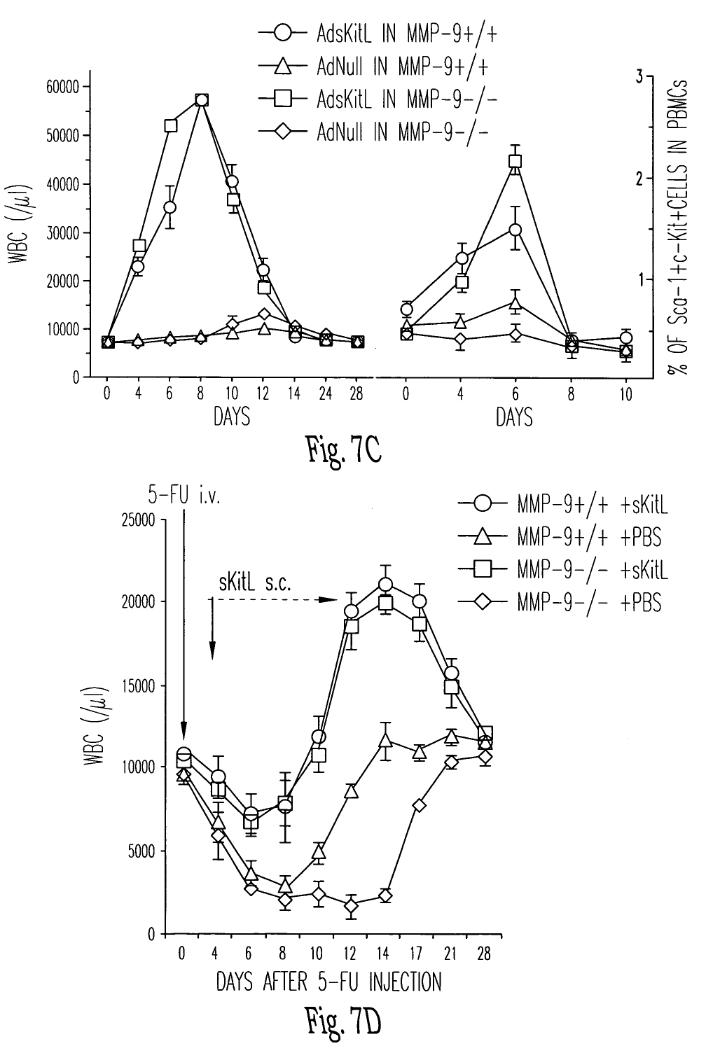

Among the known stem cell-active chemokines and cytokines, KitL conveys signals that modulate survival, adhesion and motility of c-Kit⁺ hematopoietic stem cells and endothelial cells. sKitL is expressed as a membrane (mKitL) form, and can be cleaved to a soluble form (sKitL) (Huang et al., 1992). Indeed, plasma levels of sKitL increased 3 fold during the hematopoietic regeneration phase in MMP-9^{+/+} mice, peaking at 6 days when there was active proliferation in the bone marrow. In contrast, baseline sKitL plasma levels were very low in MMP-9^{-/-} mice and did not increase following bone marrow ablation (FIG. 7A). These data suggest that MMP-9 promotes rapid release of sKitL, facilitating bone marrow recovery.

Unstimulated bone marrow stromal cells express both mKitL and MMP-9 (Heinrich et al., 1993; Marquez-Curtis et al., 2001). Although MMP-9 was present at low levels in the supernatants of the unstimulated murine bone marrow stromal cell line MS-5, which expresses mKitL, addition of recombinant MMP-9 rapidly promoted the release of sKitL. This shedding was blocked by a synthetic metalloproteinase inhibitor (MPI) (FIG. 7B). These data suggest that MMP-9 can effectively promote the release of sKitL from stromal cells.

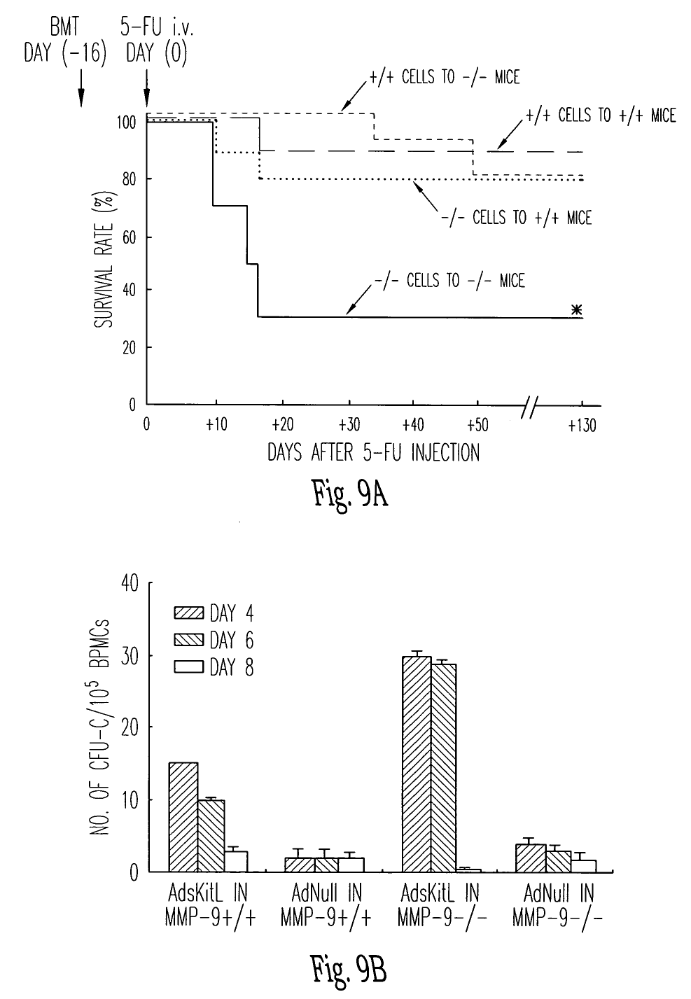

However, MMP-9 expression is up-regulated in both hematopoietic and stromal compartments of the bone marrow post-myeloablation (FIG. 2). To identify which cellular compartment is more critical in providing bioactive MMP-9 following myelosuppression, chimeric mice were developed by transplanting bone marrow cells from MMP-9^{-/-} mice into lethally irradiated MMP-9^{+/+} recipients resulting in chimeric mice in which hematopoietic cells were MMP-9 deficient. Transplantation and engraftment of bone marrow from MMP-9^{+/+} into MMP-9^{-/-} mice resulted in mice with MMP-9 deficient stroma (FIG. 9A). As controls, MMP-9^{+/+} bone marrow cells were transplanted into lethally irradiated MMP-9^{+/+} mice and MMP-9^{-/-} bone marrow cells into lethally irradiated MMP-9^{-/-} mice. All mice showed engraftment after 16 days as determined by WBC counts. The majority of MMP-9^{+/+} recipients transplanted with either MMP-9^{-/-} or MMP-9^{+/+} bone marrow or MMP-9^{-/-} recipients transplanted with MMP-9^{+/+} bone marrow survived 5-FU myelosuppression. As expected, high mortality was observed in the MMP-9^{-/-} group transplanted with MMP-9^{-/-} bone marrow cells (FIG. 9a). These data indicate that MMP-9 expressed either by stromal or hematopoietic cells is sufficient to support hematopoiesis after myelosuppression.

Exogenous sKitL Restores Hematopoietic Recovery and Mobilization in MMP-9^{-/-} Mice

Under steady state conditions, elevated sKitL levels delivered by adenoviral vector expressing sKitL (AdsKitL) increased WBC in both MMP-9^{-/-} and MMP-9^{+/+} mice (FIG. 7C). Progenitor mobilization determined by either the frequency of mobilized Sca-1⁺c-Kit⁺ cells (FIG. 7C) or colony-forming unit cells (CFU-Cs) in the peripheral blood (Supplement, FIG. 9B) was enhanced in the MMP-9^{-/-} mice after AdsKitL introduction.

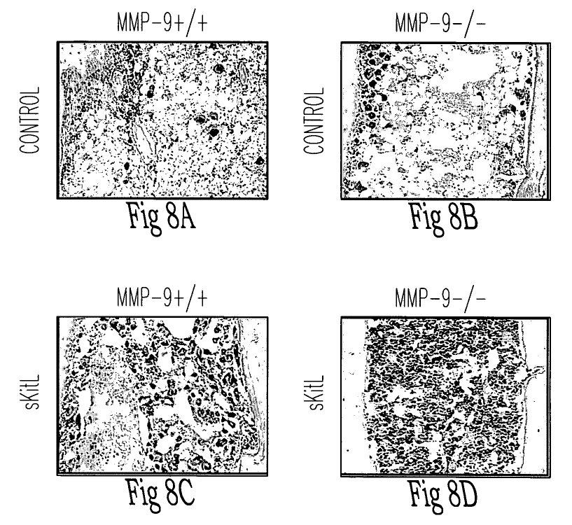

MMP-9 augments the release of sKitL. Tests were then performed to ascertain whether the delayed hematopoietic recovery seen in MMP-9^{-/-} mice could be restored directly by exogenous recombinant sKitL. Treatment of MMP-9^{-/-} mice with recombinant sKitL following 5-FU treatment resulted in rapid recovery of WBC (FIG. 7D). In addition, sKitL treatment following 5-FU bone marrow suppression resulted in a rapid increase in bone marrow cellularity and hematopoietic reconstitution in MMP-9^{-/-} mice (FIG. 8) and restored survival of MMP-9^{-/-} mice (increasing survival 83% compared to 28% observed in the PBS group, n=10 in each group). These data suggest that MMP-9-mediated release of sKitL is essential for promoting stem cell differentiation, accelerating hematopoietic reconstitution following bone marrow ablation.

Chemokines Released upon Bone Marrow Suppression Promote MMP-9 Expression

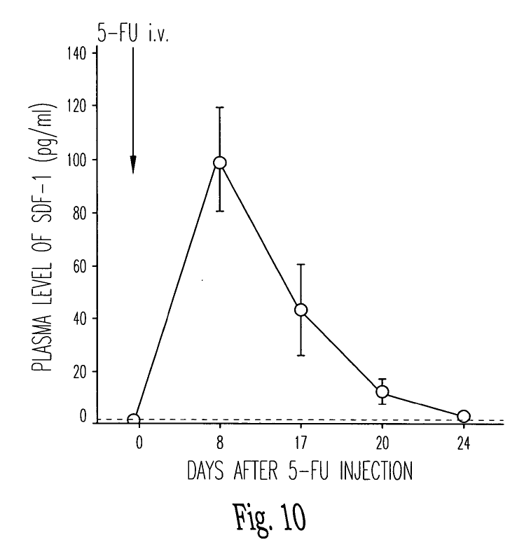

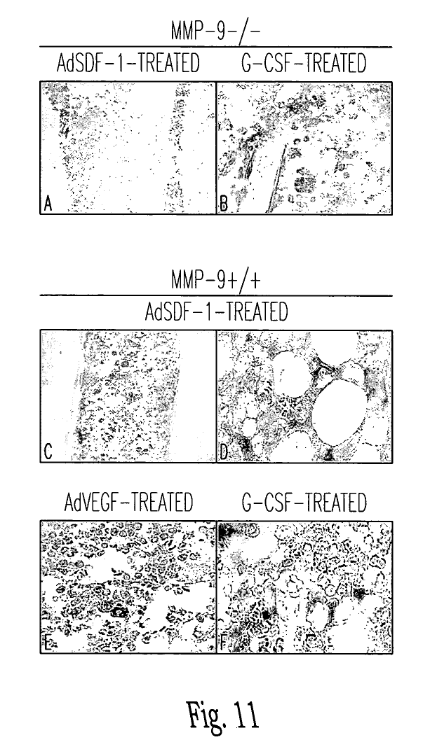

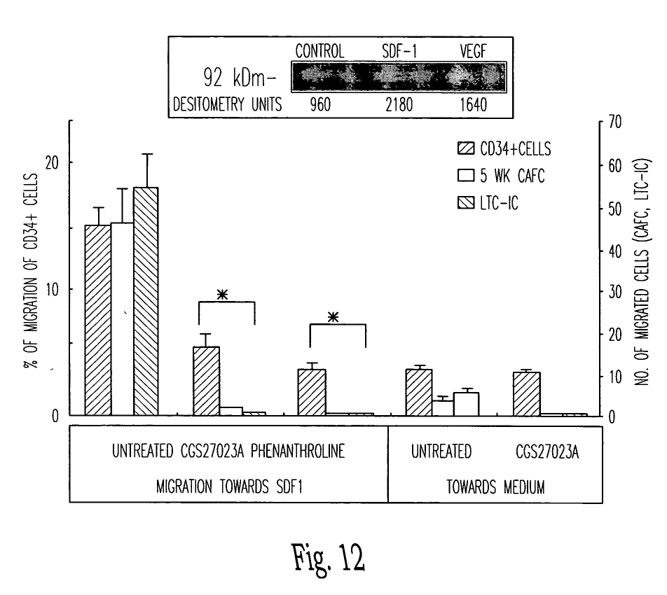

One clue as to what regulates MMP-9 came from the observation that the chemokine stromal cell-derived factor-1 (SDF-1) increased after myelosuppression (Ponomaryov et al., 2000). Plasma levels of SDF-1 increased following 5-FU treatment, peaking on day 8 (FIG. 10). These data suggest that rapid elevation of chemo/cytokine levels after bone marrow ablation contribute to MMP-9 up-regulation, setting the stage for hematopoietic stem cell recruitment. To determine whether chemokines or cytokines induce MMP-9 expression in vivo, SDF-1, VEGF and G-CSF were separately introduced into wild type mice. The bone marrow of mice treated with SDF-1, VEGF or G-CSF showed increased immunoreactive MMP-9 in stromal and hematopoietic cells (FIGS. 11b-f). These studies raised the possibility that chemokine- or cytokine-induced MMP-9 activation mediates mobilization of hematopoietic cells. In a transwell migration assay, SDF-1 and VEGF did stimulate the release of pro-MMP-9 and induce migration of human CD34⁺ progenitor and stem cells (5 week cobblestone forming cells (CAFC) and long-term culture initiating cells (LTC-IC)) (FIG. 12). The migration of CD34⁺ cells was completely blocked by addition of metalloproteinase inhibitors CGS 27023A and 5-phenyl-1,10-phenanthroline. Direct incubation of CD34⁺ cells with metalloproteinase inhibitors in suspension culture did not alter their proliferation in CAFC or LTC-IC assays (data not shown). These data suggest that chemokines induce the functional expression of MMP-9 on CD34⁺ cells and their migration.

Chemokine-Induced Mobilization of Bone Marrow Repopulating Cells is Impaired in MMP-9 Deficient Mice

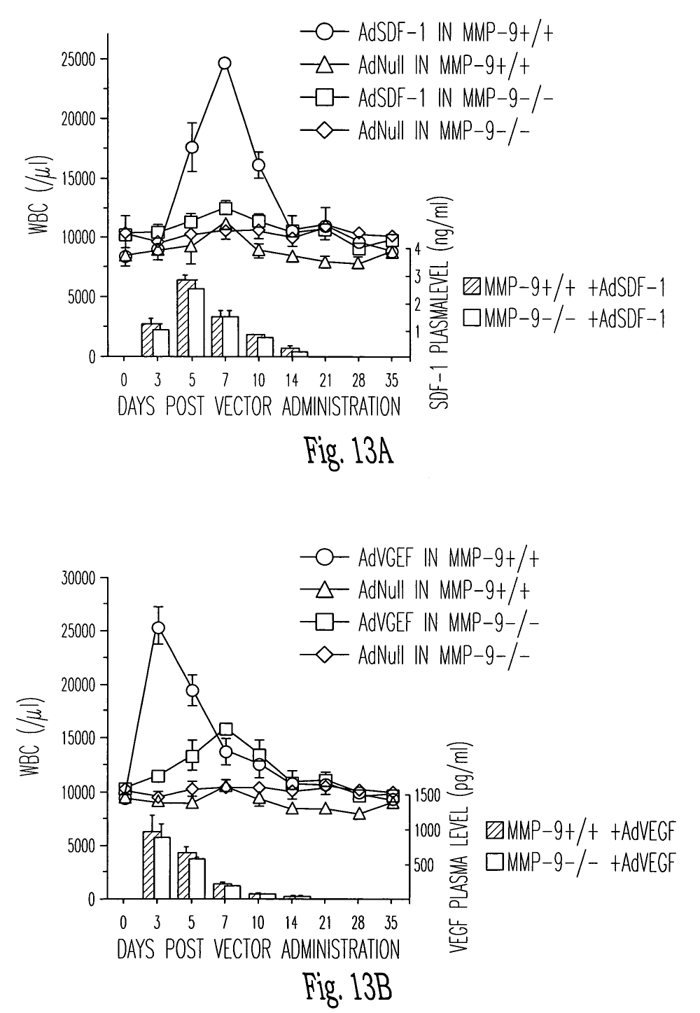

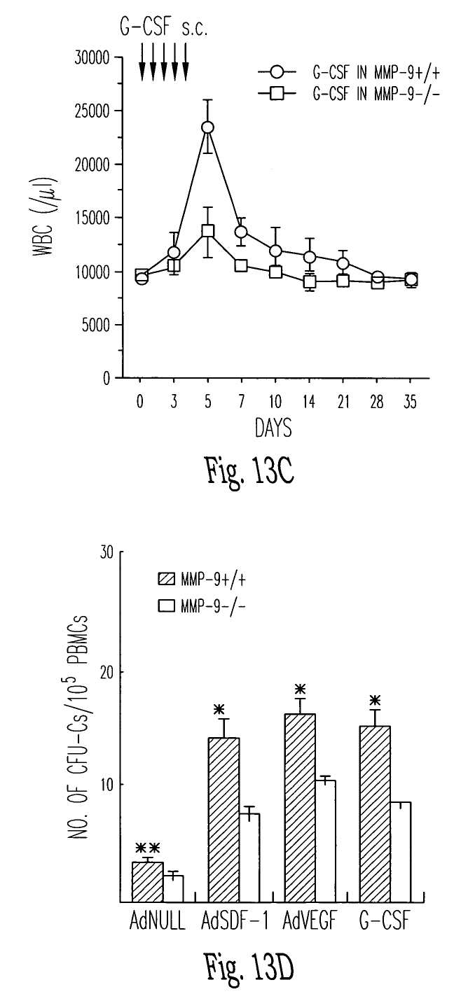

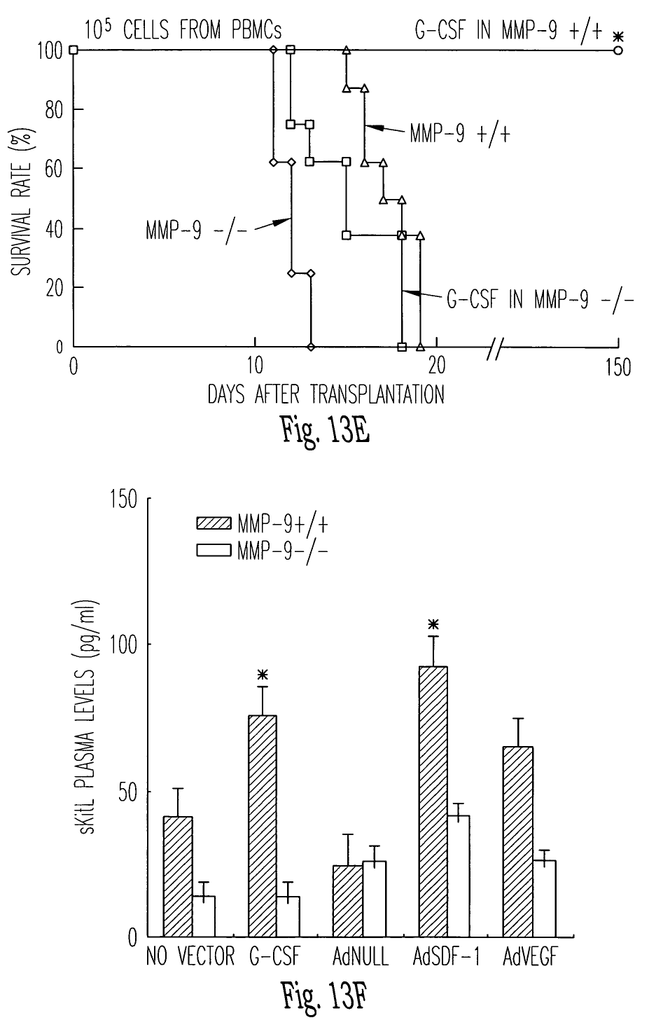

Plasma elevation of SDF-1, VEGF and G-CSF in MMP-9^{+/+} mice, but not in MMP-9^{-/-} mice, mobilized mature white blood cells (FIGS. 13A-C) and hematopoietic progenitors (CFU-Cs) (FIG. 13D). The mobilization of hematopoietic cells into the circulation followed the kinetics of plasma elevation for these chemokines (FIG. 13A, B). Next, tests were performed to determine if MMP-9 plays a role in mobilization of cells with repopulating capacity by transplanting peripheral blood mononuclear cells (PBMCs) mobilized by G-CSF as the source of bone marrow repopulating cells. Lethally irradiated syngeneic mice that received transplanted G-CSF-mobilized PBMCs harvested from MMP-9^{+/+} mice 5 days after chemokine treatment showed long-term donor cell engraftment and survival, whereas those transplanted with G-CSF-mobilized PBMCs from MMP-9^{-/-} mice died during the 20 days allowed for engraftment (FIG. 13E). These data show that MMP-9 plays a role in mobilization of bone marrow repopulating cells.

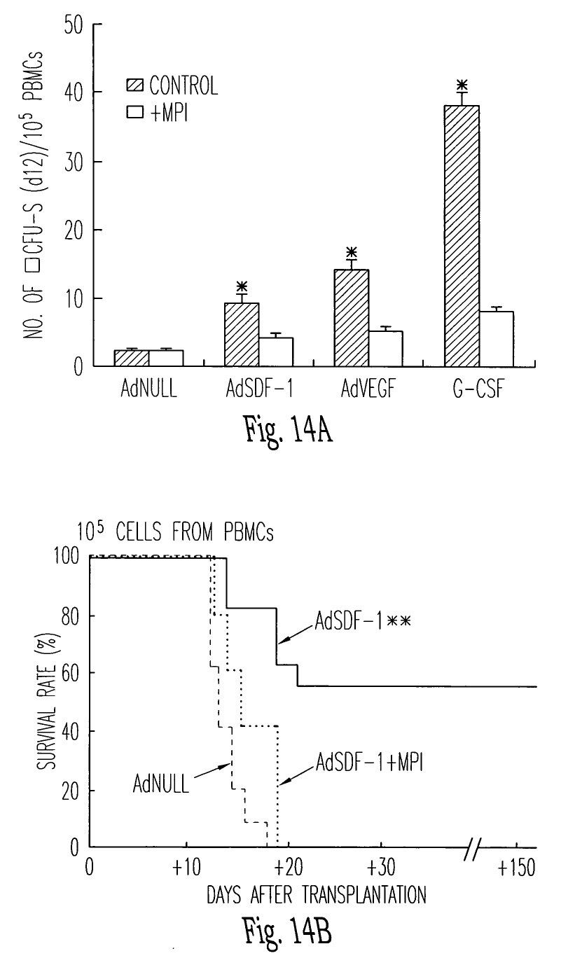

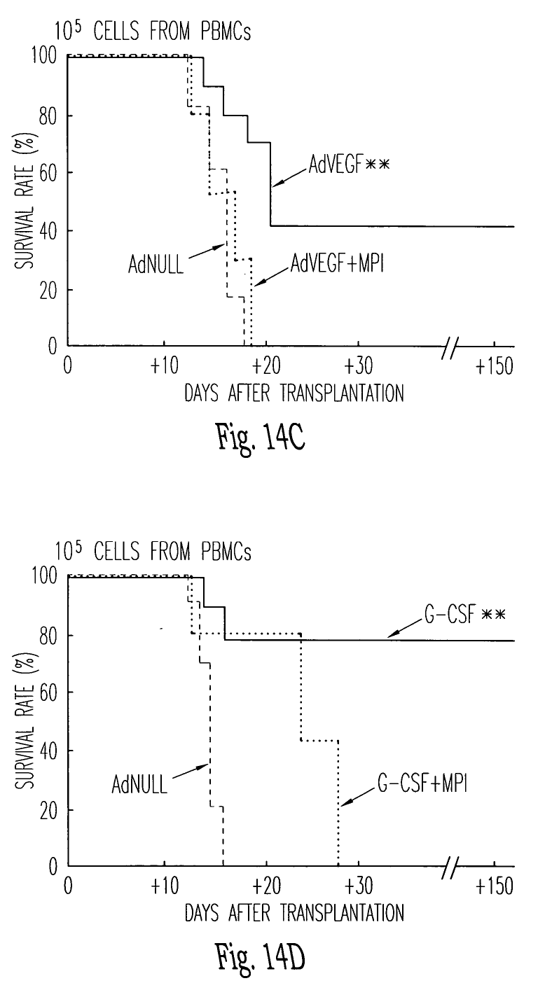

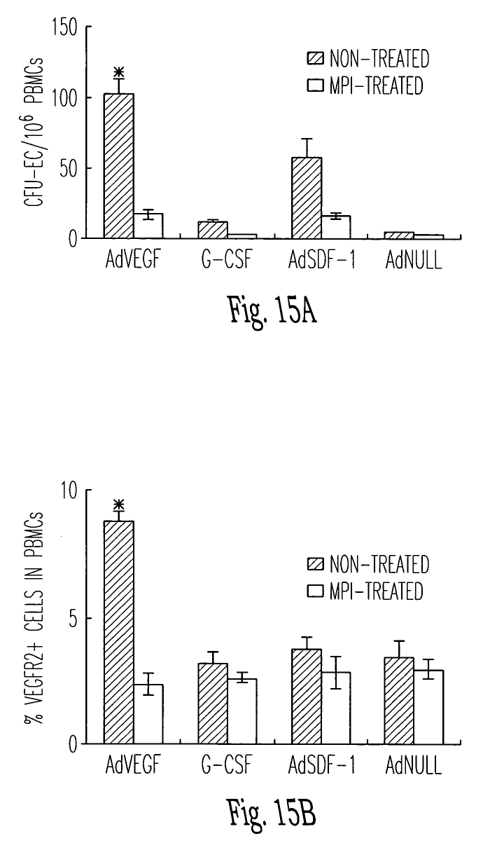

The need for chemokine-induced mobilization on MMP activity was confirmed by administrating a synthetic metalloproteinase inhibitor in vivo which also decreased white blood cell counts and blocked chemokine-induced mobilization in MMP-9 competent mice (data not shown). Importantly, mobilization of hematopoietic cells with short-term repopulating potential (CFU-S) was completely blocked in metalloproteinase inhibitor-treated mice (FIG. 14a). Lethally irradiated syngeneic mice transplanted with SDF-1-, VEGF-, and G-CSF-mobilized PBMCs showed long-term engraftment in 55%, 40% and 80% of these mice, respectively (FIG. 14b,c,d). Similar to MMP-9^{-/-} mice, transplantation of PBMCs from MPI-treated mice failed to reconstitute hematopoiesis in lethally irradiated recipients and all recipient mice died within 19 days.

SDF-1 and VEGF Increase sKitL Plasma Levels

If one role for MMP-9 in the post-myelosuppression process is the release of sKitL, then impaired hematopoietic cell mobilization following SDF-1 and VEGF treatment of MMP-9^{-/-} mice could be due to their inability to produce sKitL. Indeed, increased plasma sKitL levels were observed in MMP-9^{+/+} mice after treatment with SDF-1, VEGF and G-CSF as compared to untreated animals (FIG. 13F). This chemokine- or cytokine-induced up-regulation of sKitL was impaired in MMP-9^{-/-} mice. These results highlight the importance of MMP-9 induced sKitL release for hematopoietic recovery.

Mobilization of Bone Marrow-Derived Endothelial Progenitors is MMP-9-Dependent

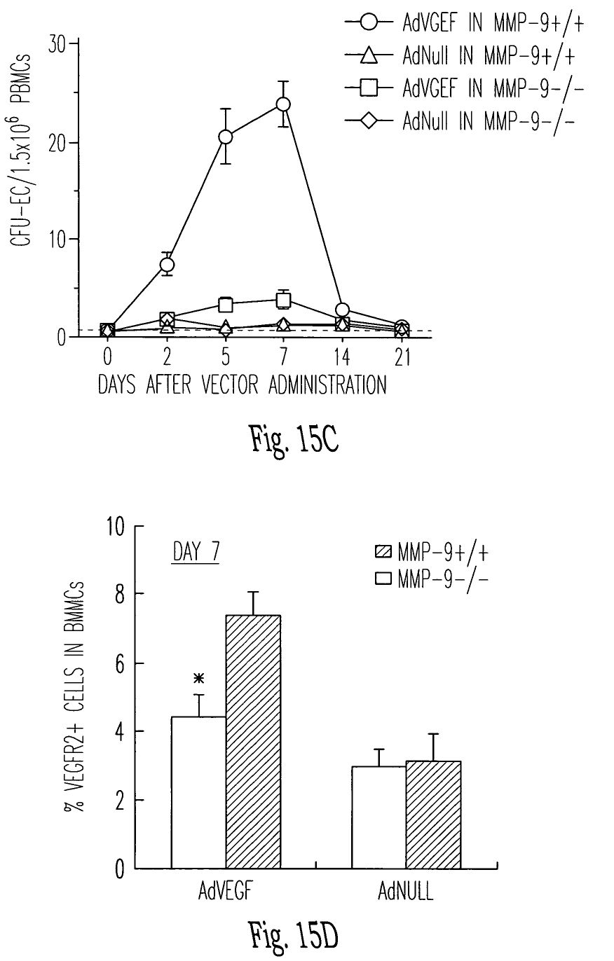

c-Kit is expressed on bone marrow-derived progenitors that can give rise to cardiac muscle, skeletal muscle and endothelial cells. Therefore, tests were performed to determine if MMP-9 plays a more general role in regulating other tissue-specific stem cells. Elevation of plasma levels of SDF-1 and VEGF in wild type mice increased the mobilization of circulating endothelial progenitors (CEPs) as assessed by the generation of late outgrowth endothelial cell colony forming units (CFU-EC) and expression of VEGF-receptor-2 (VEGFR2) (FIG. 15A, B). In contrast, VEGF did not mobilize CFU-EC or VEGFR2⁺ cells into the circulation of wild type mice treated with metalloproteinase inhibitors (FIG. 1SA, B). Similarly, VEGF increased the number of CFU-EC in the peripheral blood (FIG. 15C) and VEGFR2⁺ cells in the bone marrow of MMP-9^{+/+} but not in MMP-9^{-/-} mice (FIG. 15D). Collectively, these results suggest that MMP-9 activation is required for the recruitment and mobilization of bone marrow-derived CEPs.

### REFERENCES

Bergers, G., Brekken, R., McMahon, G., Vu, T. H., Itoh, T., Tamaki, K., Tanzawa, K., Thorpe, P., Itohara, Werb, Z., and Hanahan, D. (2000). Matrix metalloproteinase-9 triggers the angiogenic switch during carcinogenesis. Nat Cell Biol 2, 737-44.

Blau, H. M., Brazelton, T. R., and Weimann, J. M. (2001). The evolving concept of a stem cell: entity or function? Cell 105, 829-41.

Cheng, T., Rodrigues, N., Shen, H., Yang, Y., Dombkowski, D., Sykes, M., and Scadden, D. T. (2000). Hematopoietic stem cell quiescence maintained by p21cip1/waf1. Science 287, 1804-8.

Cheshier, S. H., Morrison, S. J., Liao, X., and Weissman, I. L. (1999). In vivo proliferation and cell cycle kinetics of long-term self-renewing hematopoietic stem cells. Proc Natl Acad Sci USA 96 3120-5.

Coussens, L. M., Tinkle, C. L., Hanahan, D., and Werb, Z. (2000). MMP-9 supplied by bone marrow-derived cells contributes to skin carcinogenesis. Cell 103, 481-90.

Engsig, M. T., Chen, Q. J., Vu, T. H., Pedersen, A. C., Therkidsen, B., Lund, L. R., Henriksen, K., Lenhard, T., Foged, N. T., Werb, Z., and Delaisse, J. M. (2000). Matrix metalloproteinase 9 and vascular endothelial growth factor are essential for osteoclast recruitment into developing long bones. J Cell Biol 151, 879-90.

Hattori, K., Dias, S., Heissig, B., Hackett, N. R., Lyden, D., Tateno, M., Hicklin, D. J., Zhu, Z., Witte, L. Crystal, R. G., Moore, M. A., and Rafii, S. (2001a). Vascular endothelial growth factor and angiopoietin stimulate postnatal hematopoiesis by recruitment of vasculogenic and hematopoietic stem cells. J Exp Med 193, 1005-14.

Hattori, K., Heissig, B., Tashiro, K., Honjo, T., Tateno, M., Shieh, J. H., Hackett, N. R., Quitoriano, M. S. Crystal, R. G., Rafii, S., and Moore, M. A. (2001b). Plasma elevation of stromal cell-derived factor-1 induces mobilization of mature and immature hematopoietic progenitor and stem cells. Blood 97, 3354-60.

Hattori, K., Hirano, T., Ushiyama, C., Miyajima, H., Yamakawa, N., Ebata, T., Wada, Y., Ikeda, S., Yoshino, K., Tateno, M., Oshimi, K., Kayagaki, N., Yagita, H., and Okumura, K. (1997). A metalloproteinase inhibitor prevents lethal acute graft-versus-host disease in mice. Blood 90, 542-8.

Heinrich, M. C., Dooley, D. C., Freed, A. C., Band, L., Hoatlin, M. E., Keeble, W. W., Peters, S. T., Silvey, K. V., Ey, F. S., Kabat, D., and et al. (1993). Constitutive expression of steel factor gene by human stromal cells. Blood 82, 771-83.

Huang, E. J., Nocka, K. H., Buck, J., and Besmer, P. (1992). Differential expression and processing of two cell associated forms of the kit-ligand: KL-1 and KL-2. Mol Biol Cell 3, 349-62.

Jo, D. Y., Rafii, S., Hamada, T., and Moore, M. A. (2000). Chemotaxis of primitive hematopoietic cells in response to stromal cell-derived factor-1. J Clin Invest 105 101-11.

Krause, D. S., Theise, N. D., Collector, M. I., Henegariu, O., Hwang, S., Gardner, R., Neutzel, S., and Sharkis, S. J. (2001). Multi-organ, multi-lineage engraftment by a single bone marrow-derived stem cell. Cell 105, 369-77.

Kridel, S. J., Chen, E., Kotra, L. P., Howard, E. W., Mobashery, S., and Smith, J. W. (2001). Substrate hydrolysis by matrix metalloproteinase-9. J Biol Chem 276, 20572-8.

Lane, W. J., Dias, S., Hattori, K., Heissig, B., Choy, M., Rabbany, S. Y., Wood, J., Moore, M. A., and Rafii S. (2000). Stromal-derived factor 1-induced megakaryocyte migration and platelet production is dependent on matrix metalloproteinases. Blood 96, 4152-9.

Levesque, J. P., Takamatsu, Y., Nilsson, S. K., Haylock, D. N., and Simmons, P. J. (2001). Vascular cell adhesion molecule-1 (CDI06) is cleaved by neutrophil proteases in the bone marrow following hematopoietic progenitor cell mobilization by granulocyte colony-stimulating factor. Blood 98, 1289-97.

Long, M. W., Briddell, R., Walter, A. W., Bruno, E., and Hoffman, R. (1992). Human hematopoietic stem cell adherence to cytokines and matrix molecules. J Clin Invest 90, 251-5.

Longley, B. J., Tyrrell, L., Ma, Y., Williams, D. A., Halaban, R., Langley, K., Lu, H. S., and Schechter, N. M. (1997). Chymase cleavage of stem cell factor yields a bioactive, soluble product. Proc Natl Acad Sci USA 94, 9017-21.

Lyden, D., Hattori, K., Dias, S., Costa, C., Blaikie, P., Butros, L., Chadburn, A., Keissig, B., Marks, W., Witte, L., Wu, Y., Hicklin, D., Zhu, Z., Hackett, N. R., Crystal, R. G., Moore, M. A., Hajjar, K. A., Manova, K., Benezra, R., and Rafii, S. (2001). Impaired recruitment of bone-marrow-derived endothelial and hematopoietic precursor cells blocks tumor angiogenesis and growth. Nat Med 7, 1194-1201.

Marquez-Curtis, L. A., Dobrowsky, A., Montano, J., Turner, A. R., Ratajczak, J., Ratajczak, M. Z., and Janowska-Wieczorek, A. (2001). Matrix metalloproteinase and tissue inhibitors of metalloproteinase secretion by hematopoietic and stromal precursors and their production in normal and leukaemic long-term marrow cultures. Br J Haematol 115, 595-604.

Masure, S., Paemen, L., Van Aelst, I, Fiten, P., Proost, P., Billiau, A., Van Damme, J., and Opdenakker, G. (1997). Production and characterization of recombinant active mouse gelatinase B from eukaryotic cells and in vivo effects after intravenous administration. Eur J Biochem 244, 21-30.

Morrison, S. J., Wandycz, A. M., Hemmati, H. D., Wright, D. E., and Weissman, I. L. (1997a). Identification of a lineage of multipotent hematopoietic progenitors. Development 124, 1929-39.

Morrison, S. J., Wright, D. E., and Weissman, I. L. (1997b). Cyclophosphamide/granulocyte colony-stimulating factor induces hematopoietic stem cells to proliferate prior to mobilization. Proc Natl Acad Sci USA 94, 1908-13.

Ohta, H., Yomogida, K., Dohmae, K., and Nishimune, Y. (2000). Regulation of proliferation and differentiation in spermatogonial stem cells: the role of c-kit and its ligand SCF. Development 127, 2125-31.

Orlic, D., Kajstura, J., Chimenti, S., Jakoniuk, I., Anderson, S. M., Li, B., Pickel, J., McKay, R., Nadal-Ginard, B., Bodine, D. M., Leri, A., and Anversa, P. (2001). Bone marrow cells regenerate infarcted myocardium. Nature 410, 701-5.

Palmer, T. D., Willhoite, A. R., and Gage, F. H. (2000). Vascular niche for adult hippocampal neurogenesis. J Comp Neurol 425, 479-94.

Papayannopoulou, T., Priestley, G. V., and Nakamoto, B. (1998). Anti-VLA4/VCAM-1-induced mobilization requires cooperative signaling through the kit/mkit ligand pathway. Blood 91, 2231-9.

Peichev, M., Naiyer, A. J., Pereira, D., Zhu, Z., Lane, W. J., Williams, M., Oz, M. C., Hicklin, D. J., Witte, L., Moore, M. A., and Rafii, S. (2000). Expression of VEGFR-2 and AC133 by circulating human CD34(+) cells identifies a population of functional endothelial precursors. Blood 95, 952-8.

Ponomaryov, T., Peled, A., Petit, I., Taichman, R. S., Habler, L., Sandbank, J., Arenzana-Seisdedos, F. Magerus, A., Caruz, A., Fujii, N., Nagler, A., Lahav, M., Szyper-Kravitz, M., Zipori, D., and Lapidot, T. (2000). Induction of the chemokine stromal-derived factor-1 following DNA damage improves human stem cell function. J Clin Invest 106, 1331-9.

Pruijt, J. F., Fibbe, W. E., Laterveer, L., Pieters, R. A., Lindley, 1. J., Paemen, L., Masure, S., Willemze, R. and Opdenakker, G. (1999). Prevention of interleukin-8-induced mobilization of hematopoietic progenitor cells in rhesus monkeys by inhibitory antibodies against the metalloproteinase gelatinase B (MMP-9). Proc Natl Acad Sci USA 96, 10863-8.

Reya, T., Morrison, S. J., Clarke, M. F., and Weissman, I. L. (2001). Stem cells, cancer, and cancer stem cells. Nature 414, 105-11.

Tajima, Y., Moore, M. A., Soares, V., Ono, M., Kissel, H., and Besmer, P. (1998). Consequences of exclusive expression in vivo of Kit-ligand lacking the major proteolytic cleavage site. Proc Natl Acad Sci U SA 95, 11903-8.

Vu, T. H., Shipley, J. M., Bergers, G., Berger, J. E., Helms, J. A., Hanahan, D., Shapiro, S. D., Senior, R. M. and Werb, Z. (1998). MMP-9/gelatinase B is a key regulator of growth plate angiogenesis and apoptosis of hypertrophic chondrocytes. Cell 93, 411-22.

Vu, T. H., and Werb, Z. (2000). Matrix metalloproteinases: effectors of development and normal physiology. Genes Dev 14, 2123-33.

Xie, T., and Spradling, A. C. (2000). A niche maintaining germ line stem cells in the Drosophila ovary. Science 290, 328-30.

All publications and patents are incorporated by reference herein, as though individually incorporated by reference.

The foregoing specification, including the specific embodiments and examples, is intended to be illustrative of the present invention and is not to be taken as limiting. Numerous variations and modifications can be effected without departing from the true spirit and scope of the present invention.

What is claimed:

1. A method for recruitment of adult stem cells in an animal comprising administering to the animal a protease or an activator of a protease, wherein the recruitment translocates an endogenous population of quiescent non-cycling stem cells to a permissive vascular zone in the animal so that the stem cells can proliferate, self-renew, differentiate or mobilize to a target site.

2. The method of claim 1, wherein the target site is an injured tissue, a diseased tissue, a regenerating organ, a developing organ or bone.

3. The method of claim 1, wherein the animal has been subjected to myelosuppressive stress.

4. The method of claim 1, wherein the target site is blood, bone marrow, cardiac tissue, hepatic tissue, lung tissue, kidney tissue, muscle tissue, neuronal tissue, vascular tissue or a combination thereof.

5. The method of claim 1, wherein the stem cells are hematopoietic stem cells, endothelial stem cells, hepatic stem cells, neuronal stem cells, muscle stem cells or a combination thereof.

6. The method of claim 1, wherein the protease is a matrix metalloproteinase, a collagenase, a gelatinase, a stromelysin, a matrilysin, a metalloelastase, or a membrane-type matrix metalloproteinase.

7. The method of claim 1, wherein the protease is matrix metalloproteinase-1, matrix metalloproteinase-2, matrix metalloproteinase-3, matrix metalloproteinase-4, matrix metalloproteinase-4, matrix metalloproteinase-5, matrix metalloproteinase-6, matrix metalloproteinase-7, matrix metalloproteinase-8, and matrix metalloproteinase-9, matrix metalloproteinase-10, matrix metalloproteinase-11, matrix metalloproteinase-12, matrix metalloproteinase-13, matrix metalloproteinase-14 or a combination thereof.

8. The method of claim 1, wherein the protease is matrix metalloproteinase-9.

9. The method of claim 1, wherein the activator is interleukin-1, thombopoietin, G-CSF, GMCSF, SDF-1, or fibroblast growth factor-4.

10. A method for increasing KitL expression in an animal comprising administering to the animal a protease or an activator of a protease.

11. The method of claim 10, wherein the protease is a matrix metalloproteinase, a collagenase, a gelatinase, a stromelysin, a matrilysin, a metalloelastase, or a membrane-type matrix metalloproteinase.

12. The method of claim 10, wherein the protease is matrix metalloproteinase-1, matrix metalloproteinase-2, matrix metalloproteinase-3, matrix metalloproteinase-4, matrix metalloproteinase-4, matrix metalloproteinase-5, matrix metalloproteinase-6, matrix metalloproteinase-7, matrix metalloproteinase-8, and matrix metalloproteinase-9, matrix metalloproteinase-10, matrix metalloproteinase-11, matrix metalloproteinase-12, matrix metalloproteinase-13, matrix metalloproteinase-14 or a combination thereof.

13. The method of claim 10, wherein the protease is matrix metalloproteinase-9.

14. The method of claim 10, wherein the activator is interleukin-1, thombopoietin, G-CSF, GMCSF, SDF-1, or fibroblast growth factor-4.

15. A method for increasing white blood cells in an animal's bloodstream comprising administering to the animal a protease or an activator of a protease.

16. The method of claim 15, wherein the protease mobilizes an endogenous population of quiescent non-cycling stem cells to a permissive vascular zone in the animal so that the stem cells proliferate, self-renew, or differentiate into white blood cells.

17. The method of claim 15, wherein the animal has been subjected to myelosuppressive stress.

18. The method of claim 15, wherein the protease is a matrix metalloproteinase, a collagenase, a gelatinase, a stromelysin, a matrilysin, a metalloelastase, or a membrane-type matrix metalloproteinase.

19. The method of claim 15, wherein the protease is matrix metalloproteinase-1, matrix metalloproteinase-2, matrix metalloproteinase-3, matrix metalloproteinase-4, matrix metalloproteinase-4, matrix metalloproteinase-5, matrix metalloproteinase-6, matrix metalloproteinase-7, matrix metalloproteinase-8, and matrix metalloproteinase-9, matrix metalloproteinase-10, matrix metalloproteinase-11, matrix metalloproteinase-12, matrix metalloproteinase-13, matrix metalloproteinase-14 or a combination thereof.

20. The method of claim 15, wherein the protease is matrix metalloproteinase-9.

21. The method of claim 15, wherein the activator is interleukin-1, thombopoietin, G-CSF, GMCSF, SDF-1, or fibroblast growth factor-4.

22. A method for stimulating hematopoiesis, angiogenesis or vasculogenesis in an animal comprising administering to the animal a protease or an activator of a protease.

23. The method of claim 22, wherein the protease mobilizes an endogenous population of quiescent non-cycling stem cells to a permissive vascular zone in the animal that supports proliferation or differentiation of the stem cells into hematopoietic or endothelial cells.

24. The method of claim 22, wherein the animal has been subjected to myelosuppressive stress.

25. The method of claim 22, wherein the quiescent non-cycling stem cells are in contact with bone marrow stromal cells, including osteoblasts.

26. The method of claim 22, wherein the quiescent non-cycling stem cells are maintained in a G₀ phase of cell cycle.

27. The method of claim 22, wherein the quiescent non-cycling stem cells are Lin⁻Sca⁺c-Kit⁺ hematopoietics stem cells, VEGFR2⁺c-Kit⁺ endothelial stem cells, AC133⁺VEGFR2⁺ vascular stem cells or AC133+ organ specific stem cells.

28. The method of claim 22, wherein the protease is a matrix metalloproteinase, a collagenase, a gelatinase, a stromelysin, a matrilysin, a metalloelastase, or a membrane-type matrix metalloproteinase.

29. The method of claim 22, wherein the protease is matrix metalloproteinase-1, matrix metalloproteinase-2, matrix metalloproteinase-3, matrix metalloproteinase-4, matrix metalloproteinase-4, matrix metalloproteinase-5, matrix metalloproteinase-6, matrix metalloproteinase-7, matrix metalloproteinase-8, and matrix metalloproteinase-9, matrix metalloproteinase-10, matrix metalloproteinase-11, matrix metalloproteinase-12, matrix metalloproteinase-13, matrix metalloproteinase-14 or a combination thereof.

30. The method of claim 22, wherein the protease is matrix metalloproteinase-9.

31. The method of claim 22, wherein the activator is interleukin-1, thombopoietin, G-CSF, GMCSF, SDF-1, or fibroblast growth factor-4.

32. A method for recruitment of adult stem cells in an animal comprising administering to the animal an effective amount of transgenic cells that overexpress a protease, wherein the recruitment of the stem cells translocates an endogenous population of quiescent non-cycling stem cells to a permissive vascular zone in the animal so that the stem cells proliferate, self-renew, differentiate or mobilize to a target site.

33. The method of claim 32, wherein the transgenic cells comprise a nucleic acid segment encoding the protease and the nucleic acid segment is operably linked to a second nucleic acid segment comprising a promoter that can initiate transcription of the protease in the transgenic cells.

34. The method of claim 32, wherein the target site is an injured tissue, a diseased tissue, a regenerating organ, a developing organ or bone.

35. The method of claim 32, wherein the target site is blood, bone marrow, cardiac tissue, hepatic tissue, lung tissue, kidney tissue, muscle tissue, neuronal tissue, vascular tissue or a combination thereof.

36. The method of claim 32, wherein the stem cells are hematopoietics stem cells, endothelial stem cells, hepatic stem cells, neuronal stem cells, muscle stem cells or a combination thereof.

37. The method of claim 32, wherein the protease is a matrix metalloproteinase, a collagenase, a gelatinase, a stromelysin, a matrilysin, a metalloelastase, or a membrane-type matrix metalloproteinase.

38. The method of claim 32, wherein the protease is matrix metalloproteinase-1, matrix metalloproteinase-2, matrix metalloproteinase-3, matrix metalloproteinase-4, matrix metalloproteinase-4, matrix metalloproteinase-5, matrix metalloproteinase-6, matrix metalloproteinase-7, matrix metalloproteinase-8, and matrix metalloproteinase-9, matrix metalloproteinase-10, matrix metalloproteinase-11, matrix metalloproteinase-12, matrix metalloproteinase-13, matrix metalloproteinase-14 or a combination thereof.

39. The method of claim 32, wherein the protease is matrix metalloproteinase-9.

40. The method of claim 32, wherein the activator is interleukin-1, thombopoietin, G-CSF, GMCSF, SDF-1, or fibroblast growth factor-4.

41. A method for recruitment of adult stem cells from a quiescent niche in an animal comprising administering to the quiescent niche a protease or an activator of a protease, wherein the recruitment translocates an endogenous population of quiescent non-cycling stem cells from the quiescent niche to a permissive vascular zone in the animal so that the stem cells can proliferate, self-renew, differentiate or mobilize to a target site.

42. The method of claim 41 wherein the quiescent niche is an osteoblastic niche.

43. The method of claim 1, wherein the animal is a mammal or a human.

44. A therapeutic method for treating a myeloproliferative disorder in a mammal comprising administering to the mammal an effective amount of an inhibitor of a matrix metalloproteinase.

45. The method of claim 44, wherein the myeloproliferative disorder is multiple myeloma.

46. A therapeutic method for treating a bone marrow failure disorder in a mammal comprising administering to the mammal an effective amount of a matrix metalloproteinase or an activator of a matrix metalloproteinase.

47. The method of claim 46, wherein the bone marrow failure disease is aplastic anemia.
